(19) United States
(12) Patent Application Publication
(10) Pub. No.: US 2013/0018010 A1
(43) Pub. Date: Jan. 17, 2013

(54) POLYMERIC CONJUGATES OF ADENINE NUCLEOSIDE ANALOGS

(71) Applicants: Hong Zhao, Edison, NJ (US); Jing Xia, Warren, NJ (US)

(72) Inventors: Hong Zhao, Edison, NJ (US); Jing Xia, Warren, NJ (US)

(73) Assignee: ENZON PHARMACEUTICALS, INC., Piscataway, NJ (US)

(21) Appl. No.: 13/637,297

(22) PCT Filed: Apr. 15, 2011
(86) PCT No.: PCT/US2011/032633

§ 371 Date: Oct. 9, 2012

Related U.S. Application Data
(60) Provisional application No. 61/325,059, filed on Apr. 16, 2010.
(60) Provisional application No. 61/325,050, filed on Apr. 16, 2010.

(51) Int. Cl. A61K 31/7064; A61P 31/12; C07H 19/16; A61P 35/00; A61K 31/7076; C07H 19/23

(52) U.S. Cl. 514/46; 514/49; 536/25.3; 536/27.2; 536/27.21

(57) ABSTRACT

The present invention relates to polymeric conjugates of adenine nucleoside analogs. In particular, the invention relates to multi-arm polyethylene glycol conjugates of adenine nucleoside analogs and use thereof. The present invention, more specifically, provides polymeric conjugates of toyocamycin and its derivatives. Furthermore, the present invention provides a method for preparing the polymeric conjugates of adenine nucleoside analogs and a method of using the same for treating a cancer, inhibiting the growth or proliferation of cancer cells, treating a viral infection, treating a disease or condition associated with abnormal expression of VEGF. Most polymeric conjugates of toyocamycin was stable in PBS but released toyocamycin in vivo to provided inhibition of cancer cell growth.

### CROSS-REFERENCE TO RELATED APPLICATIONS

This application claims the benefit of priority from U.S. Provisional Patent Application Ser. Nos. 61/325,050 and 61/325,059 filed Apr. 16, 2010, the contents of each of which are incorporated herein by reference.

### FIELD OF THE INVENTION

The present invention relates to polymeric conjugates of adenine nucleoside analogs. In particular, the invention relates to multi-arm polyethylene glycol conjugates of adenine nucleoside analogs and use thereof.

### BACKGROUND OF THE INVENTION

A number of nucleoside analogs, which are structurally similar to natural nucleosides, have shown useful therapeutic activities. Many of adenine nucleoside analogs are reported to be cytotoxic and induce apoptosis. Such nucleoside analogs have been shown to be potent anticancer agents. For example, toyocamycin and related analogs are known to be potential anticancer agents and have demonstrated therapeutic activity in vitro and in vivo. Toyocamycin is also known to inhibit RNA processing, RNA self-cleavage and VEGF secretion. It is reported that toyocamycin-based therapy showed adverse GI side effects in preclinical studies. Clinical trials involving with toyocamycin therapy have been discontinued due to severe toxicities and side effects, such as local necrosis.

Over the years, several methods of administering biologically-effective materials to mammals have been proposed. Many medicinal agents are available as water-soluble salts and can be included in pharmaceutical formulations relatively easily. Problems arise when the desired medicinal agent is either insoluble in aqueous fluids or is rapidly degraded in vivo or eliminated too quickly before it can provide sufficient therapeutic activity. Adenine nucleoside analogs often encounter water solubility problems and also short residency time in vivo. Thus, it would be advantageous to provide artisans with alternative and/or improved technology for delivery of biologically active adenine nucleoside analogs.

### SUMMARY OF THE INVENTION

In order to improve the technology for adenine nucleoside analog-based therapy, the present invention provides delivery systems for adenine nucleoside analogs. In one aspect of the present invention, there are provided compounds of Formula (I) or (Ia):

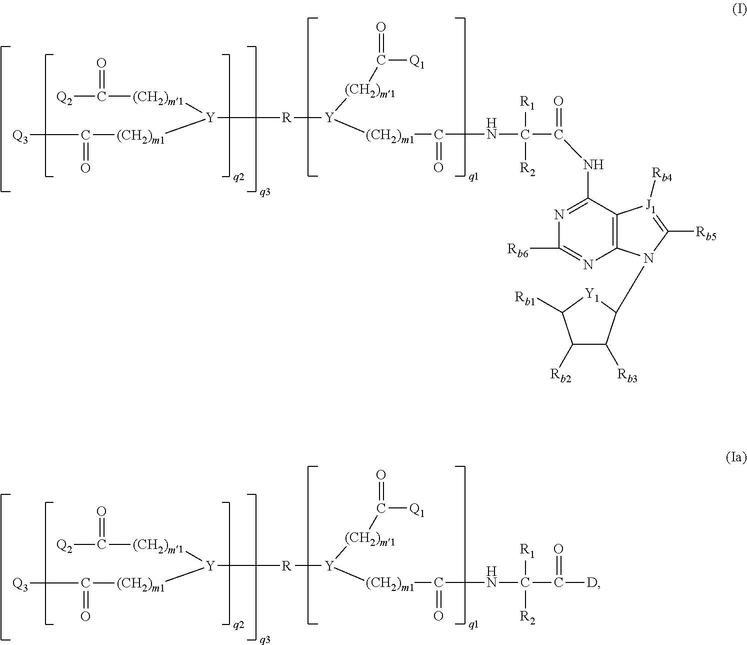

wherein

R is a substantially non-antigenic polymer having one to about 32 polymer arms;

Y is —NHCH— or N;

Q₁, Q₂, and Q₃, in each occurrence, are independently OH, a leaving group,

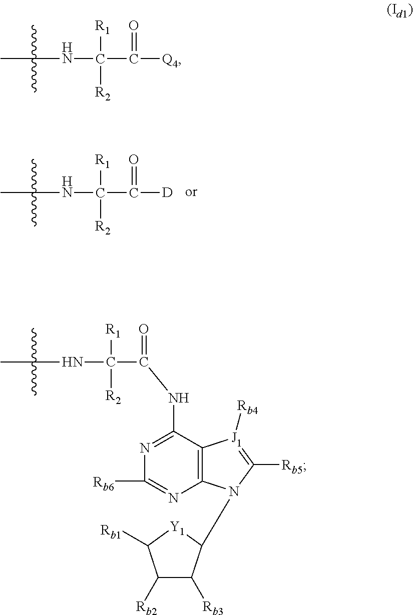

Q₄, in each occurrence, is independently OH or a leaving group;

R₁, in each occurrence, is independently H, C₁₋₁₀ alkyl, C₃₋₁₀ branched alkyl, C₃₋₈ cycloalkyl, C₂₋₁₀ alkenyl or C₂₋₁₀ alkynyl;

R₂, in each occurrence, is independently C₁₋₁₀ alkyl, C₃₋₁₀ branched alkyl, C₃₋₈ cycloalkyl, C₂₋₁₀ alkenyl or C₂₋₁₀ alkynyl;

J₁, in each occurrence, is independently C or N;

Y₁, in each occurrence, is independently O, S, or CH₂;

D is:

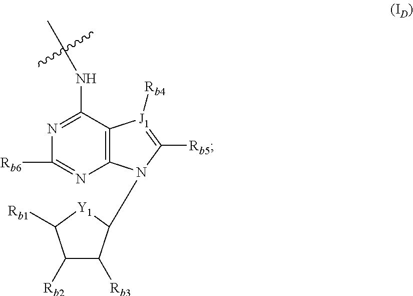

R_{b1}, in each occurrence, is independently hydrogen, hydroxyl, C₂₋₁₀ alkenyl, C₂₋₁₀ alkynyl, —(CH₂)ₘ₂—OR_{c1} or —(CH₂)ₘ₂—R′_{c1};

R_{b2}, in each occurrence, is independently hydrogen, hydroxyl, C₂₋₁₀ alkenyl, C₂₋₁₀ alkynyl, C₂₋₁₀ alkenyloxy, C₃₋₁₀ alkyloxy, halogen, azido, amino, or OR_{c2};

R_{b3}, in each occurrence, is independently hydrogen, hydroxyl, C₂₋₁₀ alkenyl, C₂₋₁₀ alkynyl, C₂₋₁₀ alkenyloxy, C₃₋₁₀ alkyloxy, halogen (F, Cl, or Br), azido, amino, or OR_{c3};

R_{b4}, in each occurrence, is independently hydrogen, halogen, C₁₋₁₀ alkyl, aryl, aralkyl, C₂₋₁₀ alkenyl, C₂₋₁₀ alkynyl, C₁₋₁₀ alkoxy, cyano, cyanoalkyl, —C(═O)NH₂, carboxyamido, aryloxy, amino, alkylamino, arylamino, aralkylamino, alkylthio, or arylthio, when J₁ is carbon, and is null when J₁ is nitrogen;

R_{b5}, in each occurrence, is independently hydrogen, amine, halogen, C₁₋₁₀ alkyl, alkylamino, alkylthio, —NH—NH₂, or azido;

R_{b6}, in each occurrence, is independently hydrogen, C₁₋₁₀ alkyl (lower alkyl), halogen, C₁₋₁₀ alkoxy, or C₁₋₁₀ alkylthio;

R_{c1}, in each occurrence, is independently hydrogen, C₁₋₁₀ acyl, monophosphate, diphosphate, triphosphate, C₁₋₁₀ alkyl, C₃₋₈ cycloalkyl, C₂₋₁₀ alkenyl, C₂₋₁₀ alkynyl, or a substantially non-antigenci polymer;

R′_{c1}, in each occurrence, is independently hydrogen, hydroxyl, lower alkyl esters or carbonate esters thereof, C₁₋₁₀ alkyl, C₁₋₁₀ alkoxy, amino, azido, halogen or a substantially non-antigenci polymer;

R_{c2}, in each occurrence, is independently hydrogen, C₁₋₁₀ acyl, monophosphate, diphosphate, triphosphate C₁₋₁₀ alkyl, C₃₋₈ cycloalkyl, C₂₋₁₀ alkenyl, C₂₋₁₀ alkynyl, or a substantially non-antigenic polymer;

R_{c3}, in each occurrence, is independently hydrogen, C₁₋₁₀ acyl, monophosphate, diphosphate, triphosphate, —CH₂CH₂OH, or CH₂CH₂F, C₁₋₁₀ alkyl, C₃₋₈ cycloalkyl, C₂₋₁₀ alkenyl, C₂₋₁₀ alkynyl, or a substantially non-antigenic polymer;

(m1) and (m′1) are independently zero, 1, or 2, provided that (m1) and (m′1) are independently 1 or 2, when Y is N;

(m2) is an integer from about 1 to about 4;

(q1) and (q2) are independently zero or 1; and

(q3) is zero or a positive integer of from about 1 to about 31.

In one preferred aspect, an adenine nucleoside analog is attached via the amine thereof to each polymer arm terminal of a multi-arm polymer through a spacer. Less than complete loading may occur. Alternatively, at least about 50% (preferably at least about 75%) of the arms include an adenine nucleoside analog.

Methods of making and using the compounds as well as methods of treatment using the compounds of the present invention are also provided.

The present invention provides drug delivery systems for adenine nucleoside analogs such as toyocamycin, which allow them to retain substantially all of their inherent pharmacological advantages, and while at the same time reducing some of the severe toxicities and adverse side effects associated with adenine nucleoside analog-based therapy (e.g., adverse GI side effects in toyocamycin-based therapy).

Additional advantages of the present invention will be apparent from the following description and drawings.

For purposes of the present invention, the term “residue” shall be understood to mean that portion of a compound, to which it refers, i.e. an adenine nucleoside analog (e.g., toyocamycin), a spacer, a branching group, polyethylene glycol, etc. that remains after it has undergone a substitution reaction with another compound.

For purposes of the present invention, the term “polymeric residue” or “PEG residue” shall each be understood to mean that portion of the polymer or PEG which remains after it has undergone a reaction with, e.g., a spacer, a branching group.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably includes halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkoxy group.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “halogen’ or “halo” refers to fluorine, chlorine, bromine, and iodine.

For purposes of the present invention, the term “heteroatom” refers to nitrogen, oxygen, and sulfur.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, “positive integer” shall be understood to include an integer equal to or greater than 1 (e.g., 1, 2, 3, 4, 5, 6) and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art. An effective amount for each mammal or human patient to be treated is readily determined by the artisan in a range that provides a desired clinical response while avoiding undesirable effects that are inconsistent with good practice. Dose ranges are provided hereinbelow.

For purposes of the present invention, the terms “cancer” and “tumor” are used interchangeably, unless otherwise indicated. Cancer encompasses benign, malignant and/or metastatic cancer, unless otherwise indicated. Cancers may be more aggressive or less aggressive. The aggressive phenotype refers to the proliferation rate and the ability to form tumors and metastasize. Aggressive cancers proliferate more quickly, and form tumors and metastasize more easily, as compared to less-aggressive tumors.

For purposes of the present invention, “treatment of tumor/cancer” shall be understood to mean inhibition, reduction, and amelioration of tumor growth, tumor burden and metastasis, remission of tumor, or reduction of recurrences of tumor and/or neoplastic growths realized in patients after completion of the therapy with the compound described herein, as compared to patients who have not received the treatment described herein. Successful treatment is deemed to occur when a patient achieves positive clinical results. For example, successful treatment of a tumor shall be deemed to occur when at least 10% or preferably 20%, more preferably 30% or higher (i.e., 40%, 50%) decrease in tumor growth including other clinical markers contemplated by the artisan in the field is realized when compared to that observed in the absence of the treatment described herein. Other methods for determining changes in a tumor clinical status resulting from the treatment described herein include: biopsies such as a tumor biopsy, an immunohistochemistry study using antibody, radioisotope, dye, and complete blood count (CBC).

For purposes of the present invention, use of phrases such as “decreased”, “reduced”, “diminished”, or “lowered” includes at least a 10% change in pharmacological activity with greater percentage changes being preferred (for reduction in tumor growth or, if relevant, gene/protein expression associated with tumor). For instance, the change may also be greater than 25%, 35%, 45%, 55%, 65%, or other increments greater than 10%, or the range may be in a range from 25% through 99%.

The term “at least about” comprises the numbers equal to or larger to the numbers. In various embodiments, such as when referring to the decrease in tumor growth and gene/protein expression associated with tumor, the term “at least about 15%” includes the terms “at least about 16%”, “at least about 17%”, at least about 18%” and so forth. Likewise, in some embodiments, the term “at least about 30%” includes the terms “at least about 31%”, “at least about 32%”, and so forth.

### BRIEF DESCRIPTION OF THE DRAWINGS

FIG. 1 schematically illustrates a reaction scheme of preparing compound 6 as described in Examples 5-7.

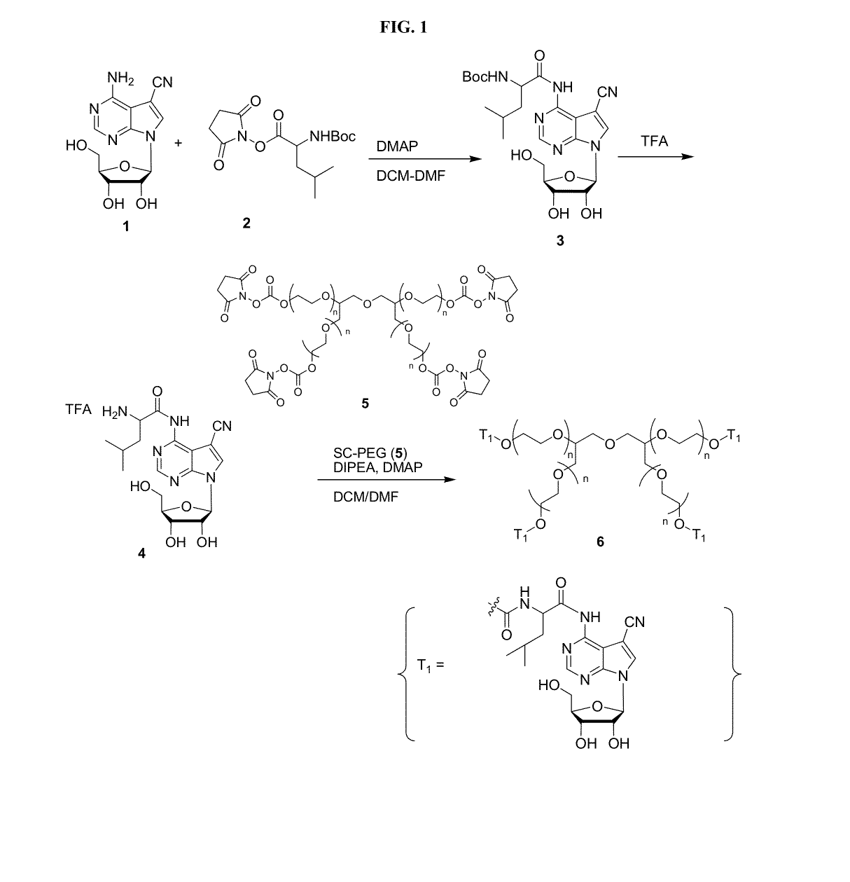

FIG. 2 schematically illustrates a reaction scheme of preparing compound 10 as described in Examples 8-10.

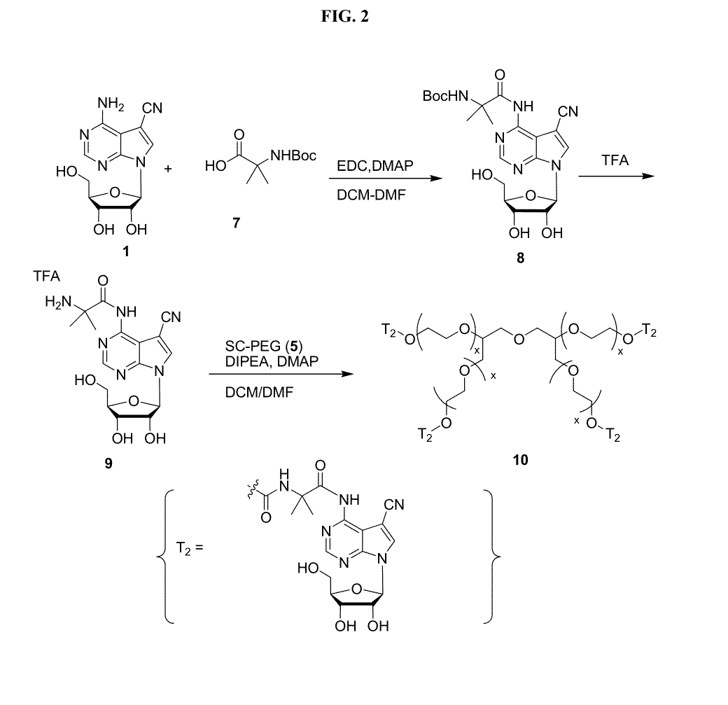

FIG. 3 schematically illustrates a reaction scheme of preparing compound 14 as described in Examples 11-13.

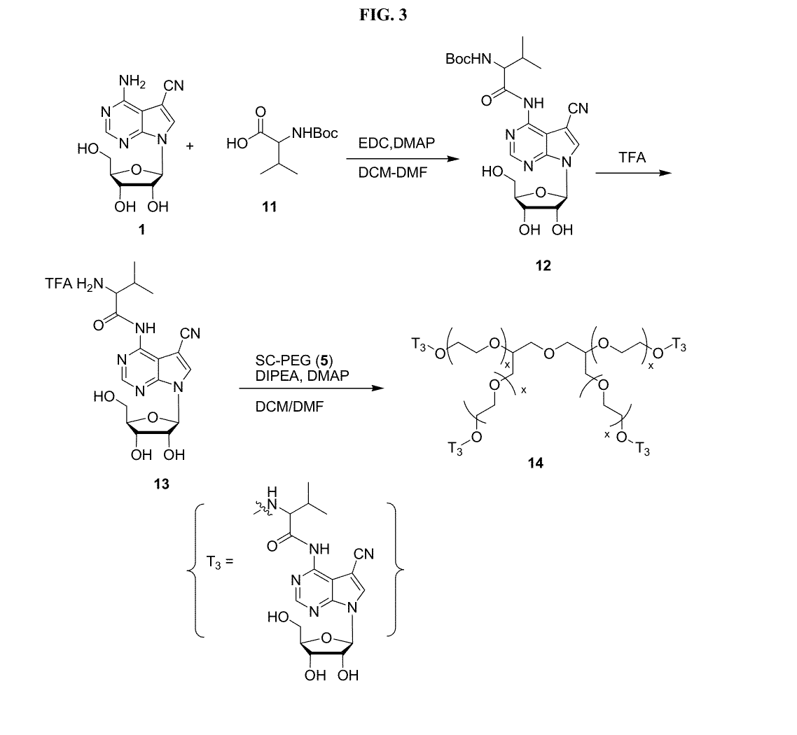

FIG. 4 schematically illustrates a reaction scheme of preparing compound 19 as described in Examples 14-16.

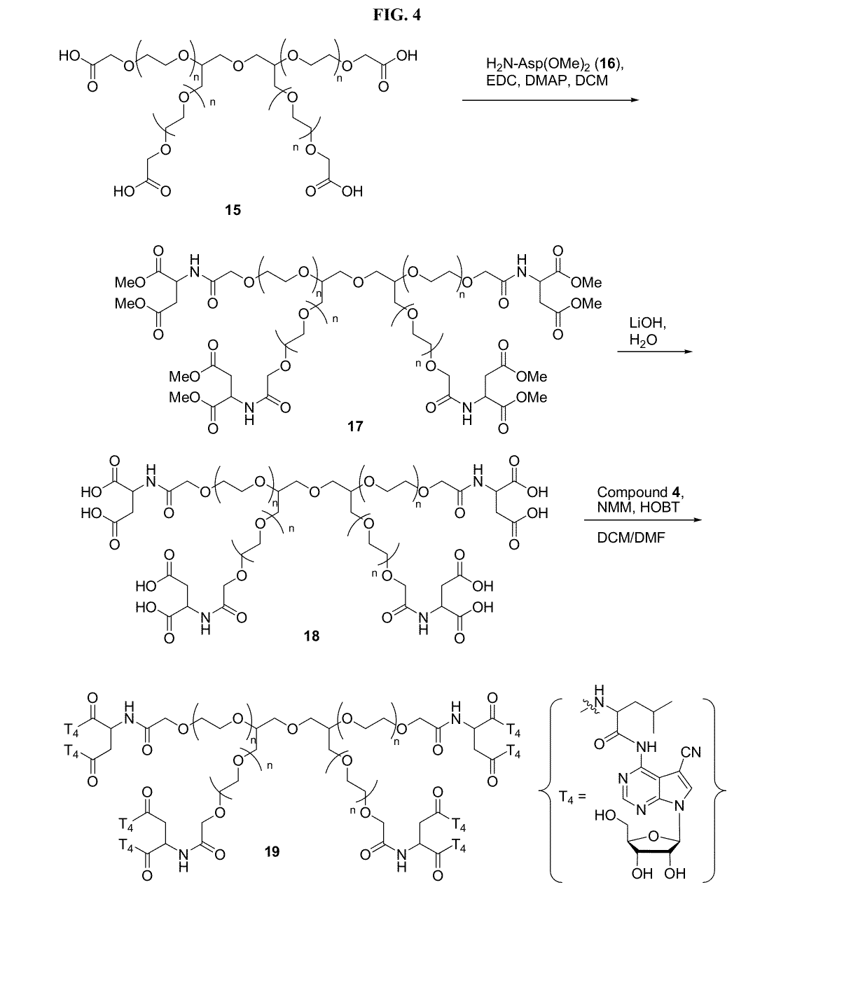

FIG. 5 schematically illustrates a reaction scheme of preparing compound 23 as described in Examples 17-19.

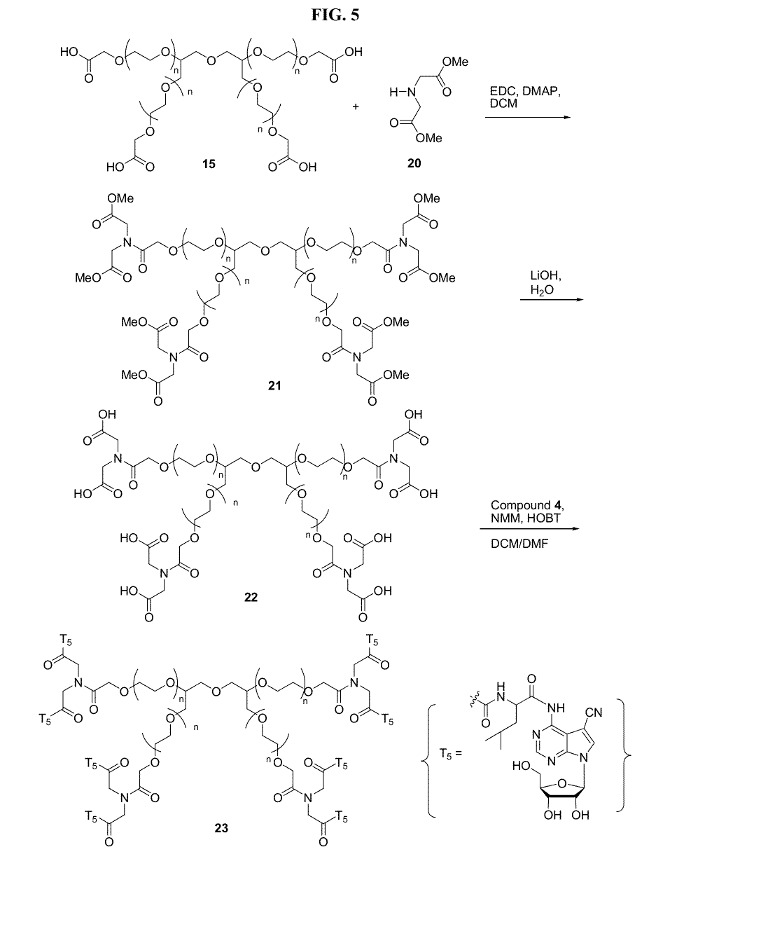

FIG. 6 schematically illustrates a reaction scheme of preparing compounds 25 and 26 as described in Examples 20-21.

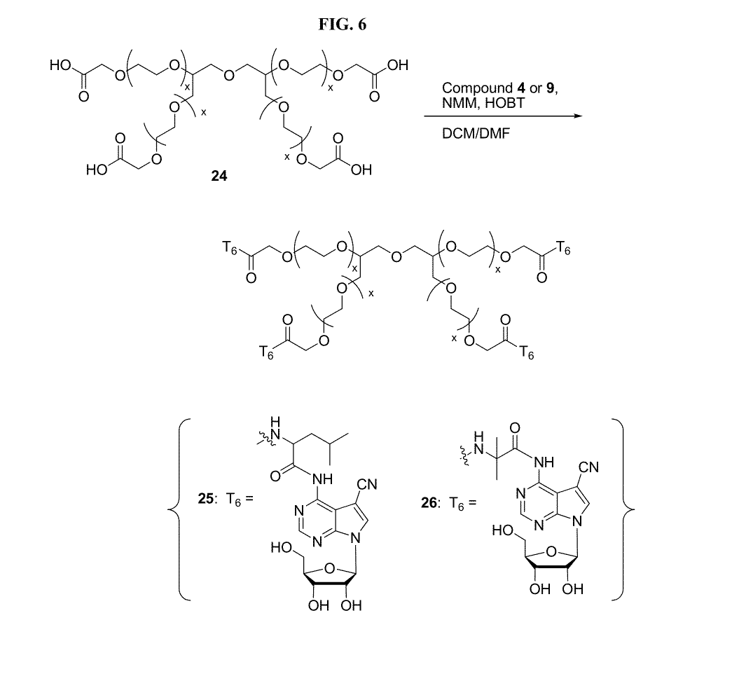

FIG. 7 schematically illustrates a reaction scheme of preparing compounds 28 and 29 as described in Examples 22-23.

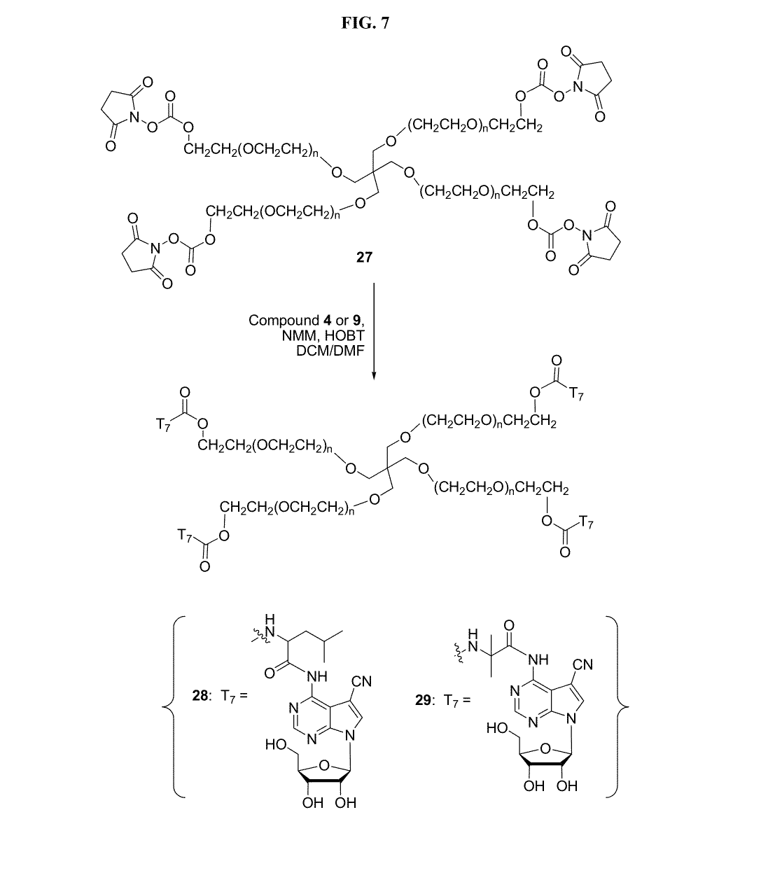

FIG. 8 schematically illustrates a reaction scheme of preparing compound 33 as described in Examples 24-26.

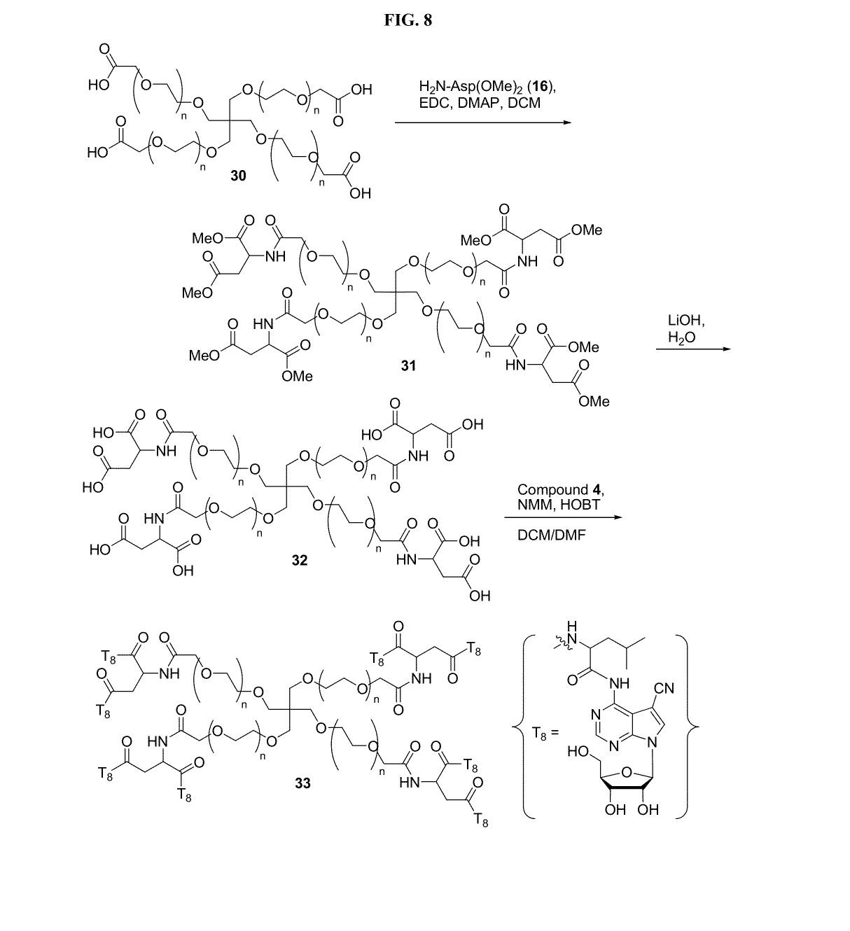

FIG. 9 schematically illustrates a reaction scheme of preparing compound 36 as described in Examples 27-29.

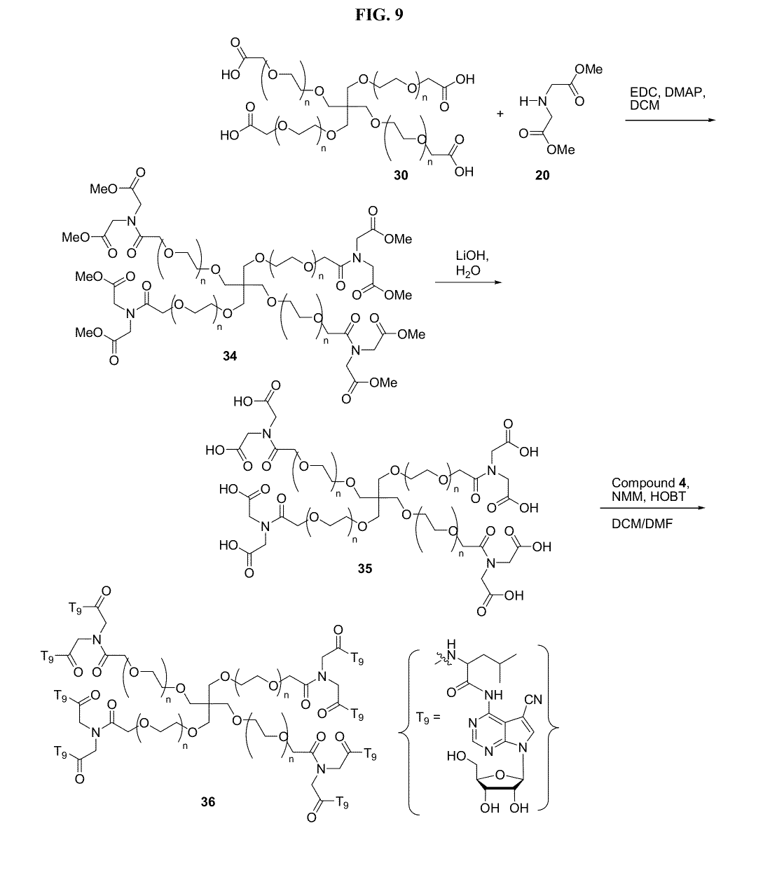

FIG. 10 schematically illustrates a reaction scheme of preparing compounds 38 and 39 as described in Examples 30-31.

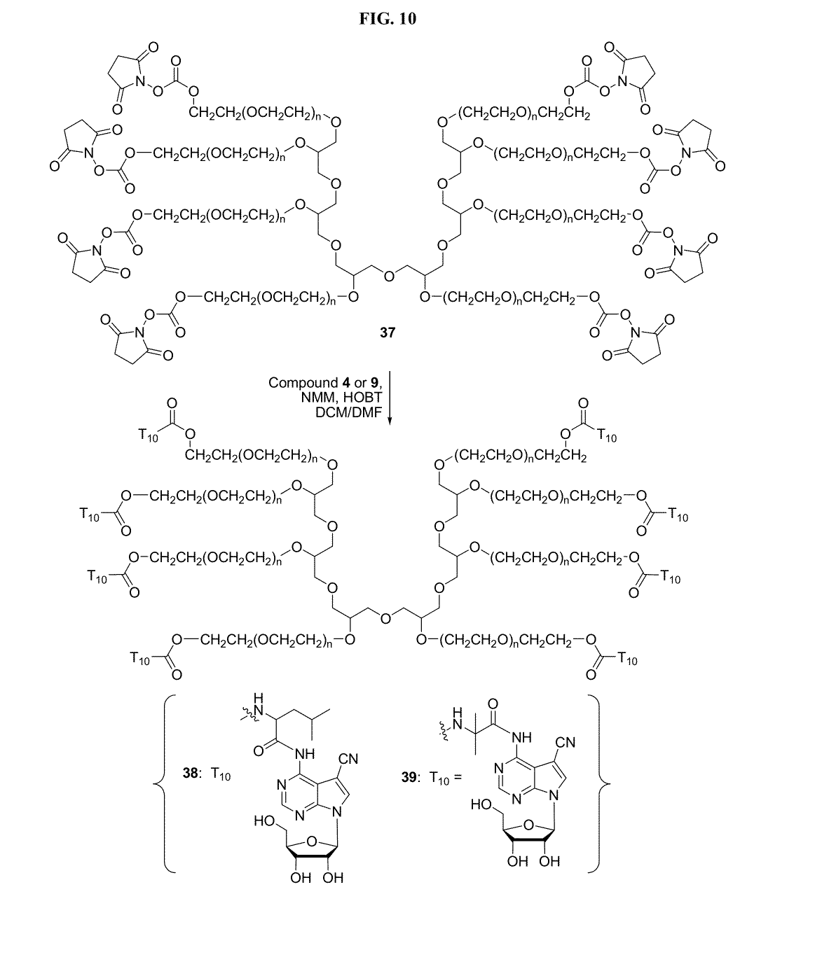

FIG. 11 schematically illustrates a reaction scheme of preparing compound 42 as described in Examples 32-33.

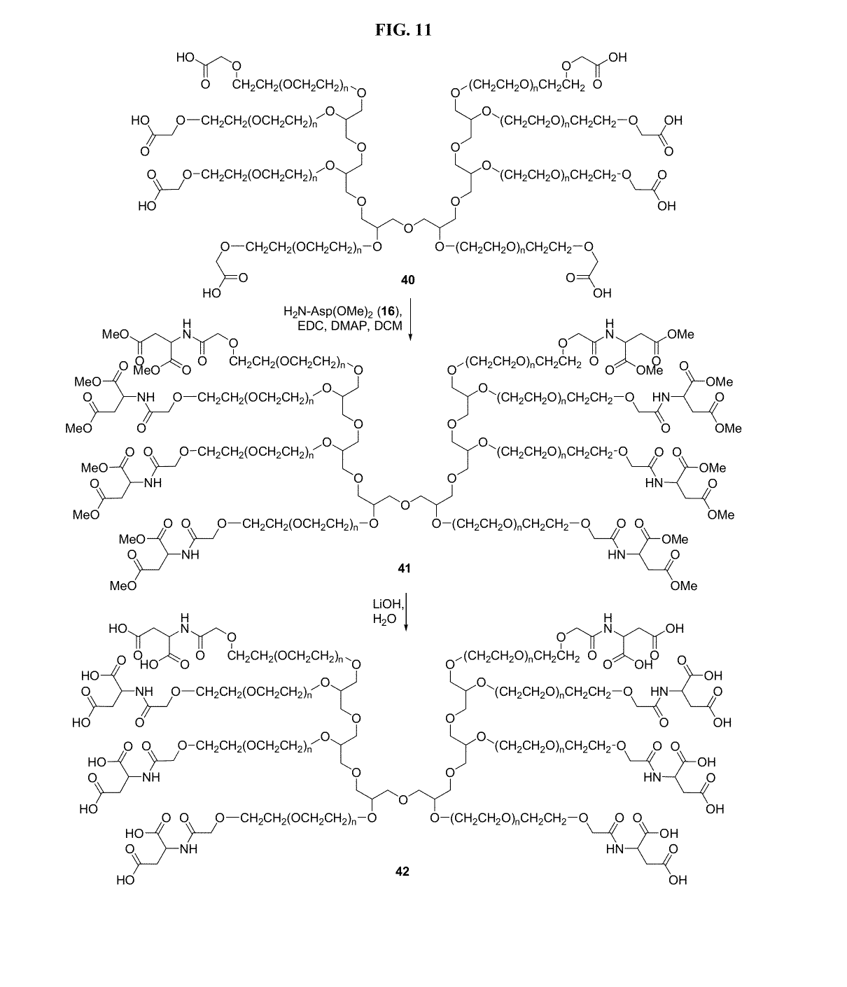

FIG. 12 schematically illustrates a reaction scheme of preparing compound 43 as described in Examples 34.

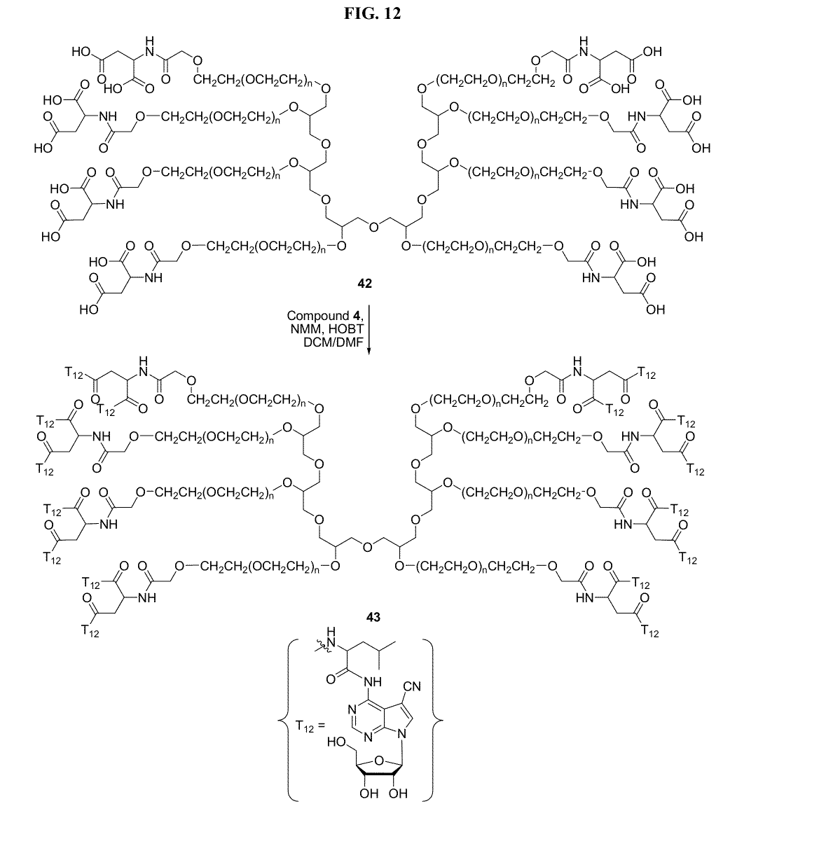

FIG. 13 schematically illustrates a reaction scheme of preparing compound 44 as described in Examples 35.

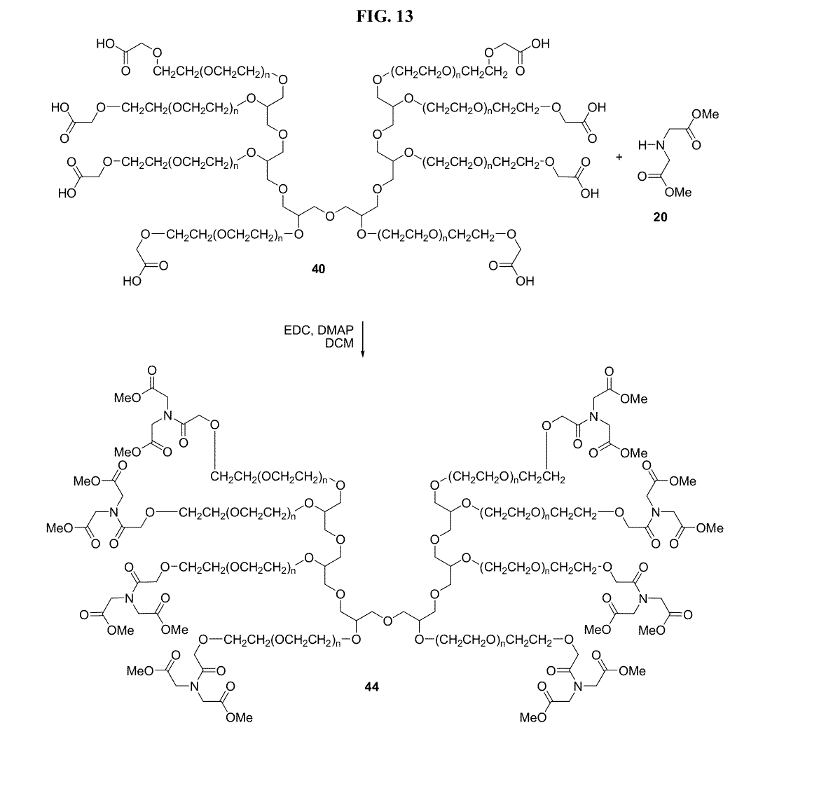

FIG. 14 schematically illustrates a reaction scheme of preparing compound 45 as described in Examples 36.

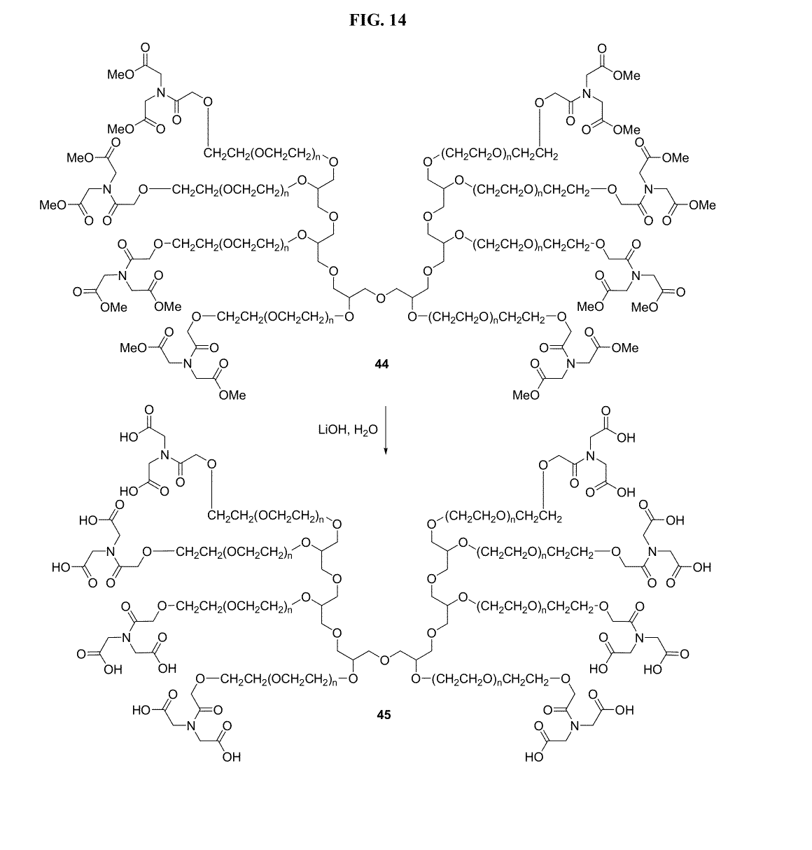

FIG. 15 schematically illustrates a reaction scheme of preparing compound 46 as described in Examples 37.

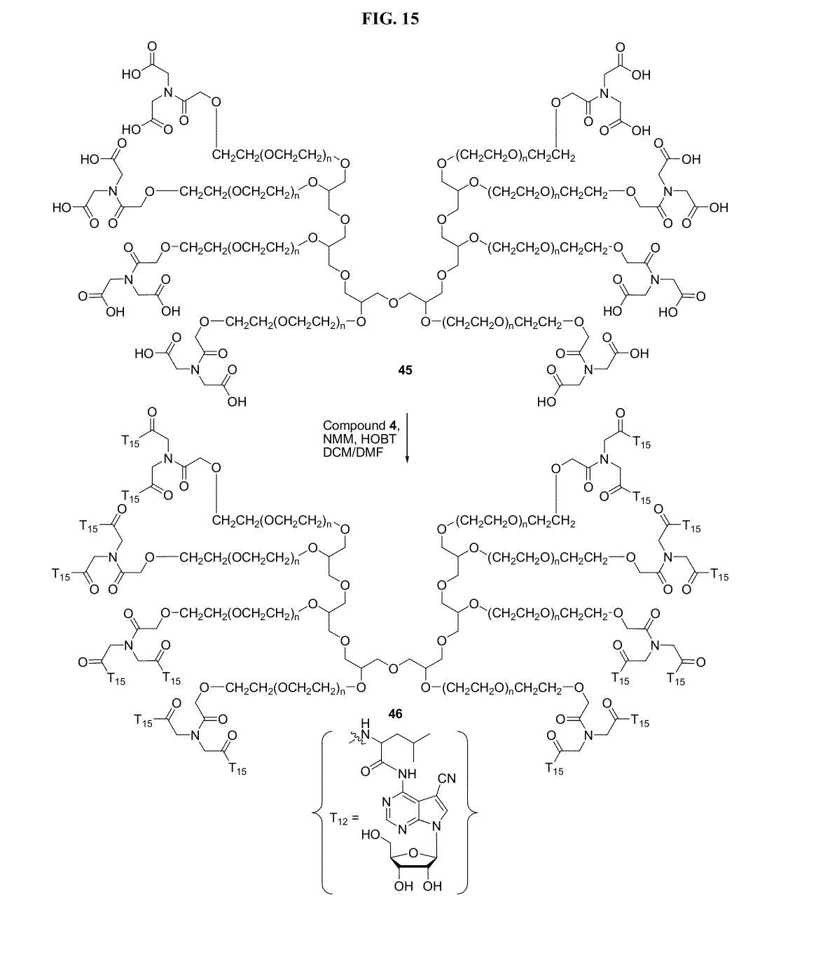

FIG. 16 schematically illustrates a reaction scheme of preparing compounds 48 and 49 as described in Examples 38-39.

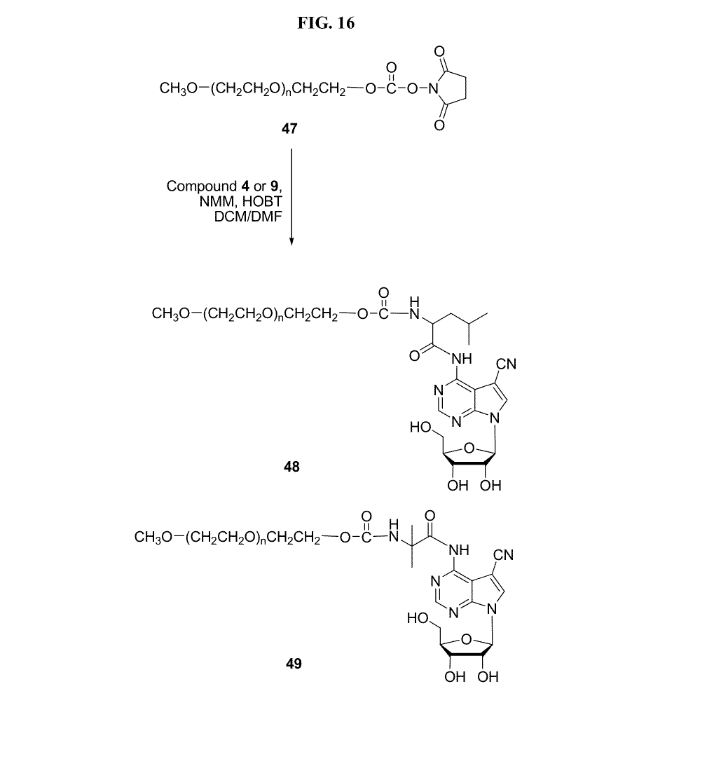

FIG. 17 schematically illustrates a reaction scheme of preparing compounds 54 as described in Examples 40-42.

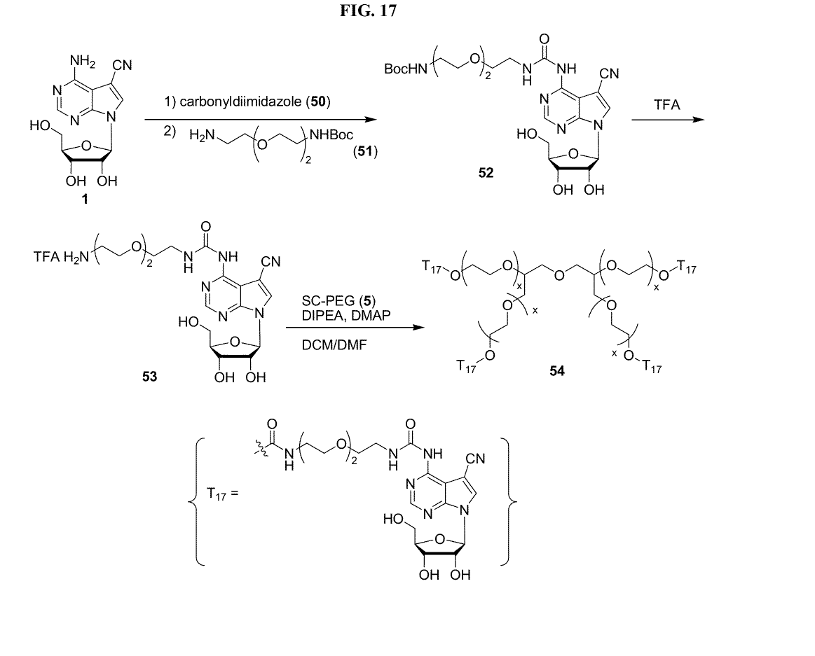

FIG. 18 illustrates antitumor efficacy of toyocamycin, compound 6, compound 10 and compound 54 in mice xenografted with human melanoma cells, as described in Example 44.

### DETAILED DESCRIPTION OF THE INVENTION

### A. Overview

In one aspect of the present invention, there are provided compounds of Formula (Ia) or (I):

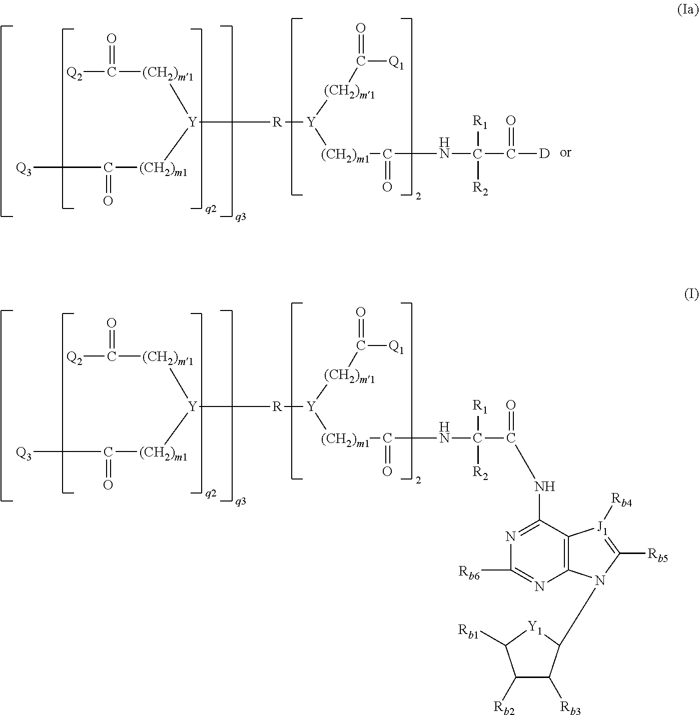

wherein

R is a substantially non-antigenic polymer having one to about 32 polymer arms;

Y is —NHCH— or N, which, as included in Formula (I), corresponds to

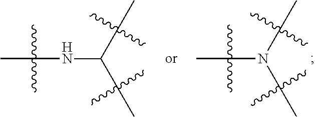

Q₁, Q₂, and Q₃, in each occurrence, is independently OH, a leaving group,

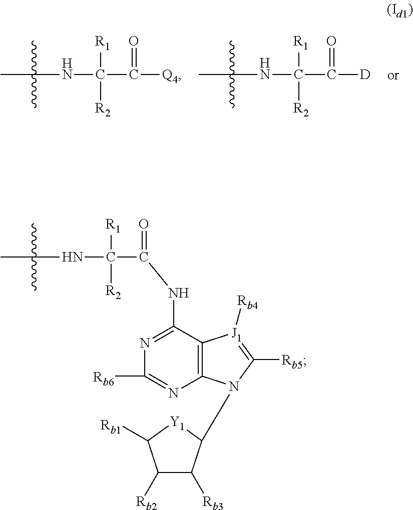

Q₄, in each occurrence, is independently OH or a leaving group;

R₁, in each occurrence, is independently H, C₁₋₁₀ alkyl, C₃₋₁₀ branched alkyl, C₃₋₈ cycloalkyl, C₂₋₁₀ alkenyl or C₂₋₁₀ alkynyl;

R₂, in each occurrence, is independently C₁₋₁₀ alkyl, C₃₋₁₀ branched alkyl, C₃₋₈ cycloalkyl, C₂₋₁₀ alkenyl or C₂₋₁₀ alkynyl;

J₁, in each occurrence, is independently C or N;

Y₁, in each occurrence, is independently O, S, or CH₂;

D is:

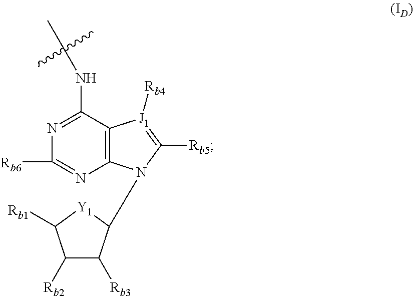

R_{b1}, in each occurrence, is independently hydrogen, hydroxyl, C₂₋₁₀ alkenyl, C₂₋₁₀ alkynyl, —(CH₂)ₘ₂—OR_{c1} or —(CH₂)ₘ₂—R′_{c1};

R_{b2}, in each occurrence, is independently hydrogen, hydroxyl, C₂₋₁₀ alkenyl, C₂₋₁₀ alkynyl, C₂₋₁₀ alkenyloxy, C₃₋₁₀ alkyloxy, halogen, azido, amino, or OR_{c2};

R_{b3}, in each occurrence, is independently hydrogen, hydroxyl, C₂₋₁₀ alkenyl, C₂₋₁₀ alkynyl, C₂₋₁₀ alkenyloxy, C₃₋₁₀ alkyloxy, halogen (F, Cl, or Br), azido, amino, or OR_{c3};

R_{b4}, in each occurrence, is independently hydrogen, halogen, C₁₋₁₀ alkyl, aryl, aralkyl, C₂₋₁₀ alkenyl, C₂₋₁₀ alkynyl, C₁₋₁₀ alkoxy, cyano, cyanoalkyl, —C(═O)NH₂, carboxyamido, aryloxy, amino, alkylamino, arylamino, aralkylamino, alkylthio, or arylthio, when J₁ is carbon, and is null when J₁ is nitrogen;

R_{b5}, in each occurrence, is independently hydrogen, amine, halogen, C₁₋₁₀ alkyl, alkylamino, alkylthio, —NH—NH₂, or azido;

R_{b6}, in each occurrence, is independently hydrogen, C₁₋₁₀ alkyl (lower alkyl), halogen (F, Cl), C₁₋₁₀ alkoxy, or C₁₋₁₀ alkylthio;

R_{c1}, in each occurrence, is independently hydrogen, C₁₋₁₀ acyl, monophosphate, diphosphate, triphosphate, C₁₋₁₀ alkyl, C₃₋₈ cycloalkyl, C₂₋₁₀ alkenyl, C₂₋₁₀ alkynyl, or a substantially non-antigenci polymer;

R′_{c1}, in each occurrence, is independently hydrogen, hydroxyl, lower alkyl esters or carbonate esters thereof, C₁₋₁₀ alkyl, C₁₋₁₀ alkoxy, amino, azido, halogen or a substantially non-antigenci polymer;

R_{c2}, in each occurrence, is independently hydrogen, C₁₋₁₀ acyl, monophosphate, diphosphate, triphosphate C₁₋₁₀ alkyl, C₃₋₈ cycloalkyl, C₂₋₁₀ alkenyl, C₂₋₁₀ alkynyl, or a substantially non-antigenic polymer;

R_{c3}, in each occurrence, is independently hydrogen, C₁₋₁₀ acyl, monophosphate, diphosphate, triphosphate, —CH₂CH₂OH, or CH₂CH₂F, C₁₋₁₀ alkyl, C₃₋₈ cycloalkyl, C₂₋₁₀ alkenyl, C₂₋₁₀ alkynyl, or a substantially non-antigenic polymer;

(m1) and (m′1) are independently zero, 1, or 2, provided that (m1) and (m′1) are independently 1 or 2, when Y is N;

(m2) is an integer from about 1 to about 4 (e.g., 1, 2, 3, 4);

(q1) and (q2) are independently zero or 1; and

(q3) is zero or a positive integer of from about 1 to about 31, preferably, 0, 1, 3, 7, 15, 31;

In this aspect, R further includes a capping group (A) such as H, OH, C₁₋₆ alkyl, C₁₋₆ alkoxy, COOH, or NH₂, when (q3) is zero.

According to the present invention, the compounds described herein are provided in which the number of the adenine nucleoside analogs contained in the compound of Formula (I) ranges from about 1 to about 64, (e.g, 1, 2, 3, 4, 5, 6, 7, 8, 9, 10, 11, 12, 13, 14, 15, 16, and the like).

For purposes of the present invention, the term “adenine nucleoside analog” shall be understood to include ribonucleosides, deoxyribnucleosides, ribonucleotides, deoxyribonucleotides, and their derivatives in which their nucleobases include adenine and 7-deazaadenine. In one aspect, adenine nucleoside analogs, as included in the compounds of the present invention, are denoted by such as D group having the Formula (I_{D}):

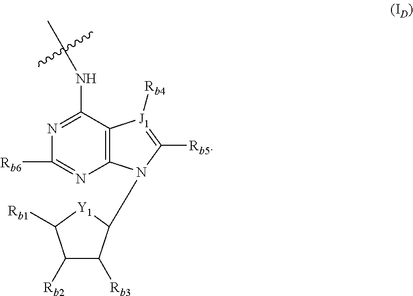

Examples of adenine nucleoside analogs include, but are not limited to, adenosine, 2′-deoxyadenosine, toyocamycin, sangivamycin (NSC 65346), ARC(NSC 188491), fludarabine, cladribine, clofarabine, and mono-, di- or tripphosphate thereof, etc. Adenine nucleoside analogs, as included in the compounds of Formula (I), include:

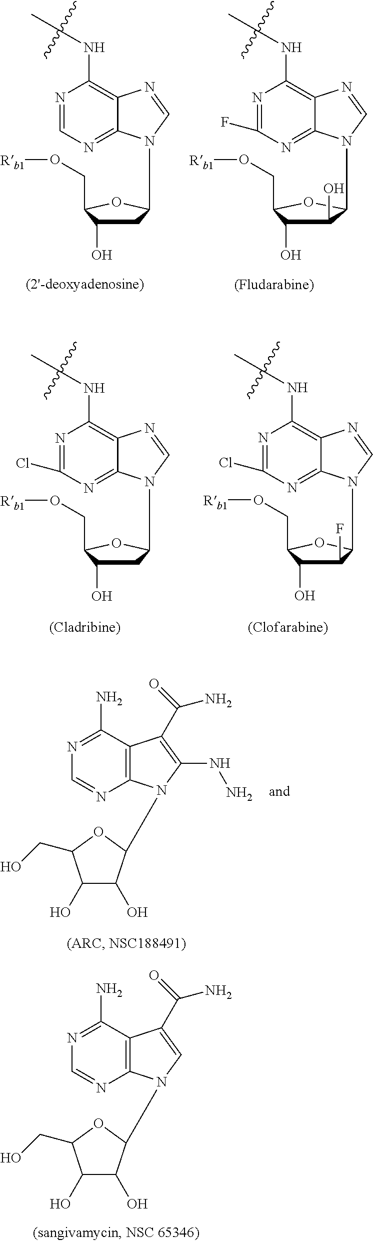

wherein R′_{b1} is hydrogen, mono-, di-, or triphosphate.

Additional adenine analogs contemplated according to the present invention are described in U.S. Pat. Nos. 5,506,347; 5,674,998; 5,721,356; 5,726,302; 5,763,596; 5,763,597; 5,750,673; 6,987,177; 6,670,468; and 7,608,600, the content of each of which are incorporated herein by reference.

Preferably, the adenine analogs are 7-deazaadenine ribonucleosides such as toyocamycin. The compounds of the present invention are provided in which the biologically active agents are toyocamycin, sangivamycin (NSC 65346), ARC(NSC 188491), 6-aminotoyocamycin, tubercidin, and mono-, di-, or triphosphate thereof. According to the present invention, toyocamycin and analogs thereof, as included in Formula (I), have the formula:

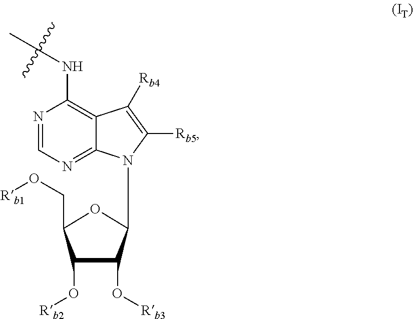

wherein

R′_{b1}, R′_{b2} and R′_{b3} are independently hydrogen, monophosphate, diphosphate, or triphosphate;

R_{b4} is —CN, —C(═O)NH₂, or hydrogen; and

R_{b5} is hydrogen, amine, or —NH—NH₂,

or a pharmaceutical salt thereof. In this respect, J₁ is carbon.

In one preferred embodiment, the compounds of Formula (I) include toyocamycin, wherein R′_{b1}, R′_{b2} and R′_{b3} are all hydrogen, R_{b4} is cyano, and R_{b5} is hydrogen.

In another preferred embodiment, the compounds of Formula (I) include sangivamycin, wherein R′_{b1}, R′_{b2} and R′_{b3} are all hydrogen, R_{b4} is —C(═O)NH₂, and R_{b5} is hydrogen.

In another embodiment, the compounds of Formula (I) include ARC, wherein R′_{b1}, R′_{b2} and R′_{b3} are all hydrogen, R_{b4} is —C(═O)]—NH₂, and R_{b5} is —NH—NH₂.

In another embodiment, the compounds of Formula (I) include tubercidin, wherein R′_{b1}, R′_{b2} and R′_{b3} are all hydrogen, R_{b4} is hydrogen, and R_{b5} is hydrogen.

In another embodiment, the compounds of Formula (I) include 6-aminotoyocamycin, wherein R′_{b1}, R′_{b2} and R′_{b3} are all hydrogen, R_{b4} is cyano, and R_{b5} is —NH₂.

In yet another embodiment, the compounds of Formula (I) include phosphates (mono-, di-, or triphosphate) where R_{b1} is —CH₂OR′_{b1}, and R′_{b1} is mono-, di-, or triphosphate.

In preferred embodiments, the compounds of Formula (I) include:

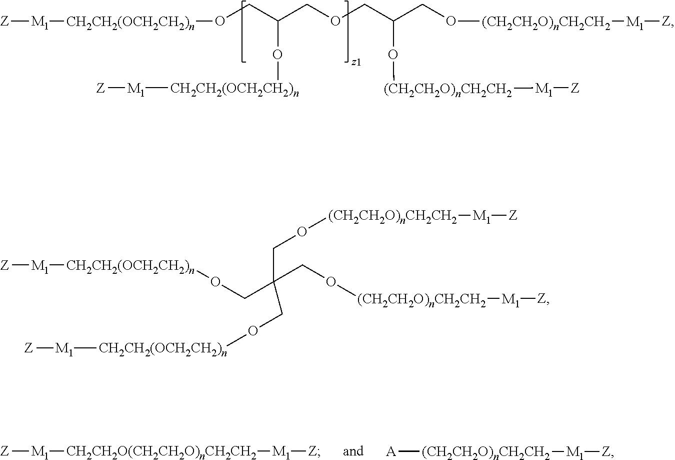

wherein

M₁ is independently O, or S;

Z, each occurrence, is independently H,

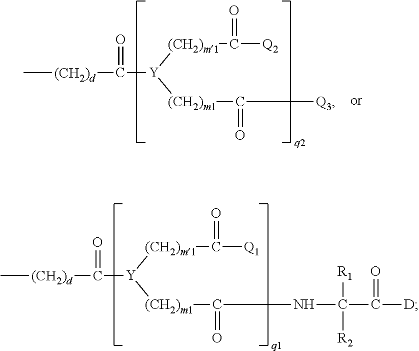

Y, in each occurrence, is —NHCH— or N;

Q₁, Q₂, and Q₃, in each occurrence, are independently OH, a leaving group,

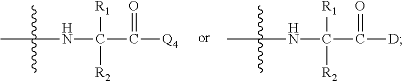

Q₄, in each occurrence, is independently OH or a leaving group;

D is

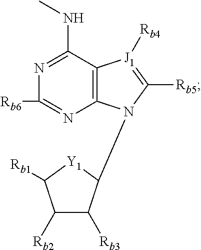

A is OH, C₁₋₆ alkoxy, COOH, or NH₂, preferably OH, methoxy, or ethoxy, (in this respect, (q3) is zero);

(d) is zero or a positive integer of from about 1 to about 10, preferably, 0-4, and more preferably, zero, for 2;

(z1) is zero or a positive integer of from 1 to about 29 (e.g., 0, 1, 2, 3, 4, 5, 6, 7, 8, 9, 10, 11, 12, 13, and the like, preferably, 1, 5, 13, 29);

(n) is a positive integer of from about 10 to about 2,300 so that the total number average molecular weight of the polymeric portion of the compound ranges from about 2,000 to about 100,000 daltons,

provided that one or more (e.g., 1, 2, 3, 4, 5, 6, 7, 8) of Z are

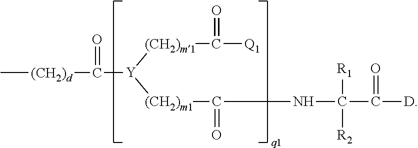

The compounds of Formula (I) include one or more adenine nucleoside analogs (such as toyocamycin) attached via a Z group. Z is the following:

(i) Z is H,

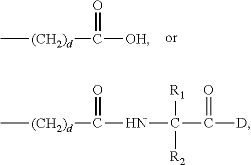

wherein
(d) is zero, 1 or 2; and
at least one, preferably more (e.g., 1, 2, 3, 4, 5, 6, 7, 8) of Z are

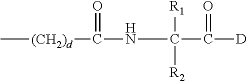

(in this respect, (q1) and (q2) are both zero); or

(ii) Z is

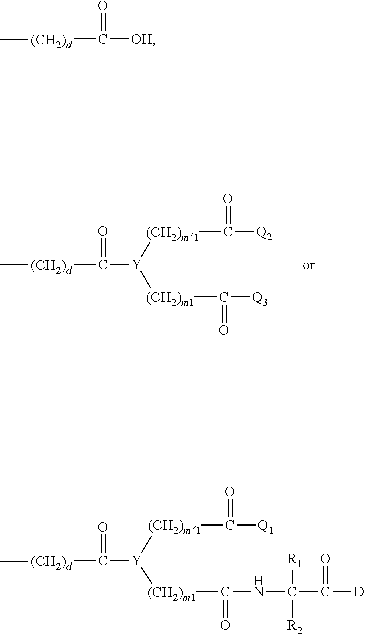

(in this aspect, (q1) and (q2) are both one),
wherein
(d) is zero, 1 or 2; and
at least one or more (e.g., 1, 2, 3, 4, 5, 6, 7, 8) of Z are

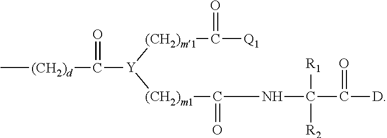

Preferably, Q₁, in each occurrence, is all

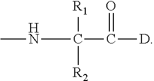

In certain embodiments, (q1) and (q2) are both 1 so that the compounds of Formula (I) include a branching moiety.

In one preferred embodiment, the compounds of Formula (I) include adenine nucleoside analogs (e.g., toyocamycin or analogs) linked via a Z group. Z group is the following:

(i) Z is H,

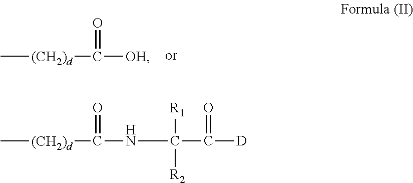

(in this respect, (q) is zero)
wherein (d) is zero, 1 or 2, and at least one or more (e.g., 1, 2, 3, 4, 5, 6, 7, 8) of Z are

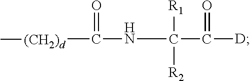

(ii) Z is

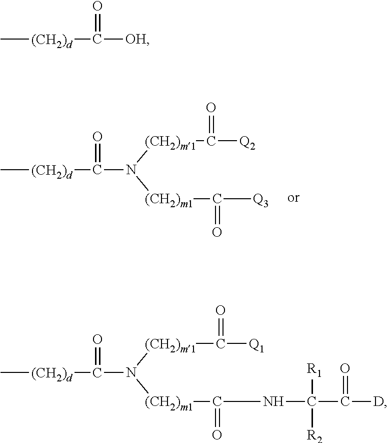

((q1) and (q2) are each 1, and Y is N),
wherein
(d) is zero, 1 or 2;
(m1) and (m′1) are 1 or 2; and
at least one or more (e.g., 1, 2, 3, 4, 5, 6, 7, 8) of Z are

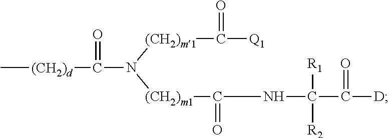

(iii) Z is

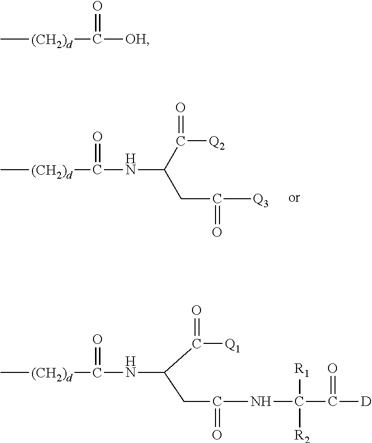

((q1) and (q2) are each 1, and Y is —NH—CH—),
wherein
(d) is zero, 1 or 2; and
at least one or more (e.g., 1, 2, 3, 4, 5, 6, 7, 8) of Z are

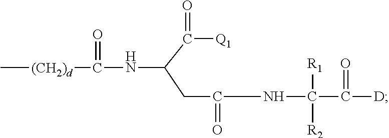

or

(iv) Z is

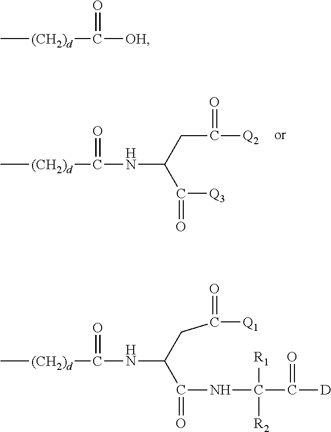

((q1) and (q2) are each 1, and Y is —NH—CH—),
wherein
(d) is zero, 1, or 2; and
at least one or more (e.g., 1, 2, 3, 4, 5, 6, 7, 8) of Z are

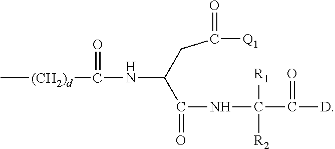

In another preferred embodiment, the compounds described herein are provided in which adenine analogs include 7-deazaadenine nucleosides. In the aspect, Y₁ is O; J₁ is carbon; R_{b1}, in each occurrence, is independently hydrogen, hydroxyl, or —CH₂—OR_{c1}; R_{b2}, in each occurrence, is independently hydrogen or OR_{c2}; R_{b3}, in each occurrence, is independently hydrogen, or OR_{c3}; R_{b4}, in each occurrence, is independently hydrogen, cyano or —C(═O)NH₂; R_{b5} is hydrogen, amine or —NH—NH₂; R_{b6} is hydrogen; and R_{c1}, R_{c2}, and R_{c3}, in each occurrence, are independently hydrogen, acyl, monophosphate, diphosphate, or triphosphate.

In another preferred embodiment, the present invention is provided in which R_{b1}, in each occurrence, is —CH₂—OR_{c1}, R_{c1} is hydrogen, monophosphate, diphosphate, or triphosphate; R_{b2}, and R_{b3}, in each occurrence, are both hydroxyl; R_{b4}, in each occurrence, is independently cyano or —C(═O)NH₂; and R_{b5} and R_{b6} are both hydrogen.

In yet another preferred embodiment, the compounds described herein are provided in which Y₁ is O; J₁ is N; R_{b1}, in each occurrence, is —CH₂—OR_{c1}, wherein R_{c1} is hydrogen, monophosphate, diphosphate, or triphosphate; R_{b2}, in each occurrence, is independently hydrogen or hydroxyl; R_{b3}, in each occurrence, is independently hydrogen, hydroxyl or F; and R_{b5} is hydrogen; and R_{b6} is hydrogen, F, or Cl.

It shall be understood that at least one arm of the compound of Formula (I) includes D, i.e., an adenine nucleoside or a derivative thereof (e.g., toyocamycin, sangivamycin, or 5′-mono-, di, tri-phosphate thereof).

According to the present invention, adenine nucleoside analogs (e.g., toyocamycin and analogs) are attached at the amine to each polymer arm via a spacer containing R₁ and R₂.

In certain embodiments, R₁, in each occurrence, is independently H, C₁₋₆ alkyl, C₃₋₆ branched alkyl, or C₃₋₆ cycloalkyl; and R₂, in each occurrence, is independently C₁₋₆ alkyl, C₃₋₆ branched alky or C₃₋₆ cycloalkyl. In one embodiment, the compounds described herein are provided in which R₁ is hydrogen, methyl, ethyl, propyl, butyl, isobutyl, cyclopropyl, cyclobutyl, cyclopentyl, or cyclohexyl, and R₂ is methyl, ethyl, propyl, butyl, isobutyl, cyclopropyl, cyclobutyl, cyclopentyl, or cyclohexyl. In this respect, the combination of R₁ and R₂ contemplated according to Formula (I) includes, but is not limited to, hydrogen/methyl, hydrogen/ethyl, hydrogen/propyl, hydrogen/isopropyl, hydrogen/butyl, hydrogen/isobutyl, and so forth. Likewise, the combination includes methyl/methyl, methyl/ethyl, and so forth. In one preferred embodiment, R₁ is hydrogen and R₂ is isobutyl, or R₁ and R₂ are both methyl.

In one preferred aspect of the present invention, the compounds of Formula (I) include a Z group in which a toyocamycin (i.e., toyocamycin and sangivamycin) taken in combination with a spacer has the structure:

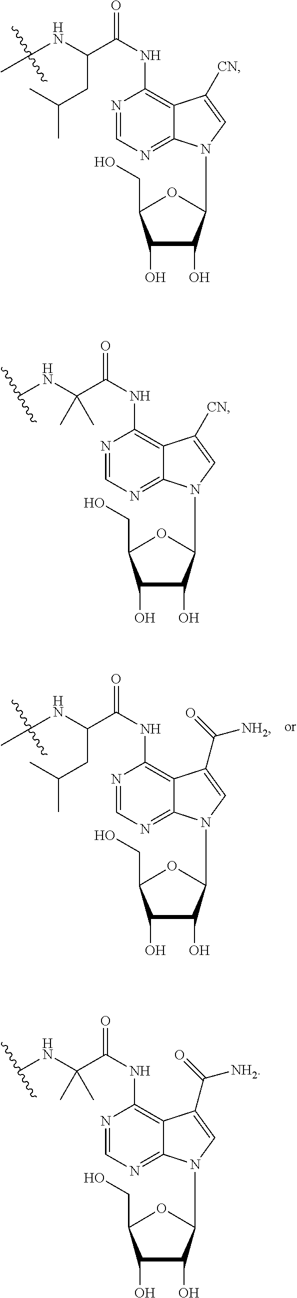

In another preferred aspect of the present invention, the compounds of Formula (I) include multi-armed polymers (e.g., four-arm PEGs and eight-arm PEGs). One preferred aspect of the present invention provides compounds having the formula:

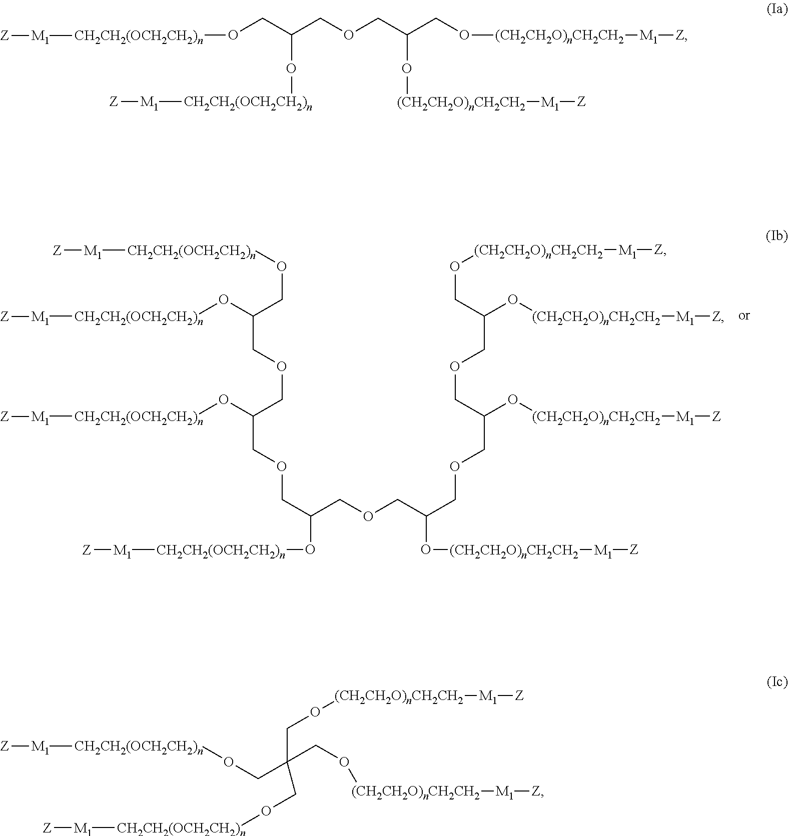

wherein

M₁ is independently O, or S;

Z is one of the following:

(i) Z is independently H,

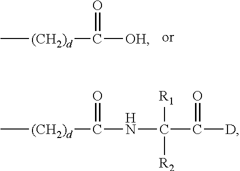

wherein (d) is zero, 1 or 2,
provided that one or more (e.g., 1, 2, 3, 4, 5, 6, 7, 8, preferably 4 and 8 of four and 8 polymer arms) of Z groups are

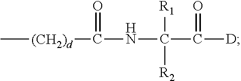

(ii) Z is

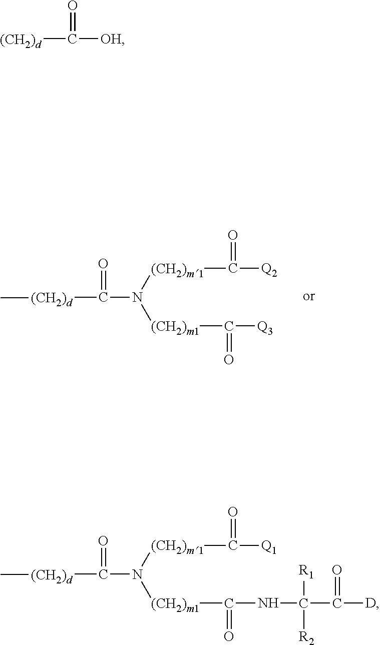

wherein
(d) is zero, 1 or 2;
(m) and (m′) are 1 or 2; and
Q₁, Q₂ and Q₃, in each occurrence, are independently OH or

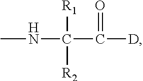

provided that one or more (e.g., 1, 2, 3, 4, 5, 6, 7, 8, preferably 4 and 8 of 4 and 8 polymer arms) of Z groups are

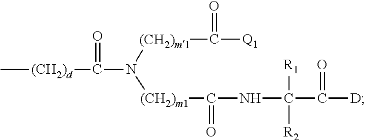

(iii) Z is

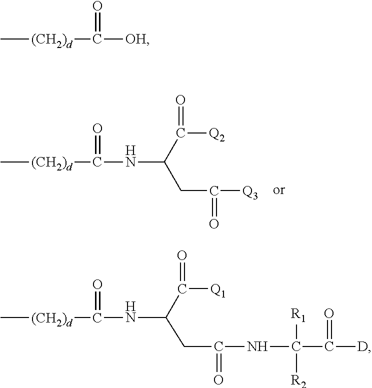

wherein
(m) is all 1;
(m′) are all zero;
(d) is zero, 1 or 2; and
Q₁, Q₂ and Q₃, in each occurrence, are independently OH or

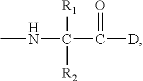

provided that one or more (e.g., 1, 2, 3, 4, 5, 6, 7, 8, preferably 4 and 8 of 4 and 8 polymer arms) of Z groups are

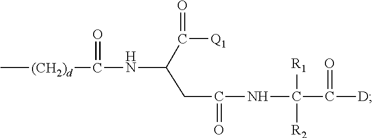

or

(iv) Z is

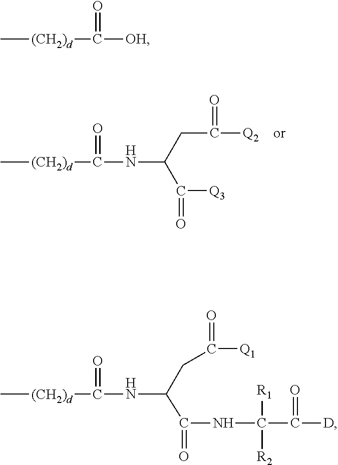

wherein
(m) is all 0;
(m′) are all 1;
(d) is zero, 1 or 2;
Q₁, Q₂ and Q₃, in each occurrence, are independently OH or

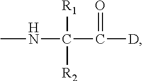

provided that one or more (e.g., 1, 2, 3, 4, 5, 6, 7, 8, preferably 4 and 8 of 4 and 8 polymer arms) of Z groups are

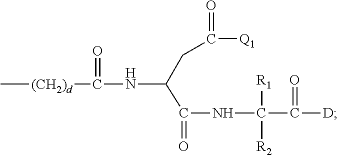

R₁, in each occurrence, is independently H, C₁₋₁₀ alkyl, C₃₋₁₀ branched alkyl, C₃₋₈ cyclicalkyl, C₂₋₁₀ alkenyl;

R₂, in each occurrence, is independently C₁₋₁₀ alkyl, C₃₋₁₀ branched alkyl, C₃₋₈ cyclicalkyl, C₂₋₁₀ alkenyl; and

D is

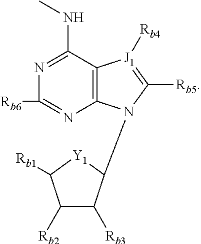

Preferably, the multi-arm compounds described herein are provided in which D is

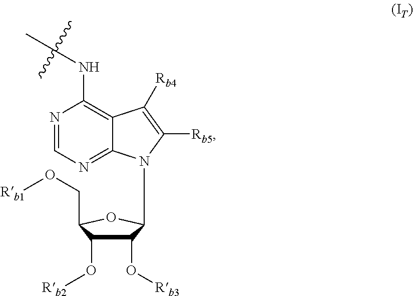

wherein

R′_{b1}, R′_{b2} and R′_{b3} are independently hydrogen, monophosphate, diphosphate, or triphosphate;

R_{b4} is —CN, —C(═O)NH₂, or hydrogen; and

R_{b5} is hydrogen, amine, or —NH—NH₂,

or a pharmaceutical salt thereof.

In this aspect, (n) is independently a positive integer of from about 10 to about 2,300 so that the total average molecular weight of the polymeric portion of the compound of ranges from about 2,000 to about 100,000 daltons.

In this aspect and in some embodiments, R₁, in each occurrence, is independently H, C₁₋₆ alkyl, C₃₋₆ branched alkyl, or C₃₋₆ cycloalkyl. R₂, in each occurrence, is independently C₁₋₆ alkyl, C₃₋₆ branched alky or C₃₋₆ cycloalkyl. In an alternative embodiment, R₁ is hydrogen, methyl, ethyl, propyl, isopropyl butyl, isobutyl, cyclopropyl, cyclobutyl, cyclopentyl, or cyclohexyl, and R₂ is methyl, ethyl, propyl, butyl, isobutyl, cyclopropyl, cyclobutyl, cyclopentyl, or cyclohexyl. In this respect, the combination of R₁ and R₂ contemplated according to Formula (I) includes, but is not limited to, hydrogen/methyl, hydrogen/ethyl, hydrogen/propyl, hydrogen/isopropyl, hydrogen/butyl, hydrogen/isobutyl, and so forth. Likewise, the combination includes methyl/methyl, methyl/ethyl, and so forth. In one preferred embodiment, R₁ is hydrogen and R₂ is isobutyl, or R₁ and R₂ are both methyl.

In one preferred embodiment, R′_{b1} is hydrogen, monophosphate, diphosphate, or triphosphate; R′_{b2} and R′_{b3} are hydrogen.

In another preferred aspect, (n) is an integer of from about 28 to about 341, so that the total average number molecular weight of the polymeric portion of the compounds described herein ranges from about 5,000 to about 60,000 daltons. In one alternative preferred embodiment, (n) is an integer of from about 114 to about 239, so that the total molecular weight of the polymeric portion of the compounds of Formula (I) ranges from about 20,000 to about 42,000 daltons.

The present invention provides four-arm PEG conjugates of adenine nucleoside analogs. The conjugates contemplated include:

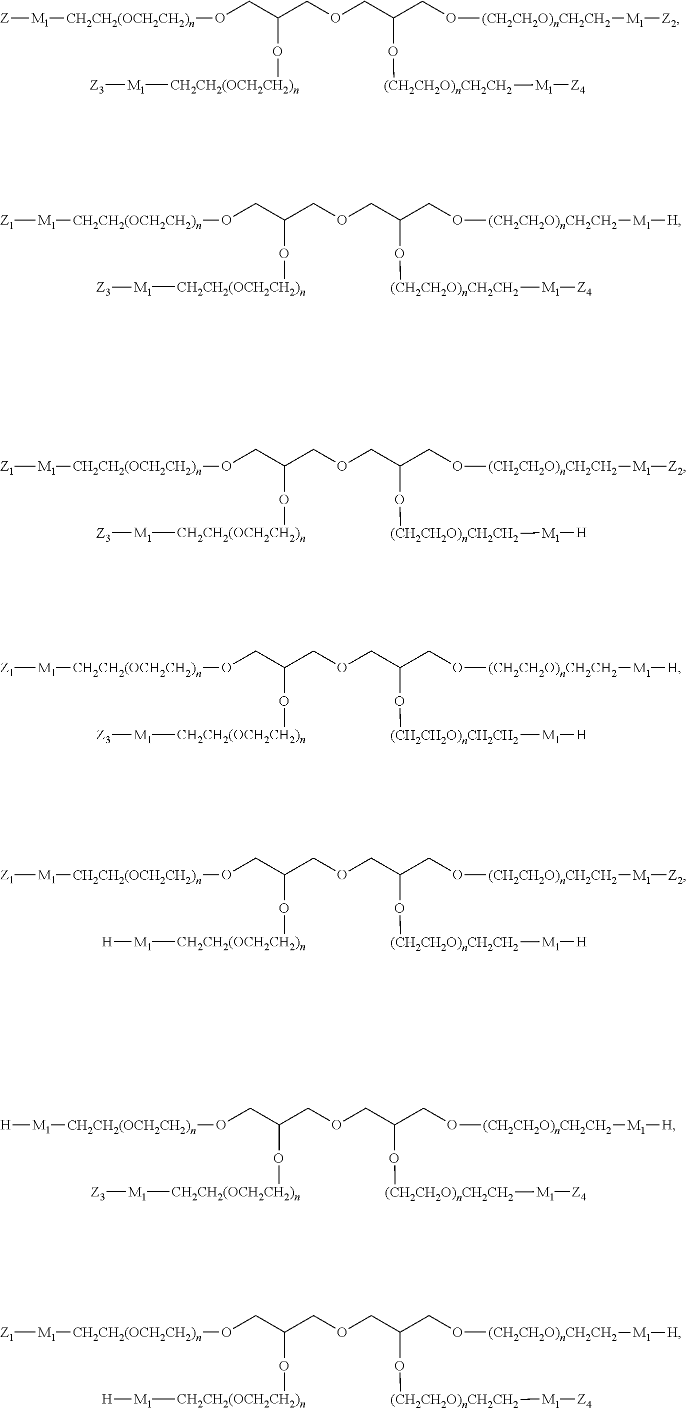

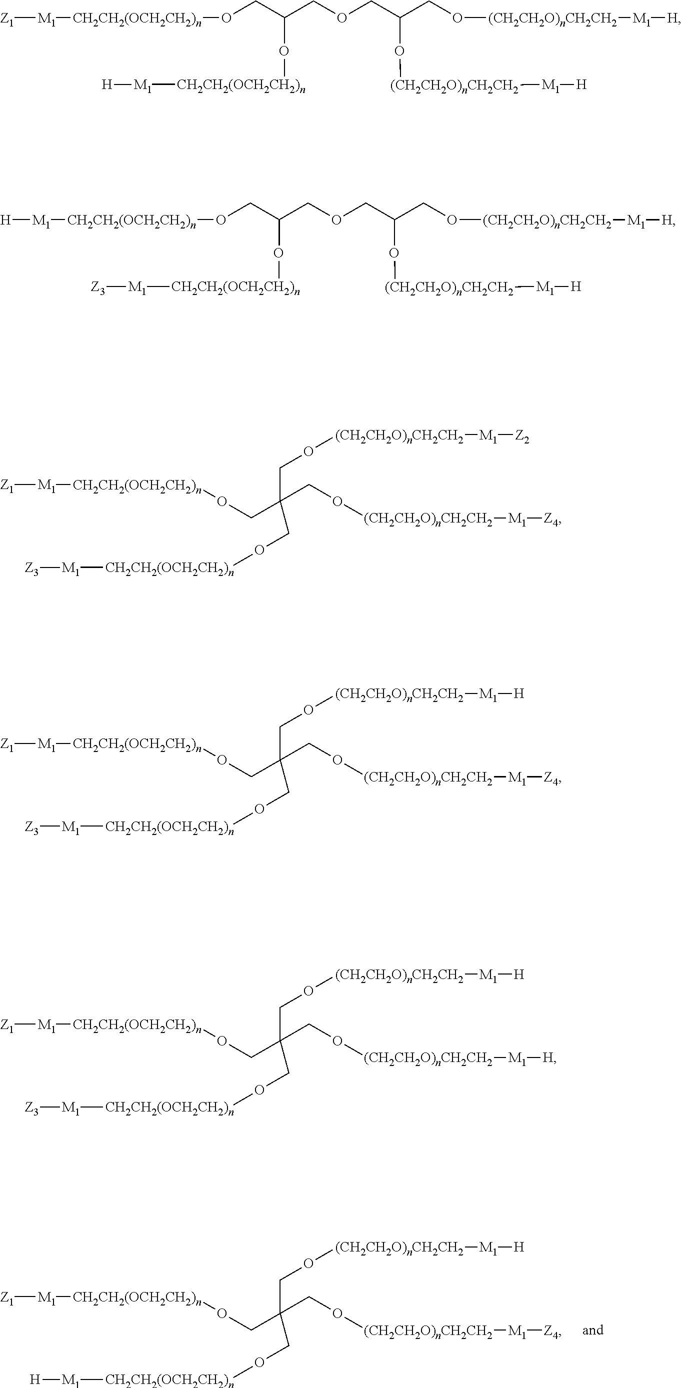

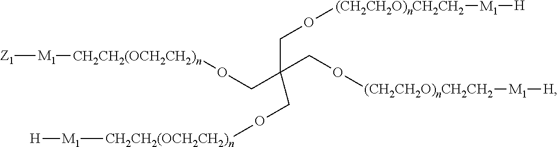

wherein

Z₁, Z₂, Z₃, and Z₄ are all

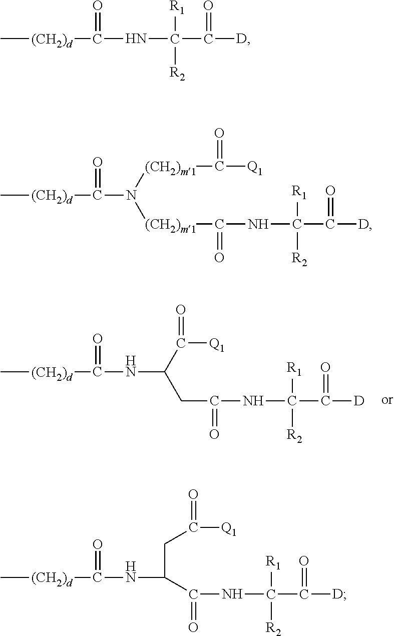

wherein

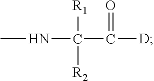

Q₁ is hydroxyl or
(d) is zero, 1 or 2;
(n) is a positive integer of from about 10 to about 2,300 so that the polymeric portion of the compound has the total number average molecular weight of from about 2,000 to about 100,000 daltons; and
all other variables are as previously defined.

Some preferred compounds of the invention include:

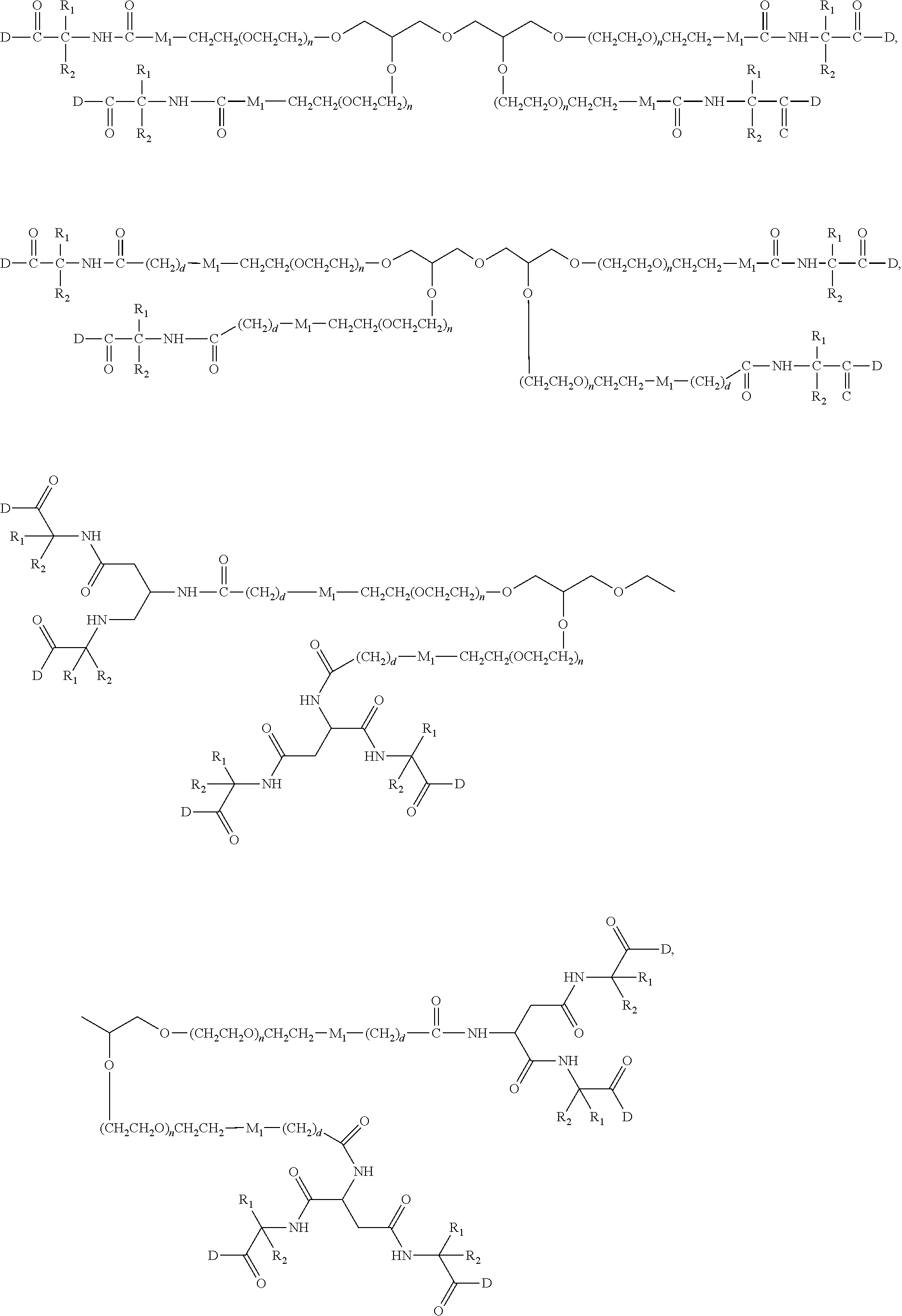

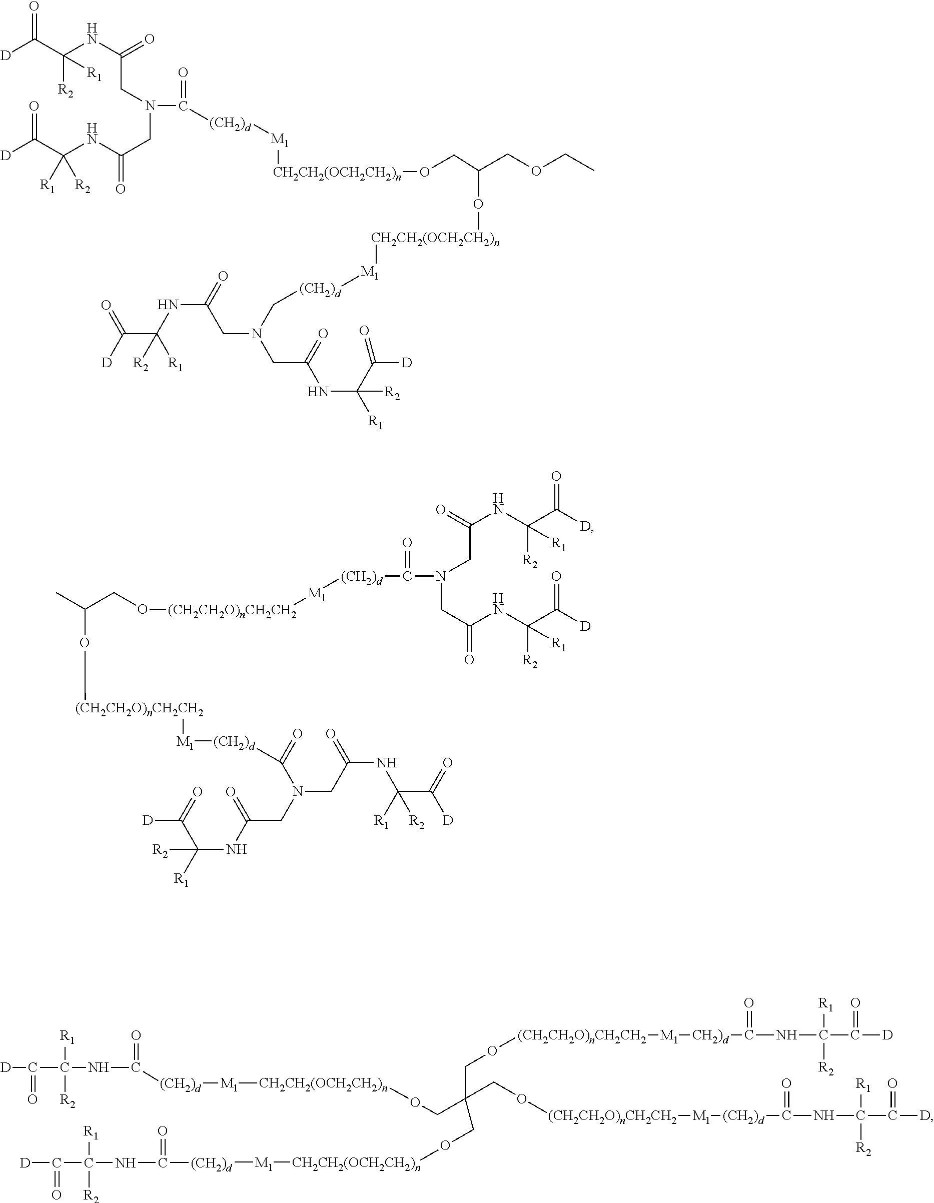

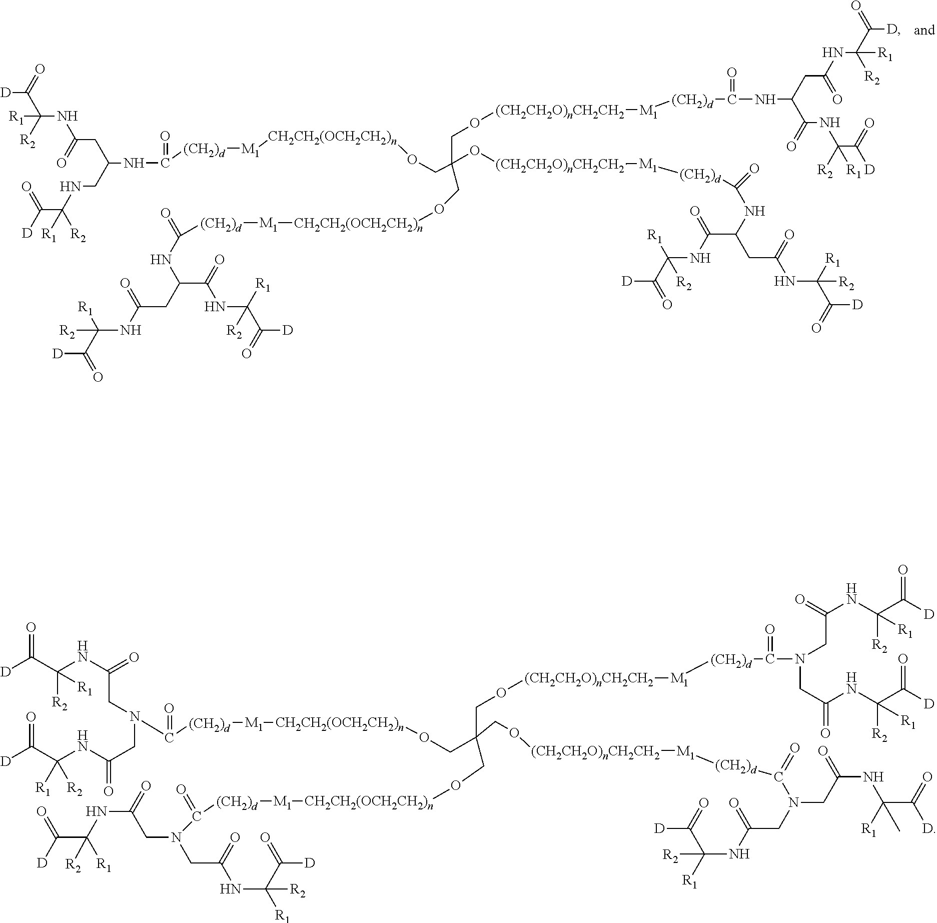

In certain embodiments, partial loadings of adenine nucleoside analogs may occur to provide the following:

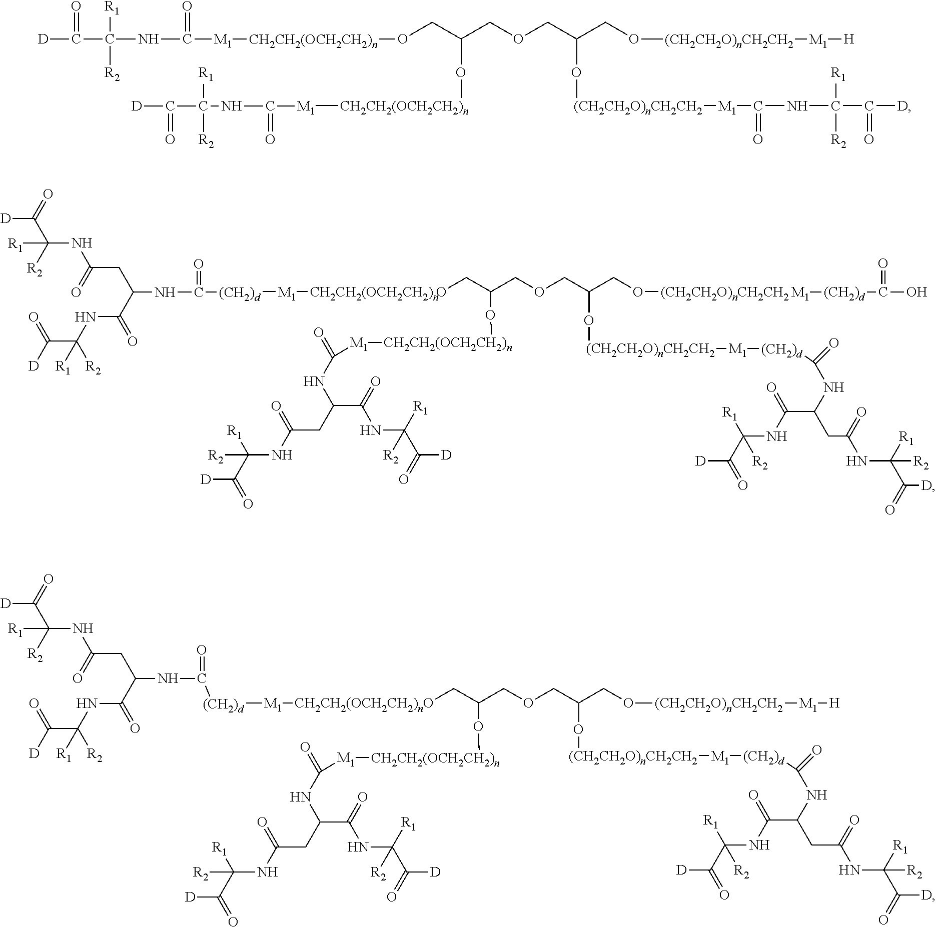

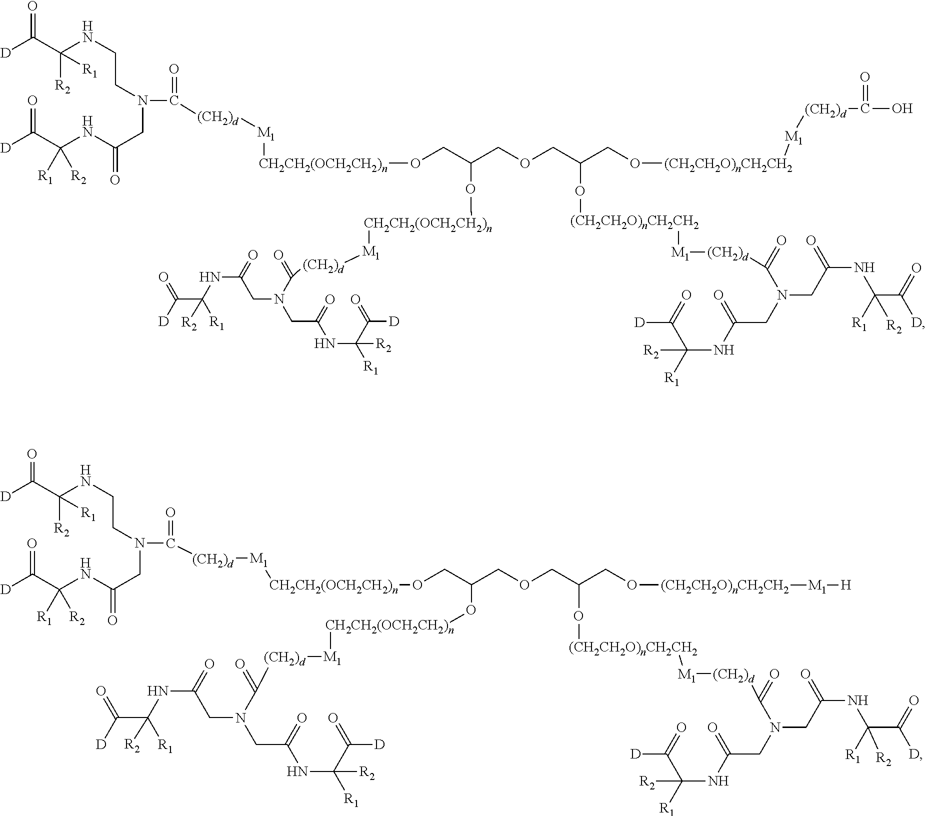

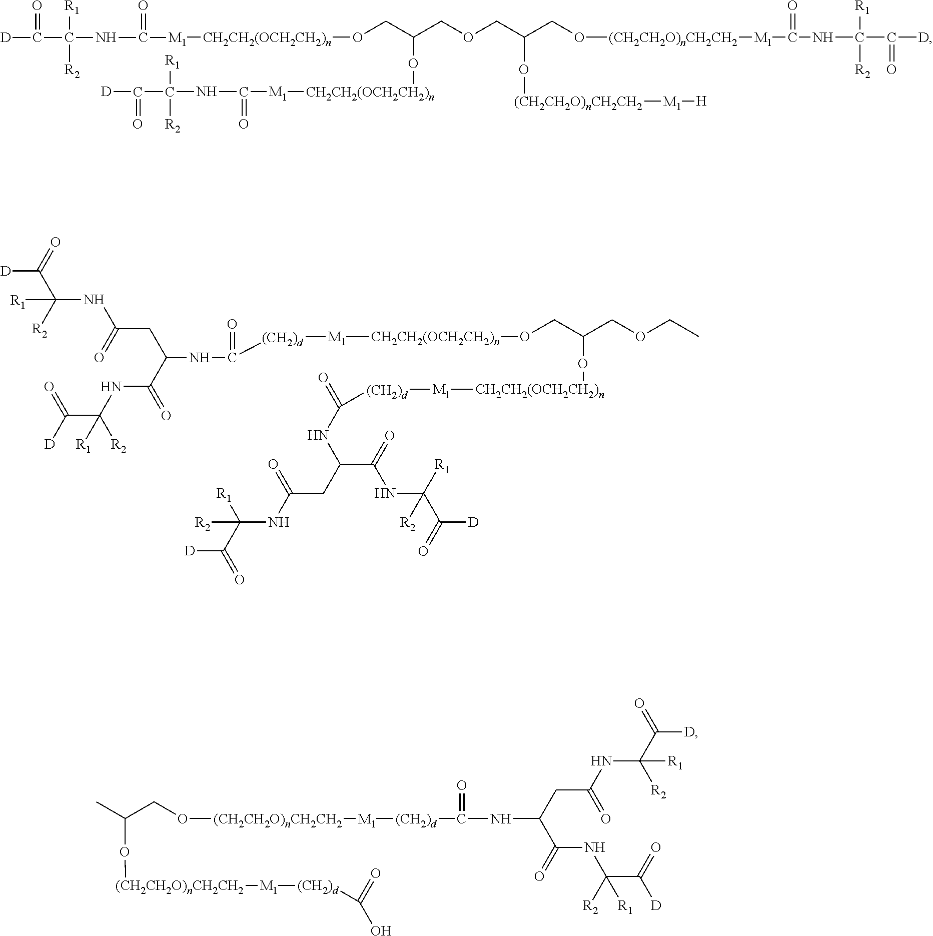

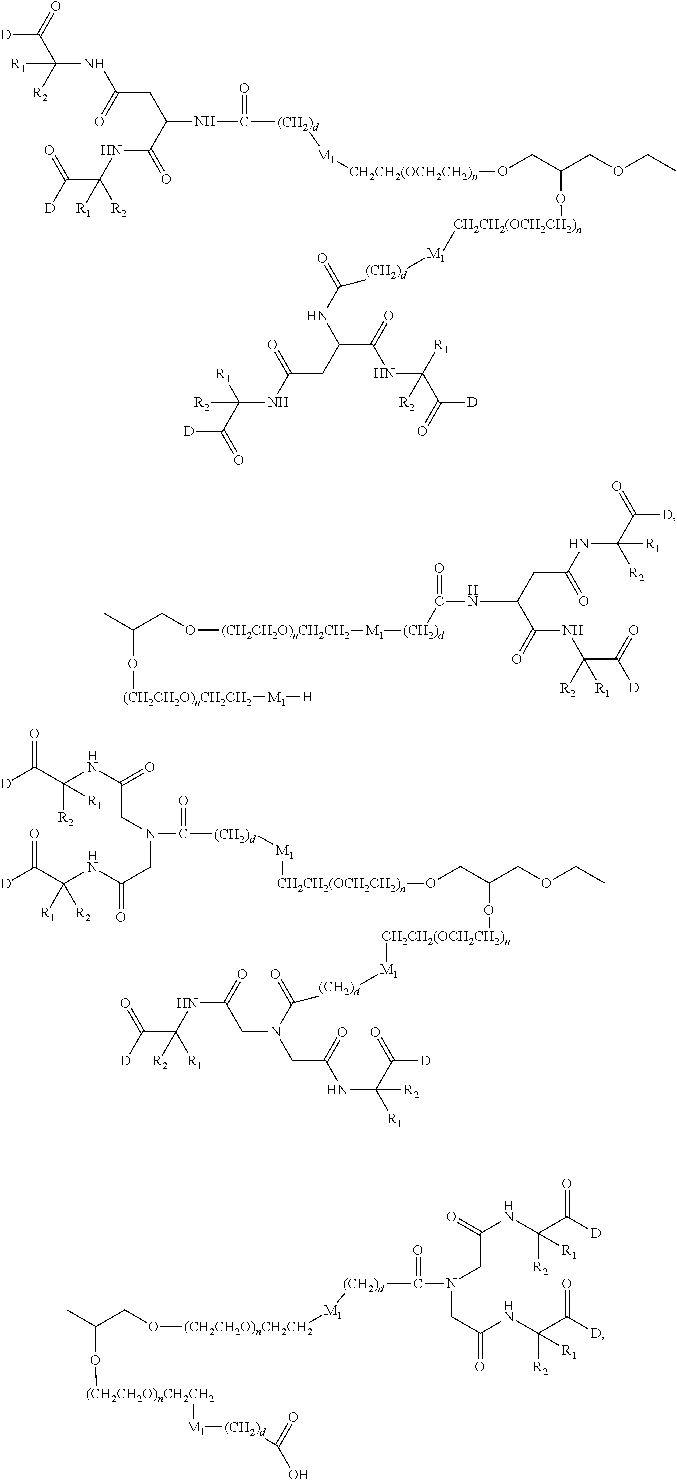

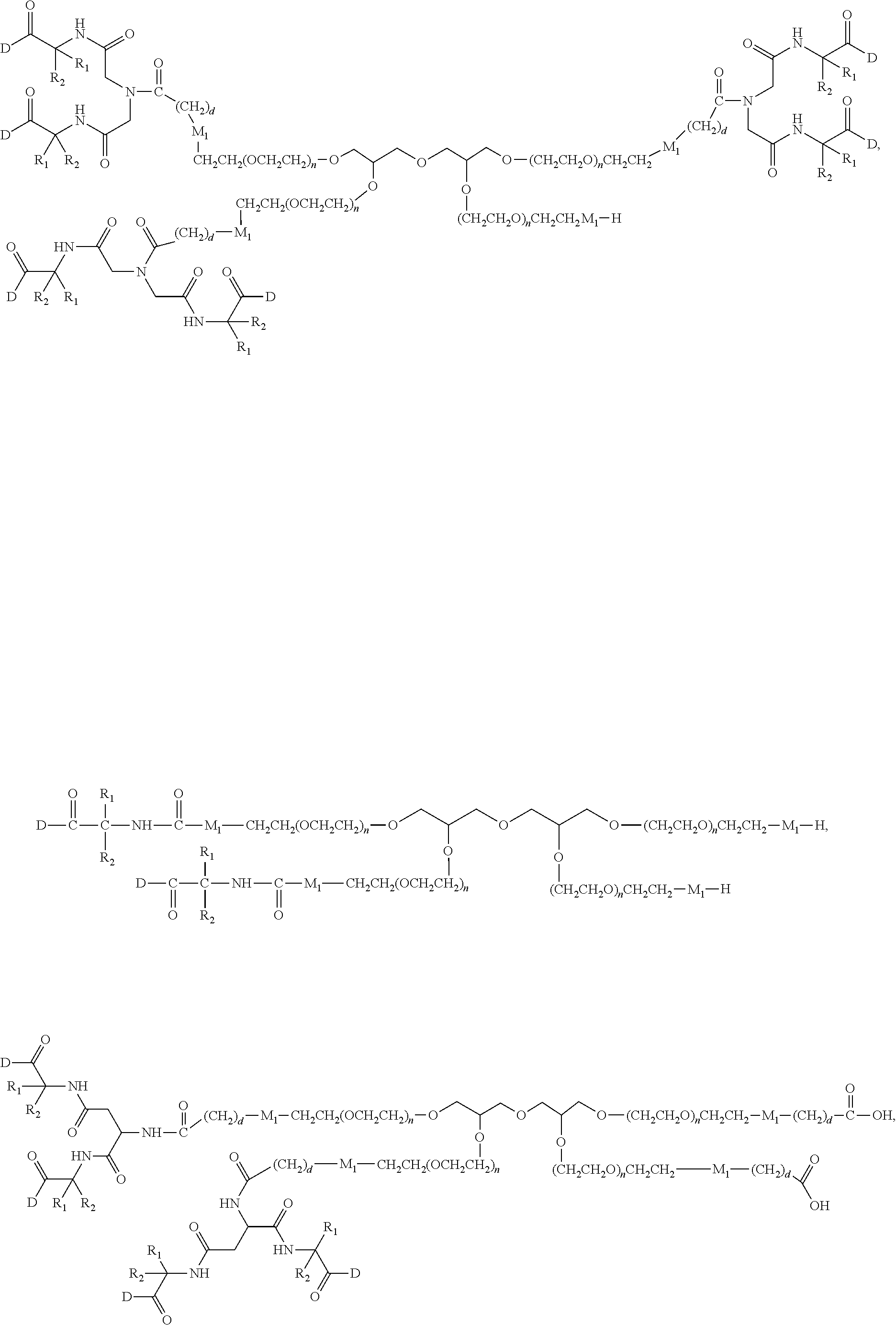

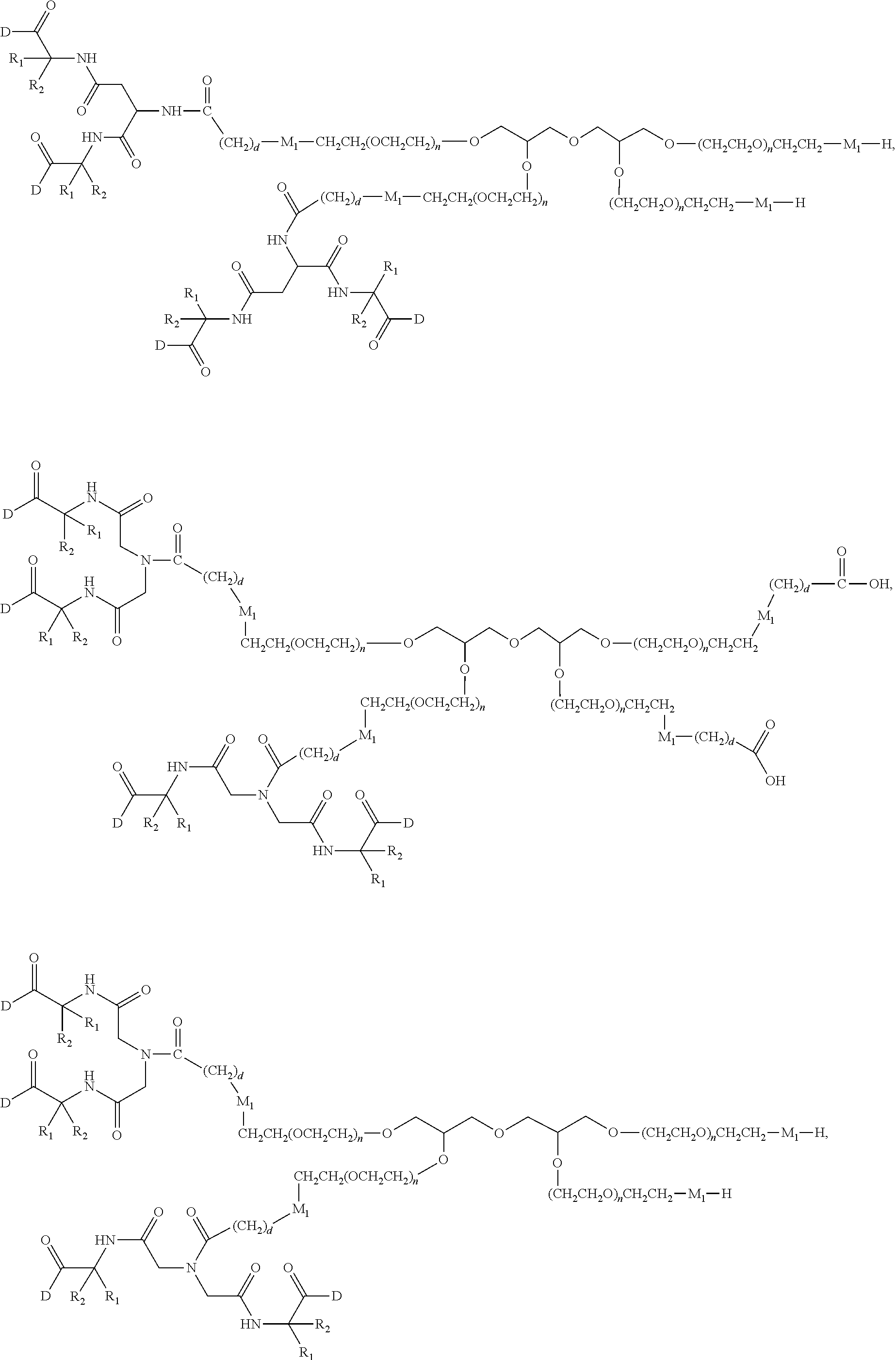

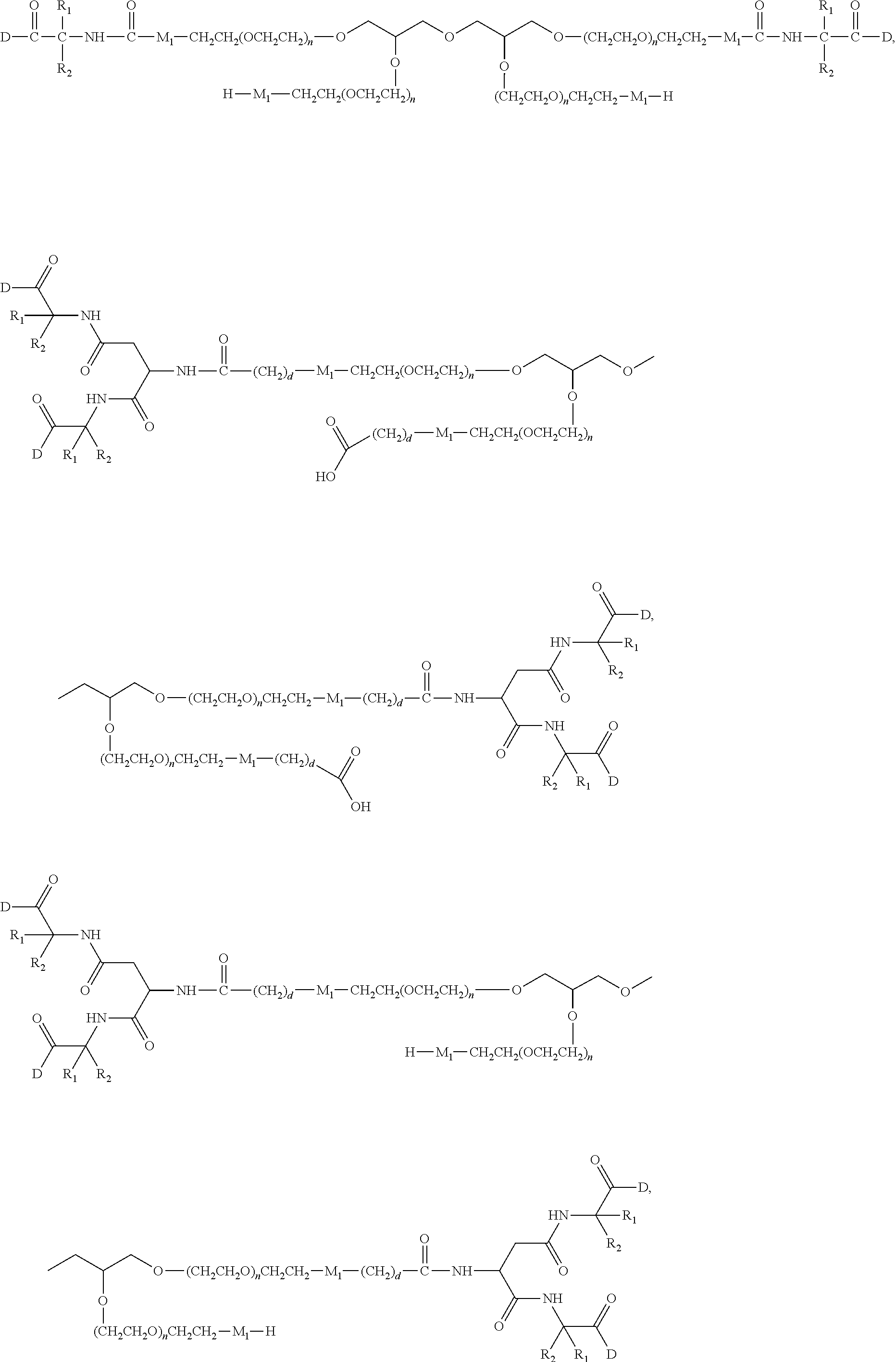

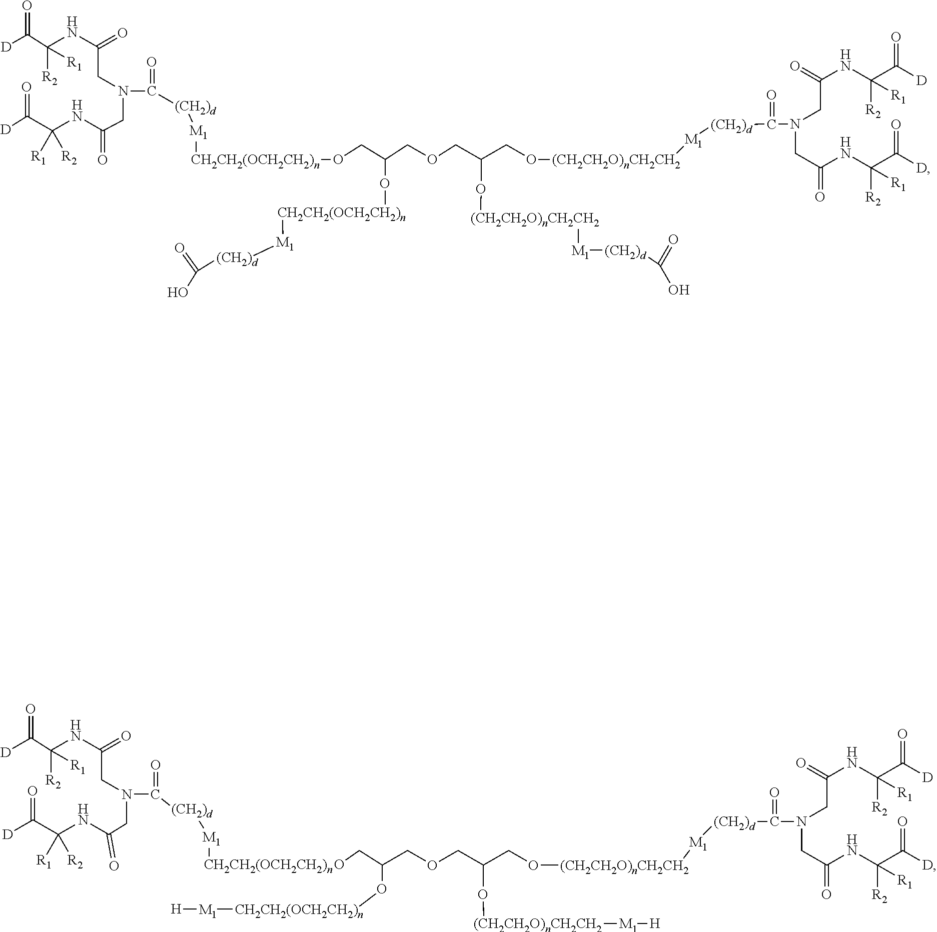

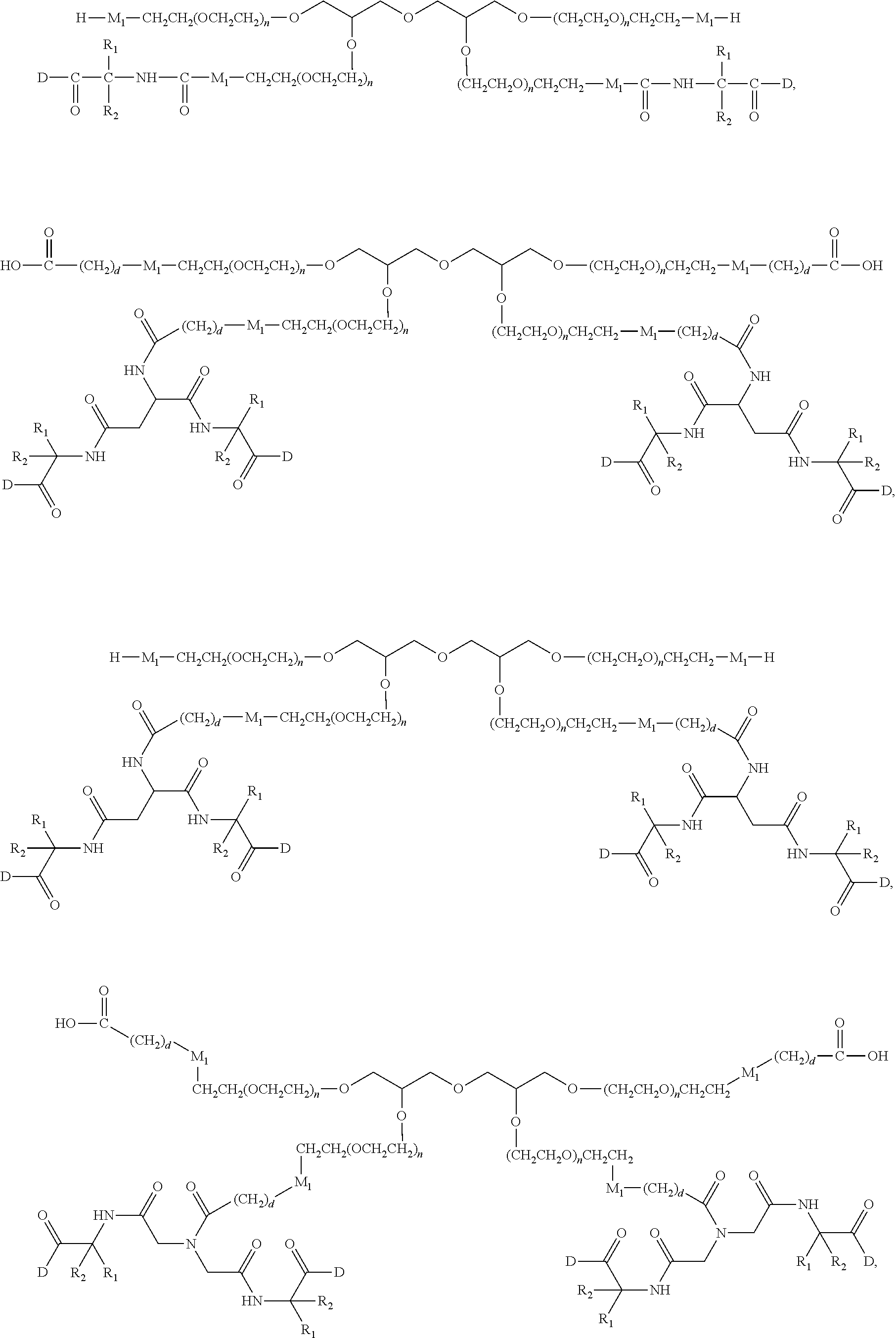

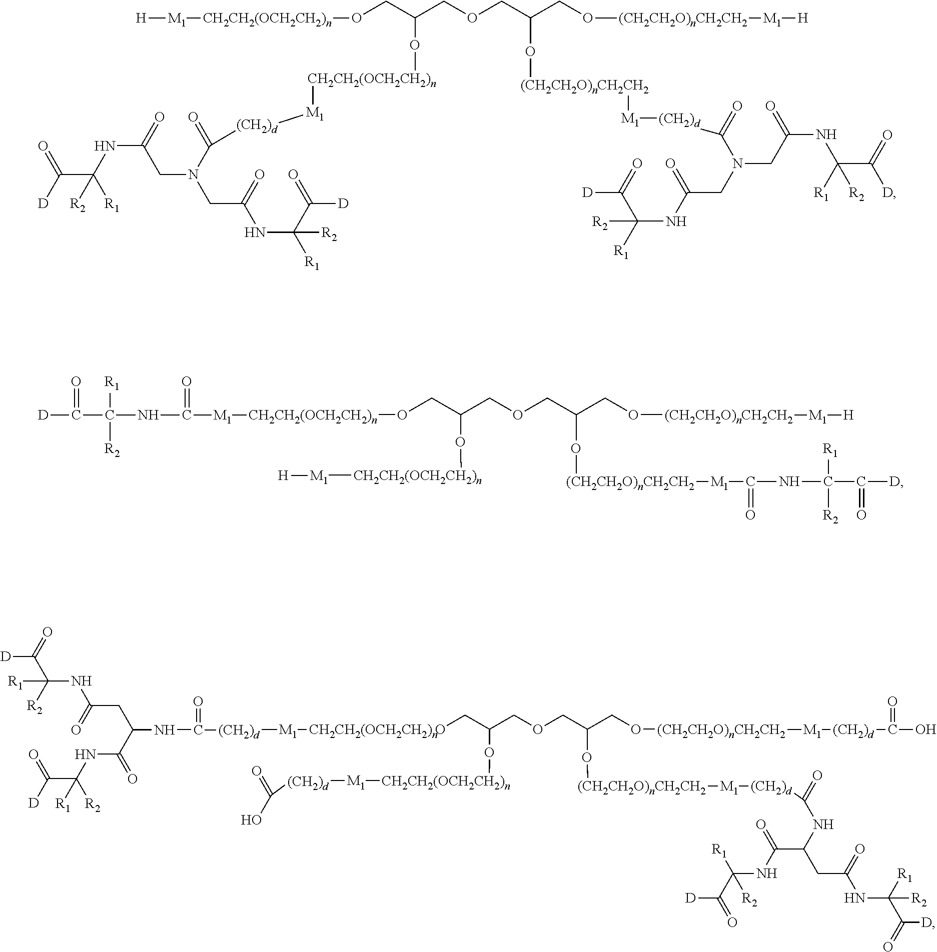

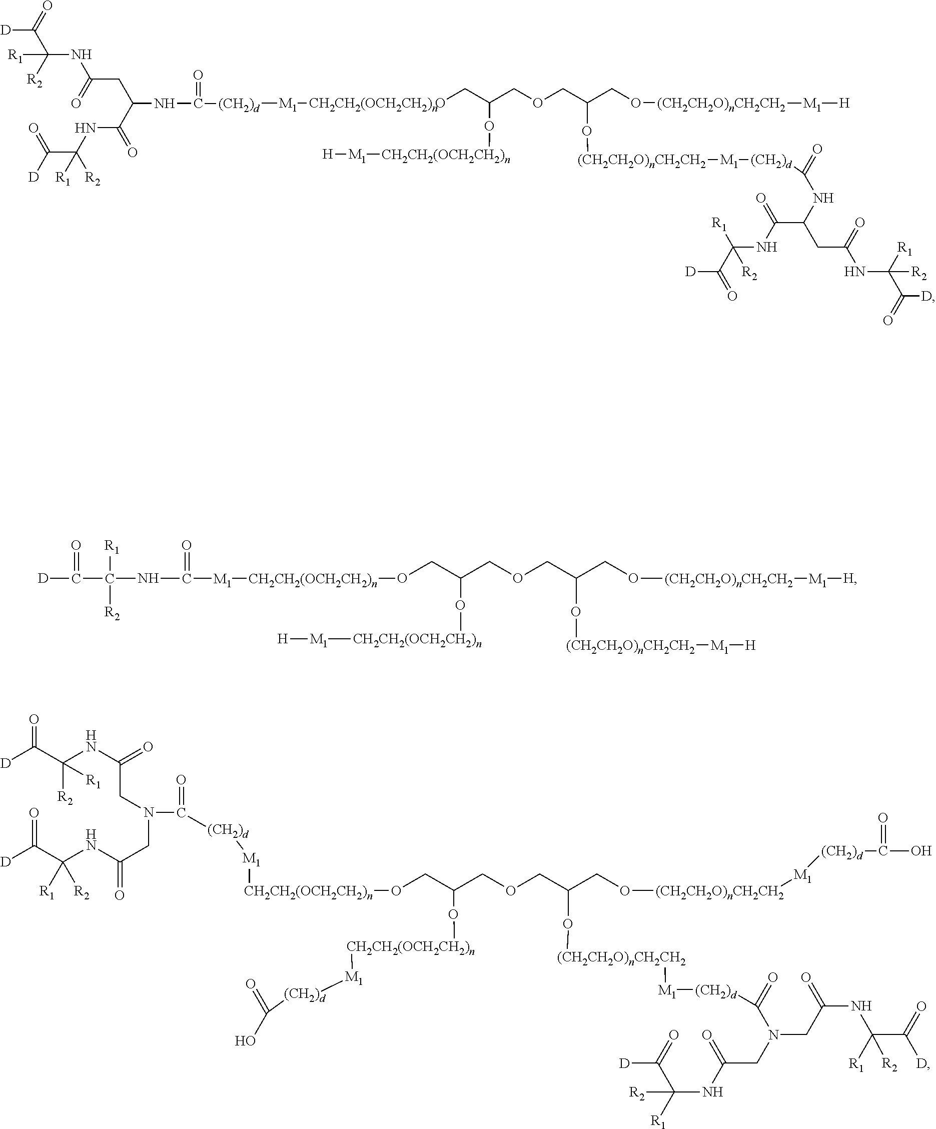

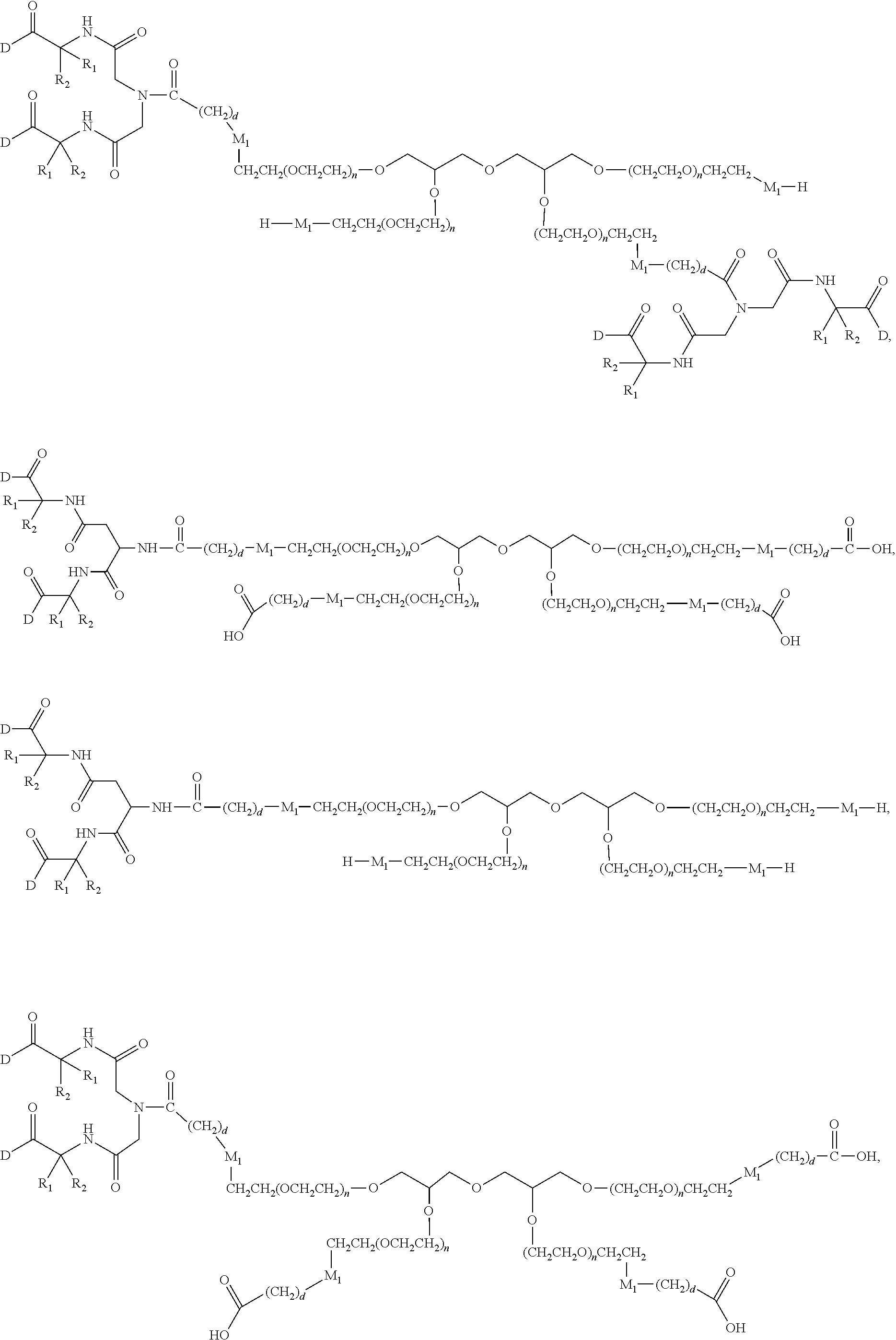

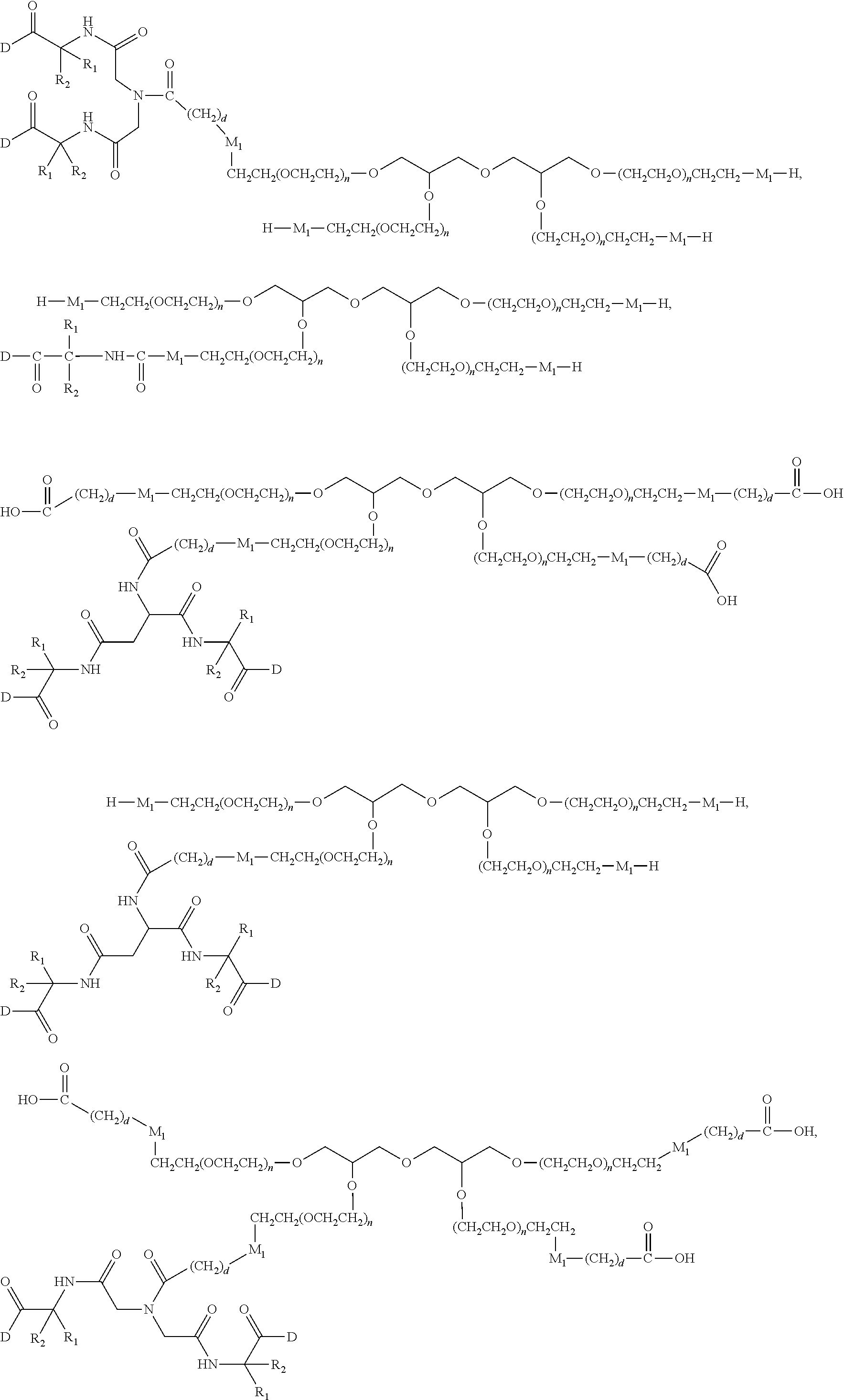

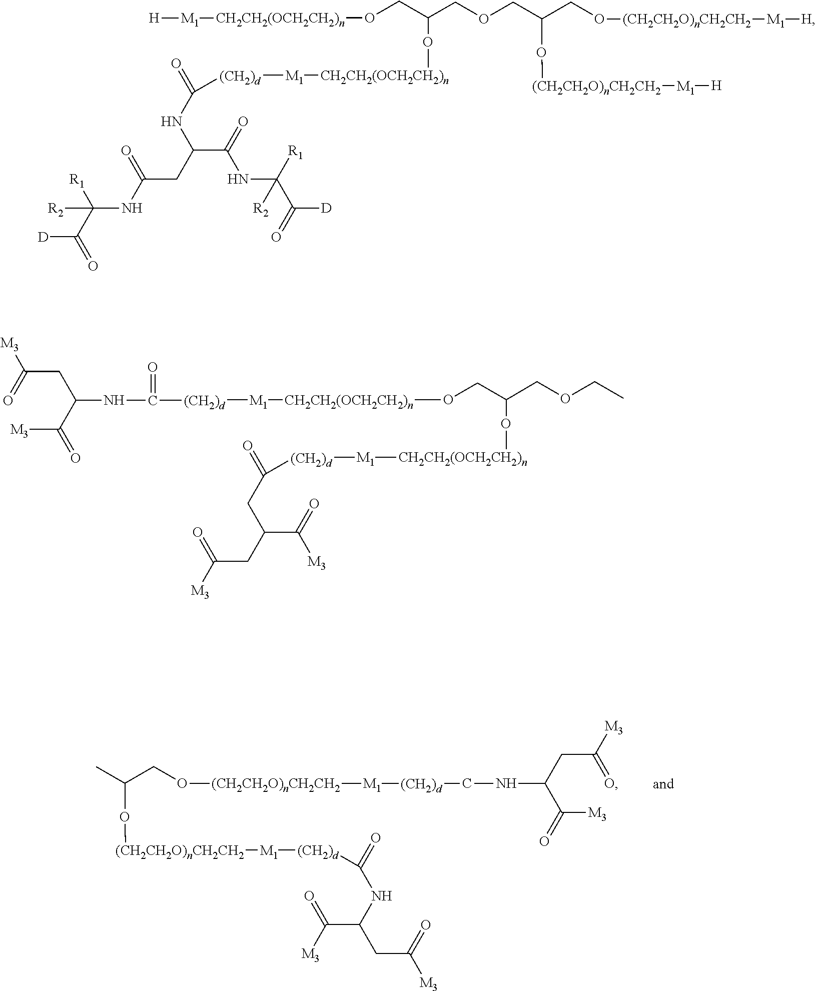

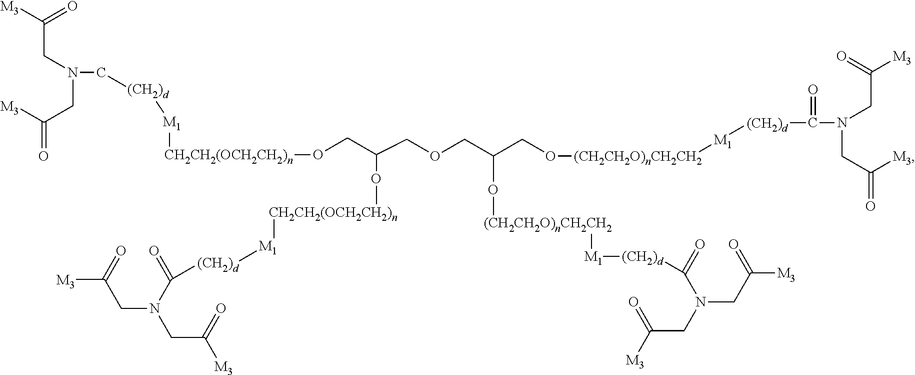

wherein

(d) is zero, 1 or 2;

M₃, in each occurrence, is independently OH, or

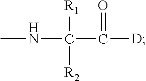

D is an adenosine nucleoside analog (preferably, toyocamycin or an analog thereof; and,

provided that one or more (e.g., 1, 2, 3, 4, 5, 6, 7, 8, preferably 8) of M₃ is

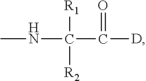

and D is as previously defined.

One preferred embodiment of the present invention includes compound selected from among:

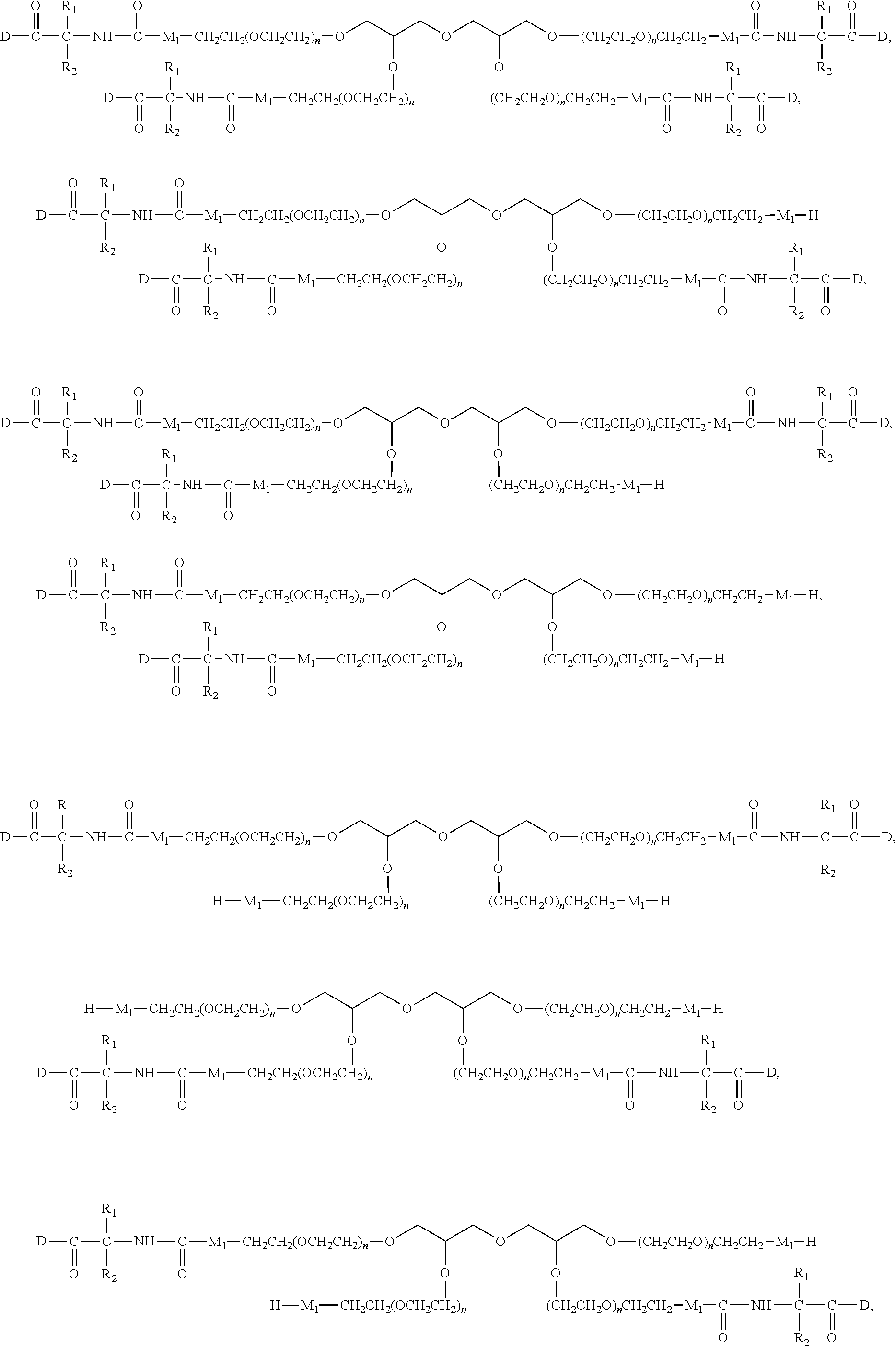

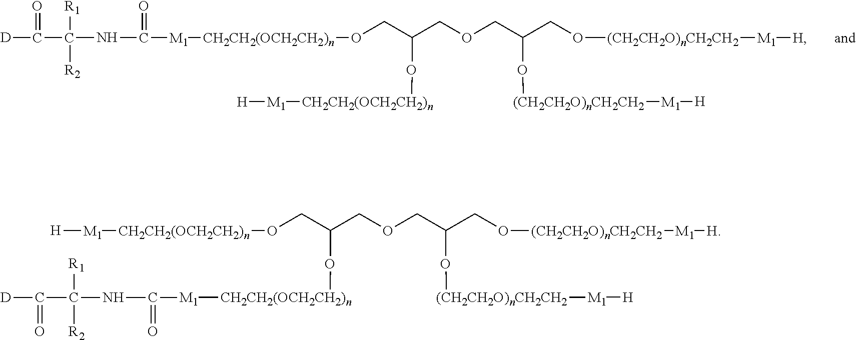

In certain embodiments, the four-arm polymeric conjugates include D:

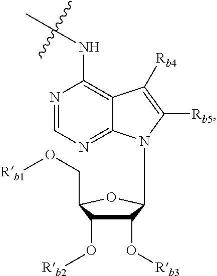

wherein R′_{b1} is hydrogen, monophosphate, diphosphate, or triphosphate;
R′_{b2} and R′_{b3} are hydrogen; and
R_{b4} is —CN or —C(═O)NH₂;
   or a pharmaceutical salt thereof.

In another preferred embodiment, the compounds described herein have the structure:

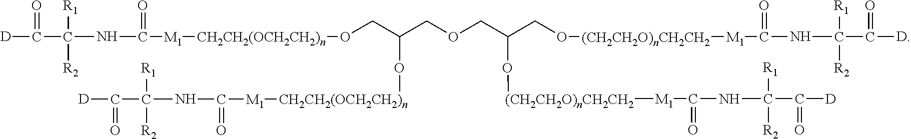

In another preferred embodiment, the drug (D), when taken together with —NH—C(R₁)(R₂)—C(═O)—, forms:

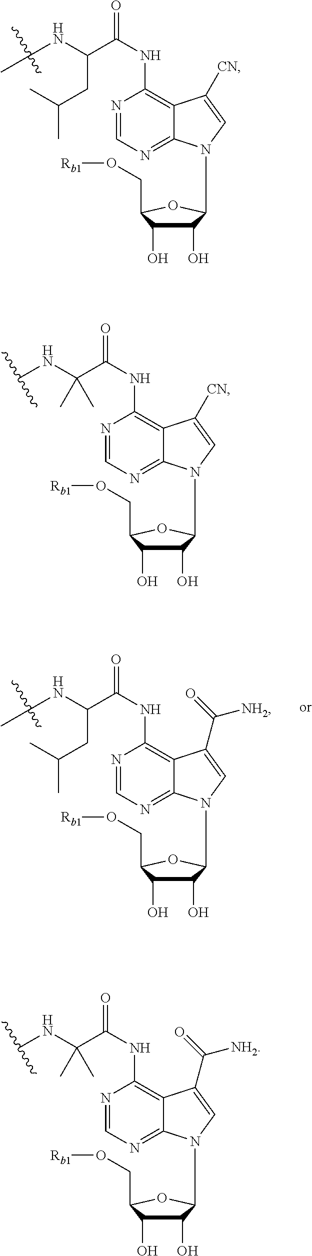

wherein R_{b1} is hydrogen, monophosphate, diphosphate, or triphosphate.

In further embodiments, the drug, when taken together with —NH—C(R₁)(R₂)—C(═O)—, forms:

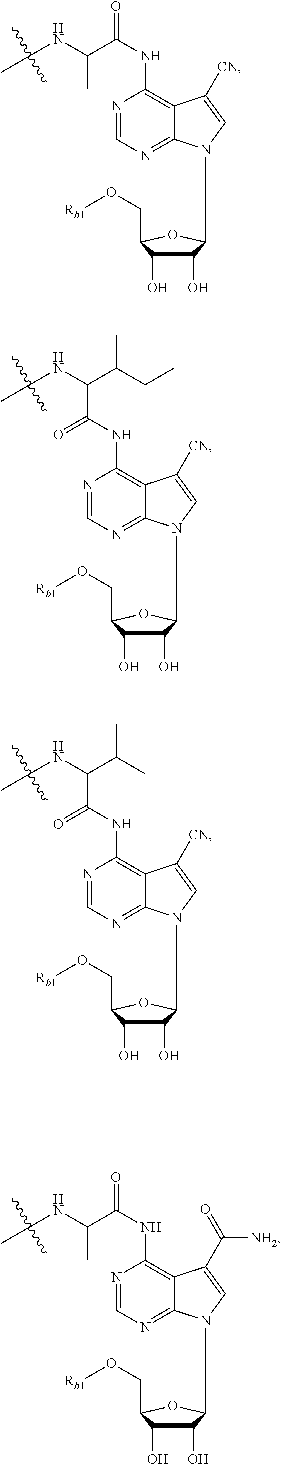

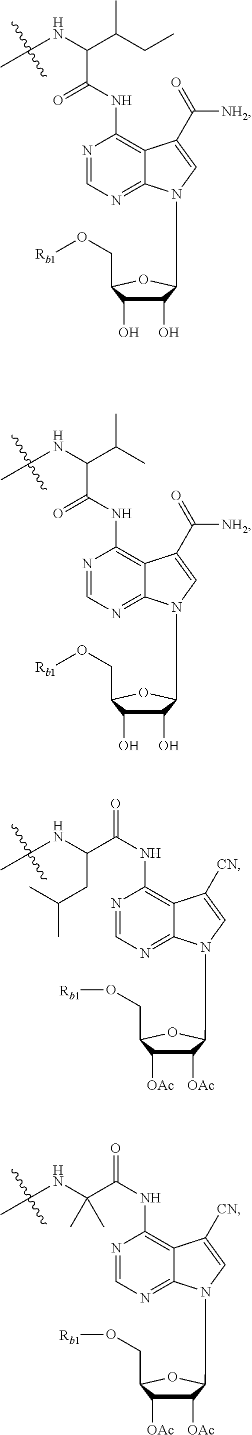

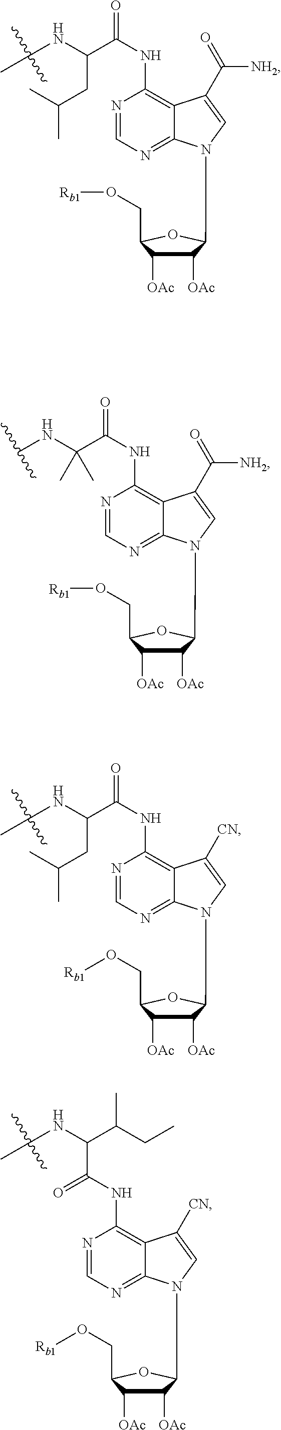

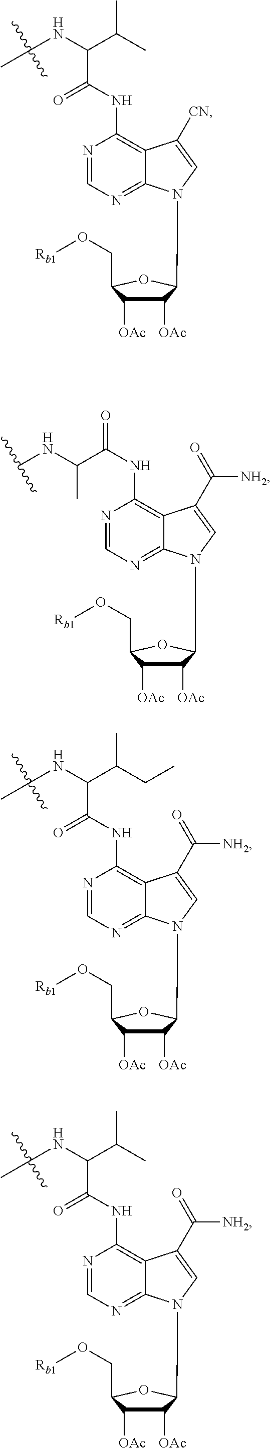

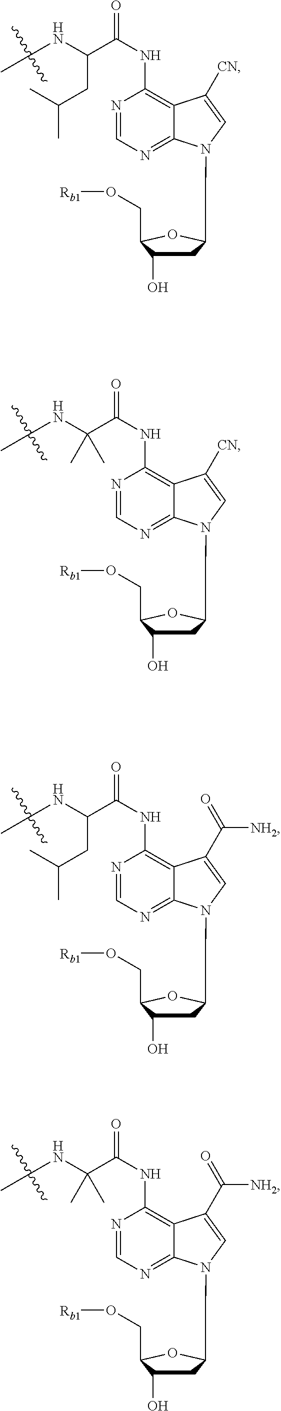

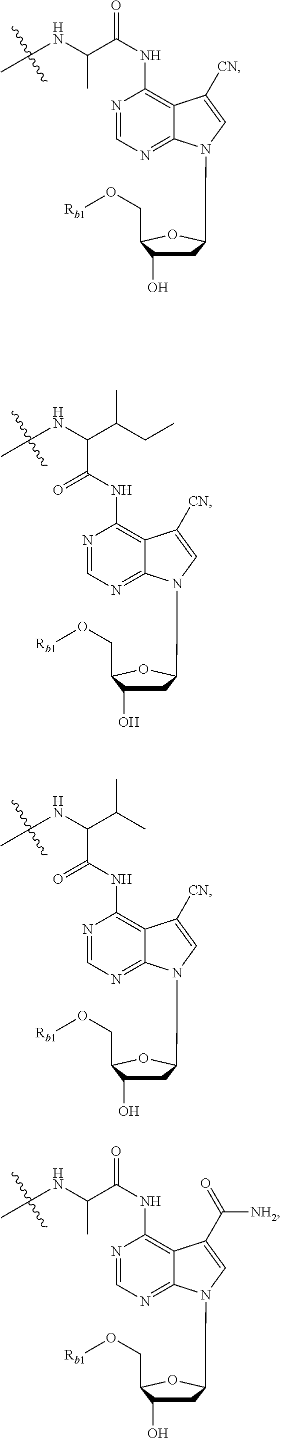

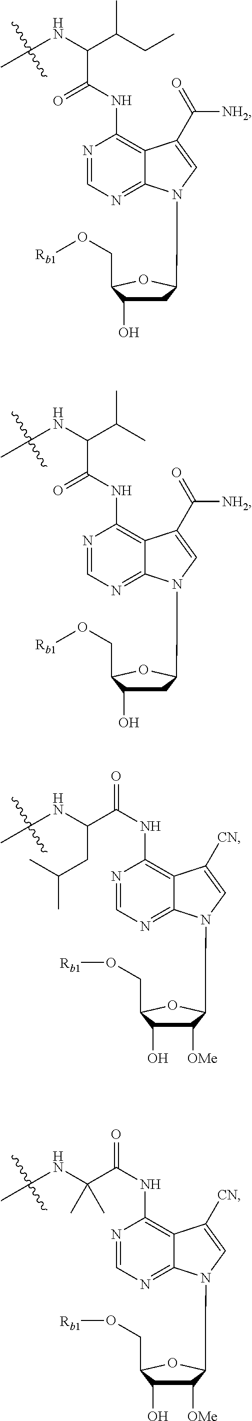

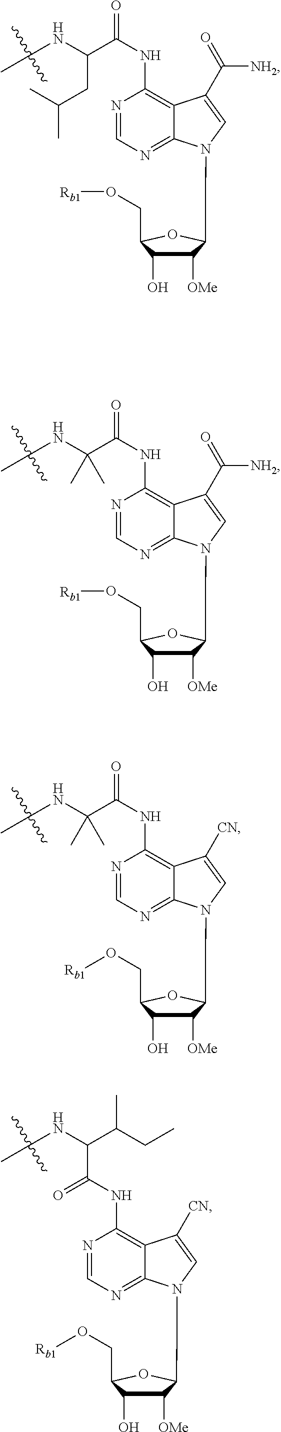

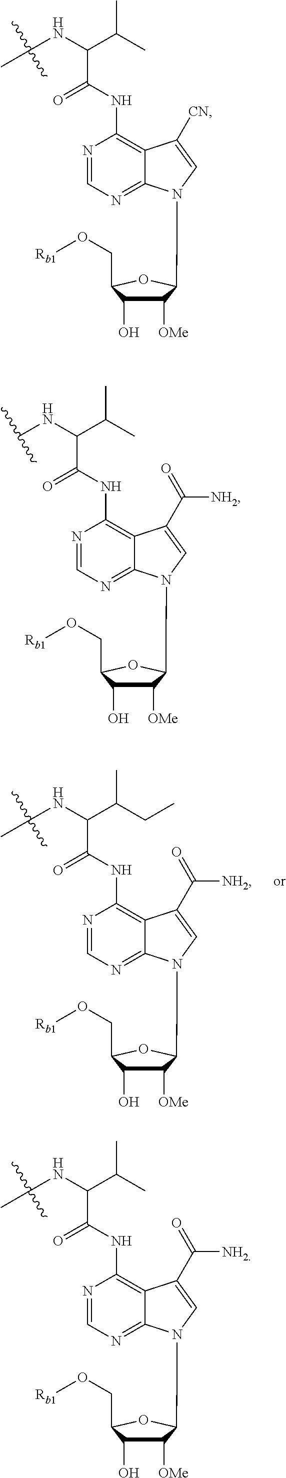

For purposes of the present invention, the combinations of the spacers and branching groups contemplated within the scope of the present invention include those in which combinations of variables of such groups are permissible so that such combinations result in stable compounds of Formula (I).

### B. Non-Antigenic Polymers

A further aspect of the invention provides compounds described herein containing a polymer. Polymers contemplated within the compounds described herein are preferably water soluble and substantially non-antigenic, and include, for example, polyalkylene oxides (PAO's). The compounds described herein further include linear, branched, or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

The polyalkylene oxide has the total number average molecular weight of from about 2,000 to about 100,000 daltons, preferably from about 5,000 to about 60,000 daltons. The polyalkylene oxide can be more preferably from about 5,000 to about 25,000 or from about 20,000 to about 45,000 daltons. In some particularly preferred embodiments, the compounds described herein include the polyalkylene oxide having the total number average molecular weight of from about 30,000 to about 45,000 daltons. In one particular embodiment, a polymeric portion has a total number average molecular weight of about 40,000 daltons.

In one preferred aspect, the compounds described herein include multi-arm polyethylene glycol polymers. The multi-arm polymers contemplated within the compounds described herein are water soluble and substantially non-antigenic.

The multi-arm PEGs have a total number average molecular weight of from about 2,000 to about 100,000 daltons, preferably from about 5,000 to about 60,000 daltons. The multi-arm PEGs can be more preferably from about 5,000 to about 25,000 or from about 20,000 to about 45,000 daltons. In some particularly preferred embodiments, the compounds described herein include multi-arm PEGs having a total number average molecular weight of from about 30,000 to about 45,000 daltons. In one particular embodiment, a polymeric portion has a total number average molecular weight of about 40,000 daltons.

PEG is generally represented by the structure:

—(CH₂CH₂O)ₙ—

where (n) is a positive integer of from about 10 to about 2300 so that the polymeric portion of the compounds described herein has a number average molecular weight of from about 2,000 to about 100,000 daltons. (n) represents the degree of polymerization for the polymer, and is dependent on the molecular weight of the polymer.

Alternatively, the each polymer arm can be represented by the structure:

-M₁-CH₂CH₂(OCH₂CH₂)ₙ—,

—(CH₂)_{d}-M₁-CH₂CH₂(OCH₂CH₂)ₙ— or

—C(═O)—(CH₂)_{d}-M₁-CH₂CH₂(OCH₂CH₂)ₙ—

wherein

M₁ is O, or S;

(d) is zero or a positive integer of from about 1 to about 10, preferably, 0, 1, 2, 3, and more preferably, zero or 1; and

(n) is a positive integer of from about 10 to about 2,300.

Suitable polymers as included in the compounds of Formula (I) correspond to polymer systems (IIIa)-(IIIh) with the following structure:

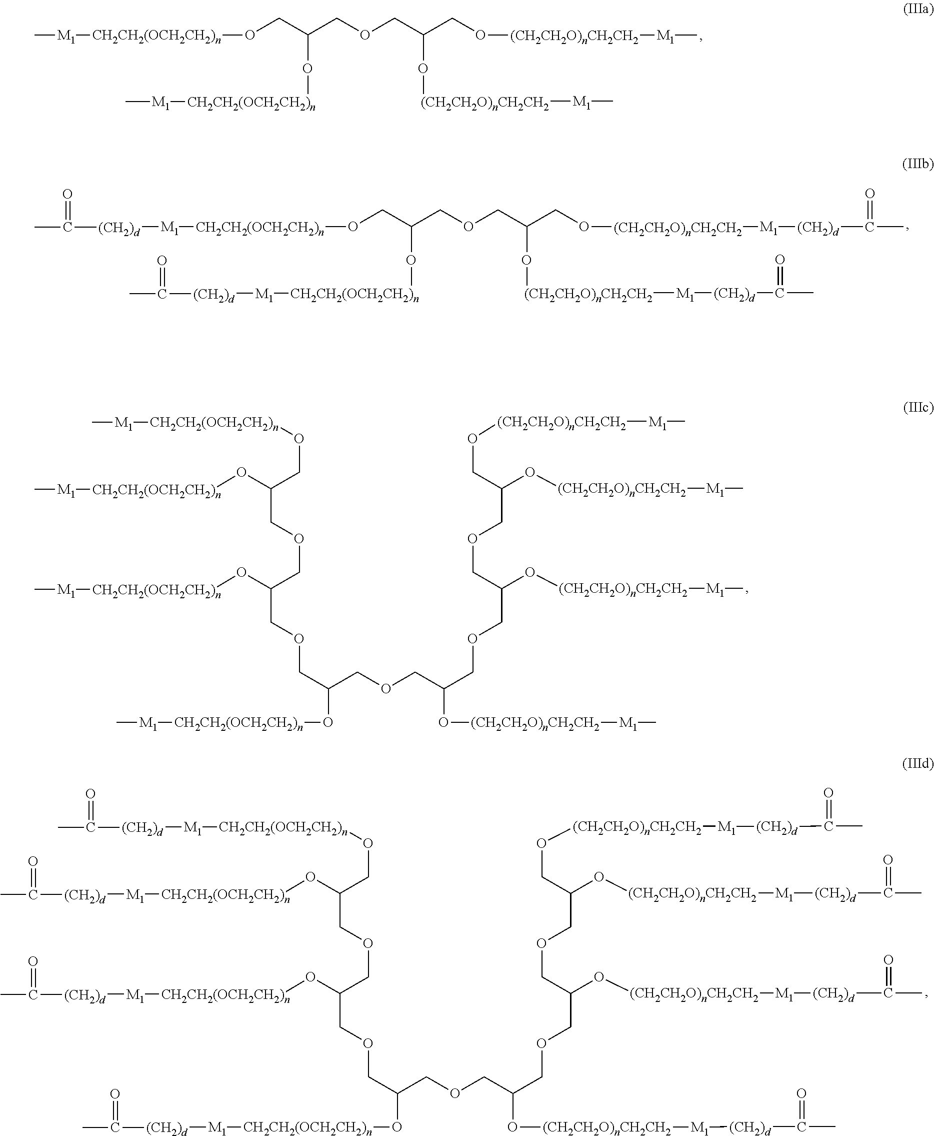

—(CH₂)_{d1}-M₁-CH₂CH₂(OCH₂CH₂)ₙOCH₂CH₂-M₁-(CH₂)_{d1}— (IIIe),

—C(═O)—(CH₂)_{d1}-M₁-CH₂CH₂(OCH₂CH₂)ₙOCH₂CH₂-M₁-(CH₂)_{d1}—C(═O)— (IIIf),

A-CH₂CH₂(OCH₂CH₂)ₙOCH₂CH₂-M₁-(CH₂)_{d1}— (IIIg), and

A-CH₂CH₂(OCH₂CH₂)ₙOCH₂CH₂-M₁-(CH₂)_{d1}—C(═O)— (IIIh),

wherein A is OH, C₁₋₆ alkoxy (e.g., methoxy, ethoxy), COOH, or amine; and

all other variables are as previously defined.

The multi-armed polymers prior to the conjugation to the compounds described herein include multi-arm PEG-OH products such as those described in NOF Corp. Drug Delivery System catalog, Ver. 8, April 2006, the disclosure of which is incorporated herein by reference. The polymers can be converted into suitably activated forms, using the activation techniques described in U.S. Pat. No. 5,122,614 or 5,808,096. Specifically, such a PEG can be of the formula:

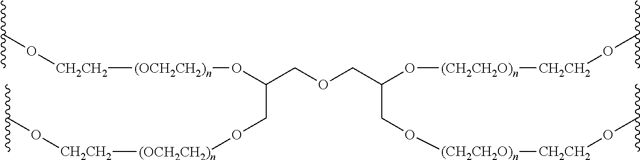

wherein:

(n) is an integer from about 4 to about 455.

In one embodiment, the degree of polymerization for the polymer (n) is from about 28 to about 341 to provide polymers having the total number average molecular weight of from about 5,000 Da to about 60,000 Da, and preferably from about 114 to about 239 to provide polymers having the total number average molecular weight of from about 20,000 Da to about 42,000 Da. (n) represents the number of repeating units in the polymer chain and is dependent on the molecular weight of the polymer. In one particular embodiment, (n) is about 227 to provide the polymeric portion having a total number average molecular weight of about 40,000 Da.

In certain embodiments, all four of the PEG arms can be converted to suitable activating groups, for facilitating attachment to other molecules (e.g., spacers and branching groups). Such compounds prior to conversion include:

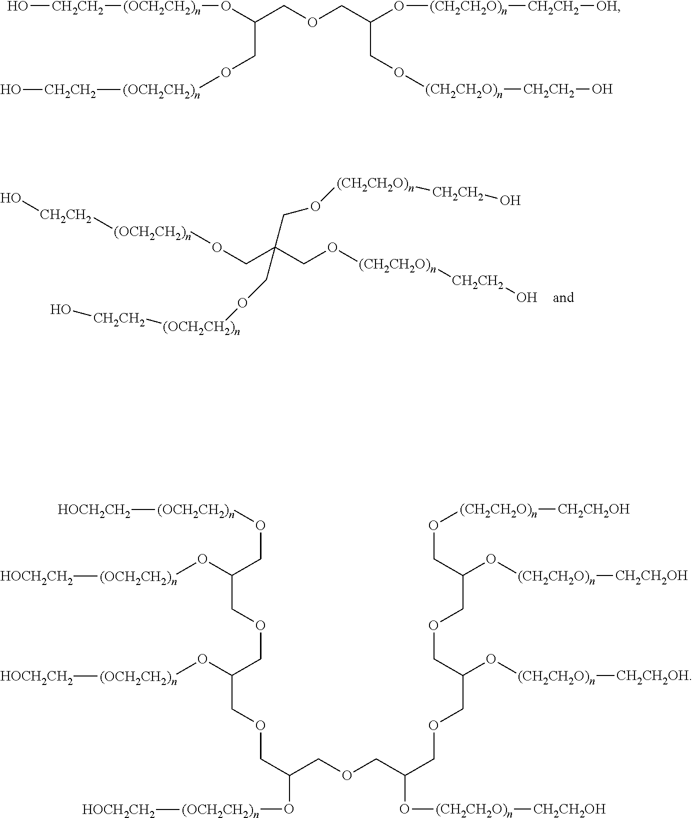

The multi-arm PEGs are conjugated to toyocamycin and analogs described herein via a spacer, and optionally with a branching group. The multi-arm polymers for conjugation to a compound of Formula (I) can be converted into suitably activated polymers, using the activation techniques described in U.S. Pat. Nos. 5,122,614 and 5,808,096 and other techniques known in the art without undue experimentation. For example, multi-arm PEGs can be activated using a similar technique as that used for activating linear PEGs except that a sufficient molar excess of activating agents is employed to ensure that all or substantially all of the terminal groups of each polymer arm are “activated.” Conjugation thereafter proceeds in the usual manner.

Examples of activated PEGs useful for the preparation of compounds of Formula (I) include, for example, a linear or multi-arm polyethylene glycol-succinimidyl carbonate (SC-PEG), a linear or multi-arm polyethylene glycol-succinimidyl succinate (SS-PEG), a linear or multi-arm polyethyleneglycol-carboxylic acid, a linear or multi-arm polyethylene glycol succinate and a linear or multi-arm polyethylene glycol-tresylate (PEG-TRES).

In some aspects, polymers having terminal carboxylic acid groups can be employed in the polymeric delivery systems described herein. Methods of preparing polymers having terminal carboxylic acids in high purity are described in U.S. Patent Application Publication No. 2007/0173615, the contents of which are incorporated herein by reference. The methods include first preparing a tertiary alkyl ester of a polyethylene glycol followed by conversion to the carboxylic acid derivative thereof. The first step of the preparation of linear or multi-arm PEG carboxylic acids includes forming an intermediate such as a t-butyl ester of a PEG. This intermediate is formed by reacting a PEG with a t-butyl haloacetate in the presence of a base such as potassium t-butoxide. Once the t-butyl ester intermediate has been formed, the carboxylic acid derivative of the PEG can be readily provided in high purity.

For purposes of the present invention, “substantially or effectively non-antigenic” means polymeric materials understood in the art as being nontoxic and not eliciting an appreciable immunogenic response in mammals.

### C. Leaving Groups and Activating Groups

In some aspects, suitable leaving/activating groups include, without limitations, halogen (F, Br, Cl, I), activated carbonate, carbonyl imidazole, cyclic imide thione, chloroformate, isocyanate, N-hydroxysuccinimidyl, para-nitrophenoxy (PNP), N-hydroxyphthalamide, N-hydroxybenzotriazolyl (N-HOBT), tosylate, mesylate, tresylate, nosylate, C₁-C₆ alkyloxy, C₁-C₆ alkanoyloxy, arylcarbonyloxy, ortho-nitrophenoxy, imidazole, pentafluorophenoxy, 1,3,5-trichlorophenoxy, and 1,3,5-trifluorophenoxy or other suitable leaving groups, as will be apparent to those of ordinary skill. In one preferred embodiment, the leaving/activating groups can be N-hydroxysuccinimidyl, N-hydroxybenzotriazolyl (N—HOBT), cyclic imide thione, or para-nitrophenoxy (PNP).

For purposes of the present invention, leaving/activating groups are to be understood as those groups which are capable of reacting with a nucleophile found on spacers, branching groups, toyocamycin or analogs, multi-arm polymers, toyocamycin-spacer intermediates, etc. The nucleophile thus contains a group for displacement, such as OH, NH₂ or SH group.

### D. Synthesis of Compounds of Formula (I)

Generally, the compounds of the present invention are prepared by reacting one or more equivalents of an activated linear or multi-arm polymer with, for example, one or more equivalents of an adenine nucloeside analog (e.g., toyocamycin) per polymer arm terminal under conditions which are sufficient to effectively cause the adenine nucleoside analog to undergo a reaction with the activated polymer to form a polymer conjugate of adenine nucleoside analog via a spacer.

More specifically, the methods can include:

1) providing one equivalent of an adenine nucleoside analog (e.g., toyocamycin or its analog) containing an available amino group and one or more equivalents of a bifunctional spacer containing an available carboxylic acid group;

2) reacting the two reactants to form an adenine nucleoside analog-spacer amide intermediate in an inert solvent such as DCM (or DMF, chloroform, toluene or mixtures thereof) in the presence of a coupling reagent such as 1-(3-dimethyl aminopropyl)3-ethyl carbodiimide (EDC), 1,3-diisopropylcarbodiimide (DIPC) or suitable dialkyl carbodiimide, Mukaiyama reagents (2-halo-1-alkyl-pyridinium halides) or propane phosphonic acid cyclic anhydride (PPACA), etc, and a suitable base such as DMAP, or reacting an activated bifunctional spacer with an adenine nucleoside analog with a suitable base in an inert solvent such as DCM (or DMF, chloroform, toluene or mixtures thereof); and

3) reacting one or more equivalents per polymer arm terminal (2 eq. in Example) of the resulting intermediate having an amine group and one equivalent of an activated polymer, such as a four-arm PEG-succinimidyl carbonate in an inert solvent such as DCM (or DMF, chloroform, toluene or mixtures thereof) in the presence of a base, or one equivalent of a four-arm PEG-carboxylic acid in the presence of a coupling reagent such as 1-(3-dimethyl aminopropyl) 3-ethyl carbodiimide (EDC), 1,3-diisopropylcarbodiimide (DIPC) or suitable dialkyl carbodiimide, Mukaiyama reagents (2-halo-1-alkyl-pyridinium halides) or propane phosphonic acid cyclic anhydride (PPACA), etc, and a suitable base such as DMAP; which are available, for example, from commercial sources such as Sigma Chemical, or synthesized using known techniques, at a temperature from 0° C. up to 22° C.

In one preferred aspect, the hydroxyl group of adenine nucleoside analog (e.g., toyocamycin or its analog) is protected prior to step 1) and the protecting group is removed after step 3). Useful hydroxyl protecting groups include acetyl, TBDMS, TMS, TES, allyl, or other known suitable hydroxyl protecting groups.

The activated multi-arm polymers (e.g., a polymer containing 1-4 terminal carboxyl acid groups) can be prepared, for example, by converting NOF Sunbright-type or other branched multi-arm polymers having terminal OH groups into the corresponding carboxyl acid derivatives using standard techniques well known to those of ordinary skill. See, for example, commonly assigned U.S. Pat. No. 5,605,976, and U.S. Patent Publication No. 2007/0173615, the contents of which are incorporated herein by reference.

The coupling agents in steps 2) and 3) can be the same or different.

In one embodiment, the compounds described herein are prepared by the steps including:

(a) reacting one equivalent of an adenine nucleoside (e.g., toyocamycin) with one or more equivalents of a bifunctional spacer containing an available carboxylic acid group or activated carboxylic acid group under conditions effective to form an adenine nucleoside-spacer amide intermediate having an available amine group; and

(b) reacting one of more equivalents of the resulting intermediate from step (a) per polymer arm terminal with one equivalent of an activated polymer,

under conditions effective to form a compound of Formula (I),

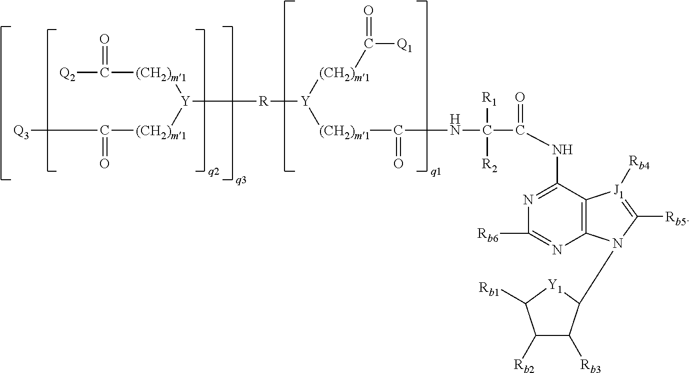

Examples of preferred bifunctional spacer linker groups include leucine, 2-aminoisobutyric acid, etc. and syntheses of polymeric conjugates of adenine nucleoside analogs are shown in the Examples. Alternative and specific syntheses are provided in the examples.

Examples of compounds prepared according to the present invention include, but are not limited to:

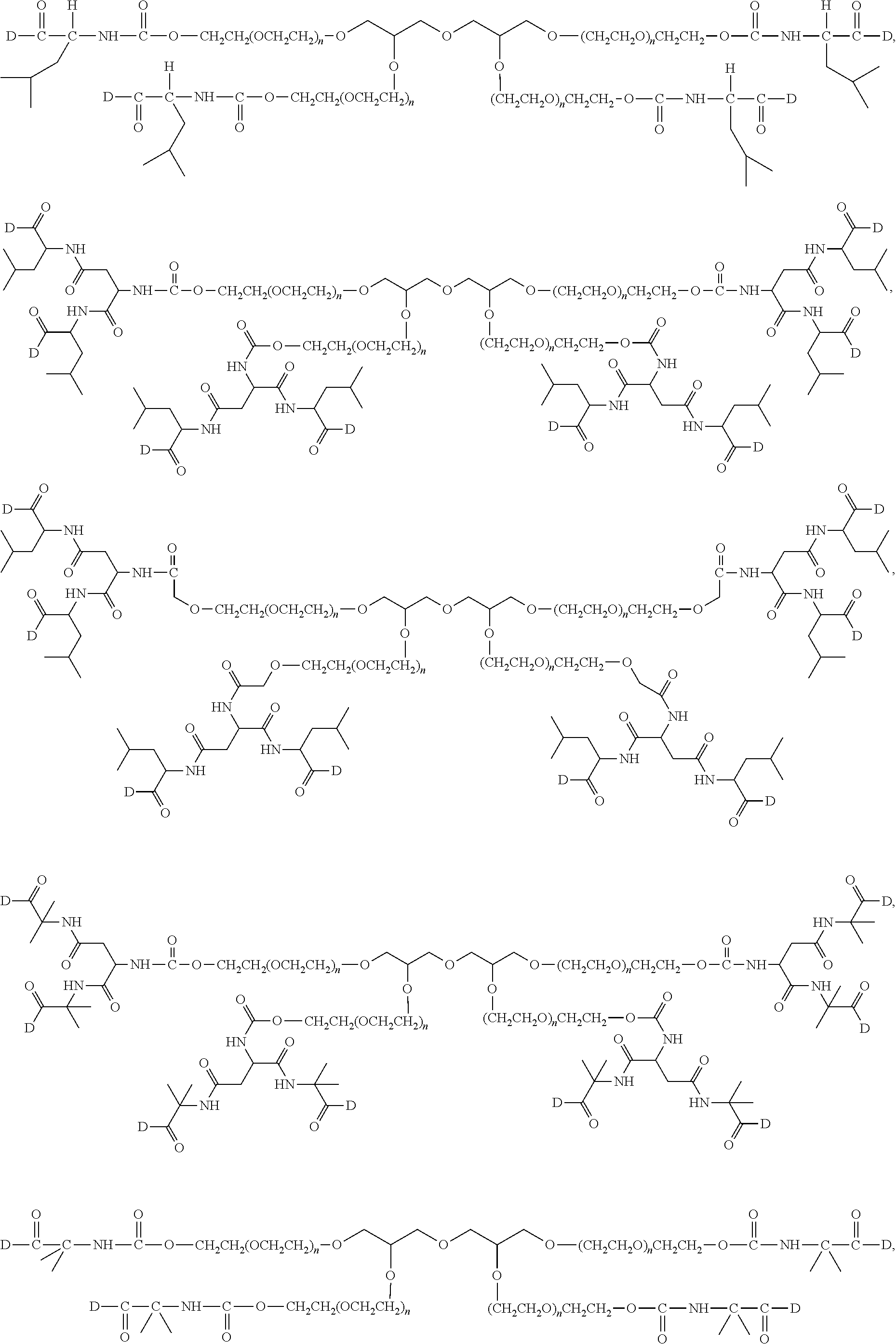

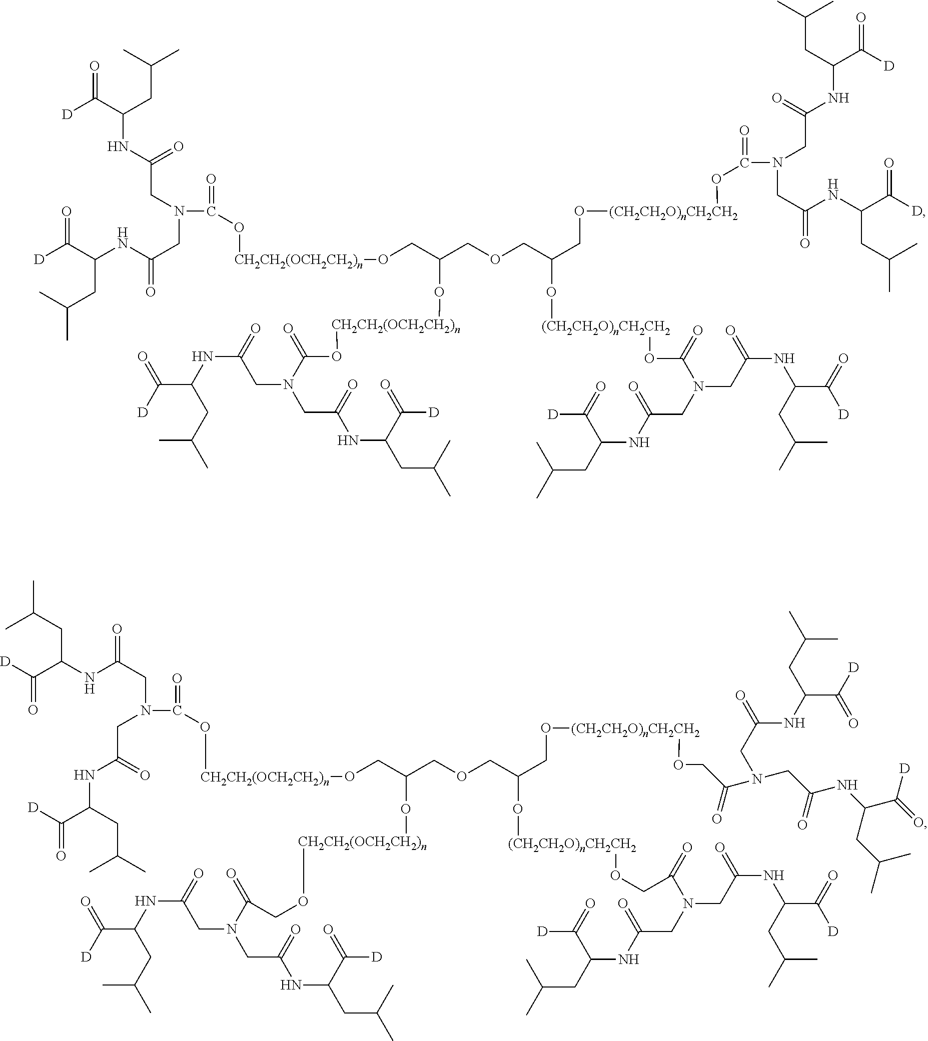

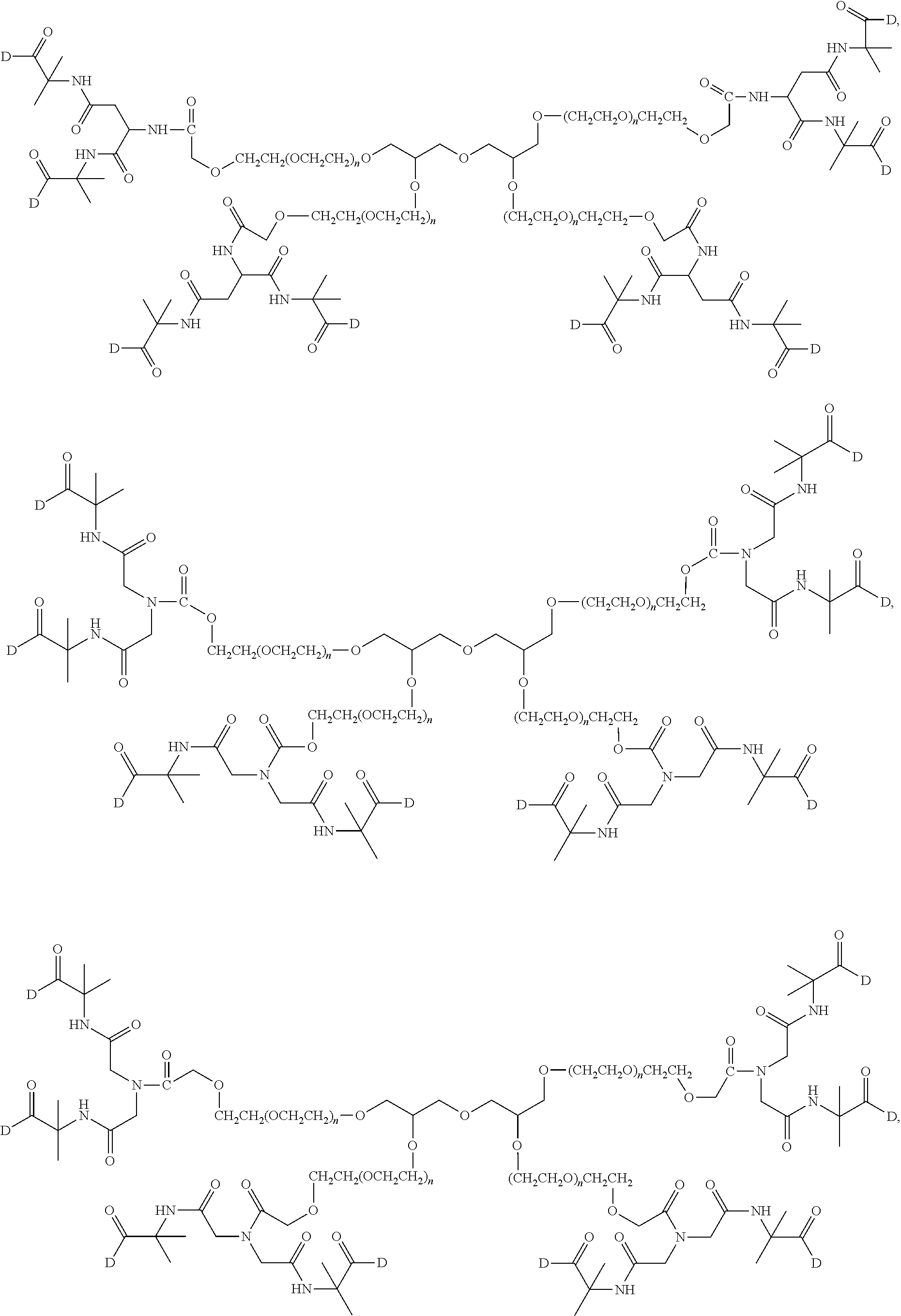

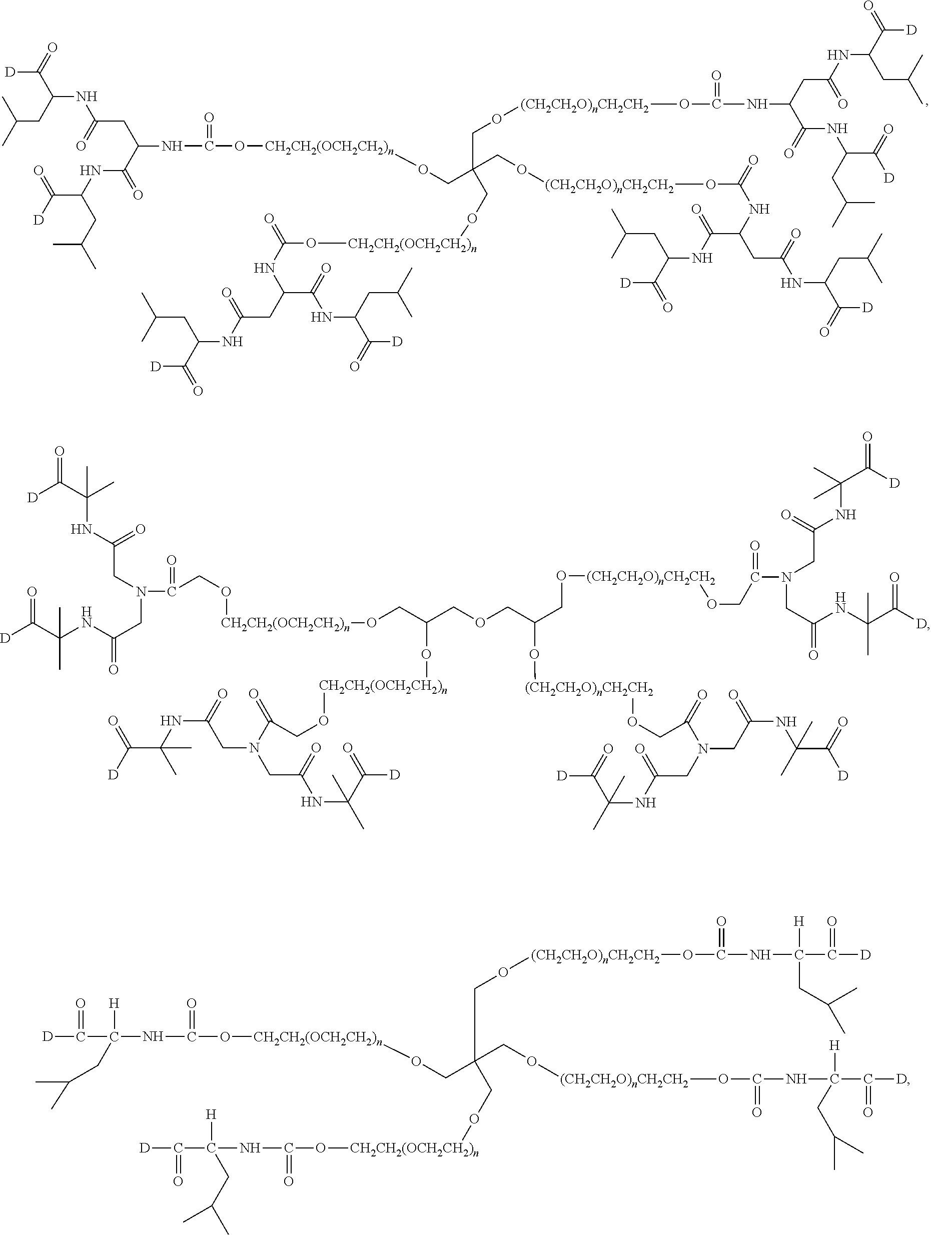

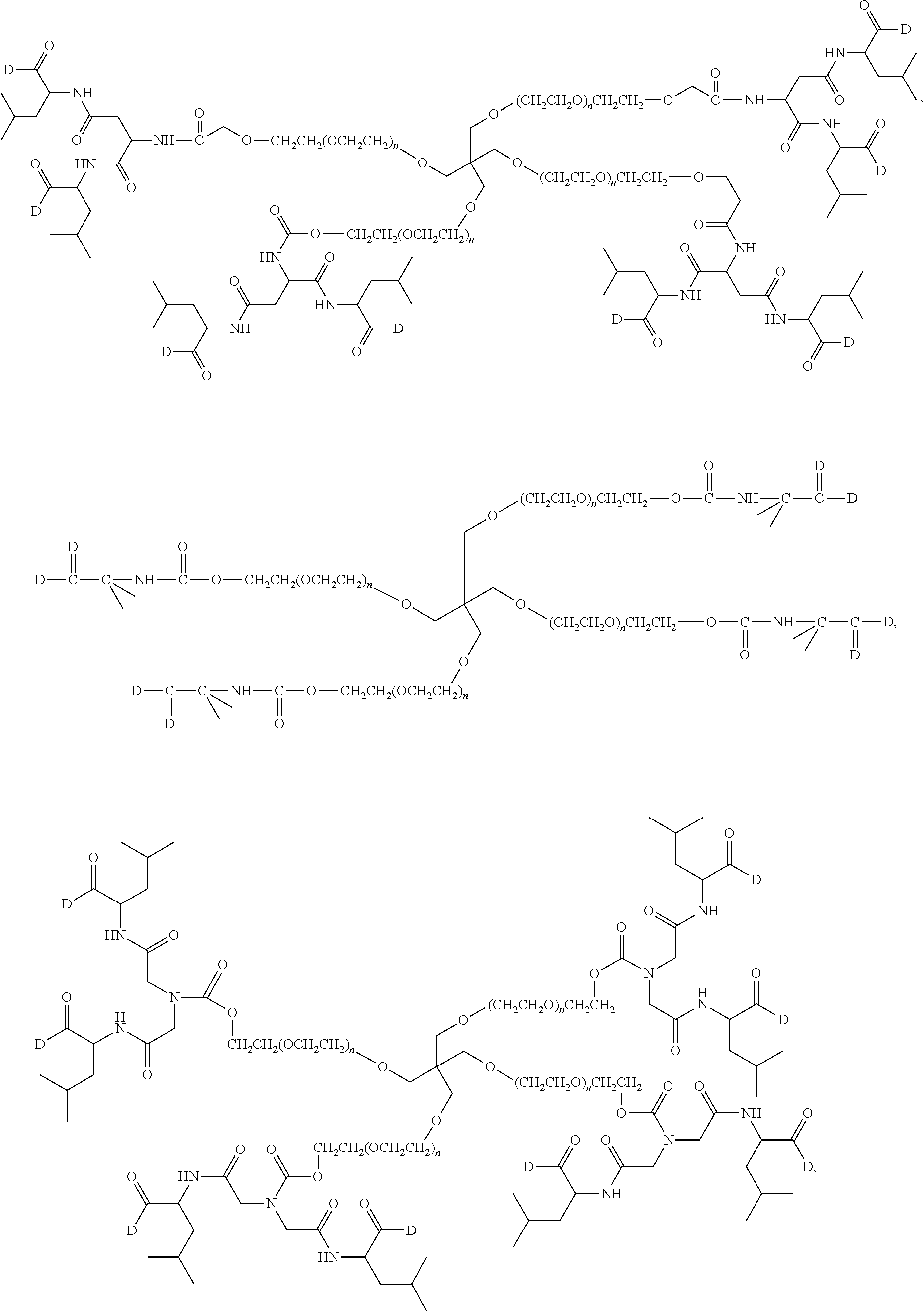

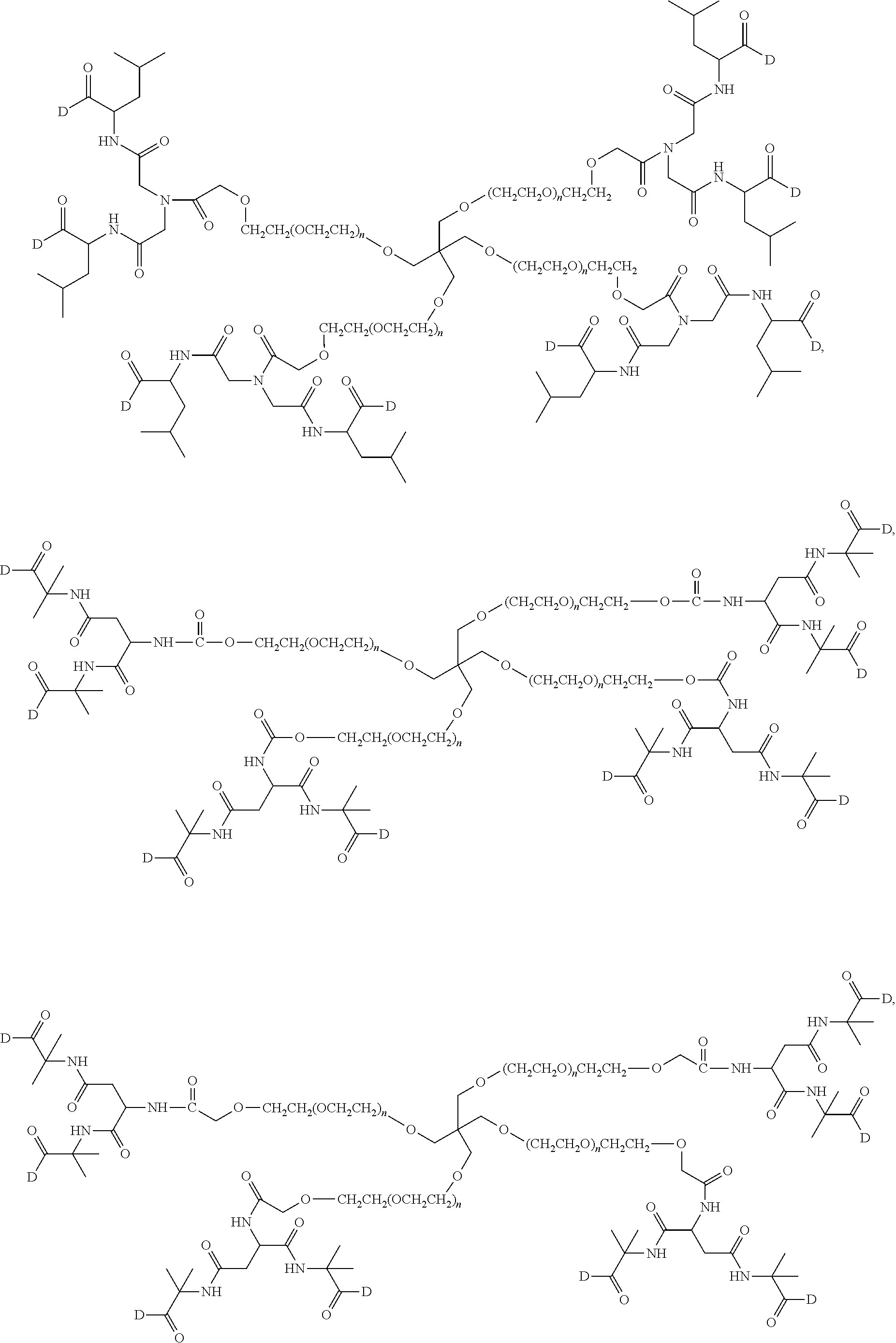

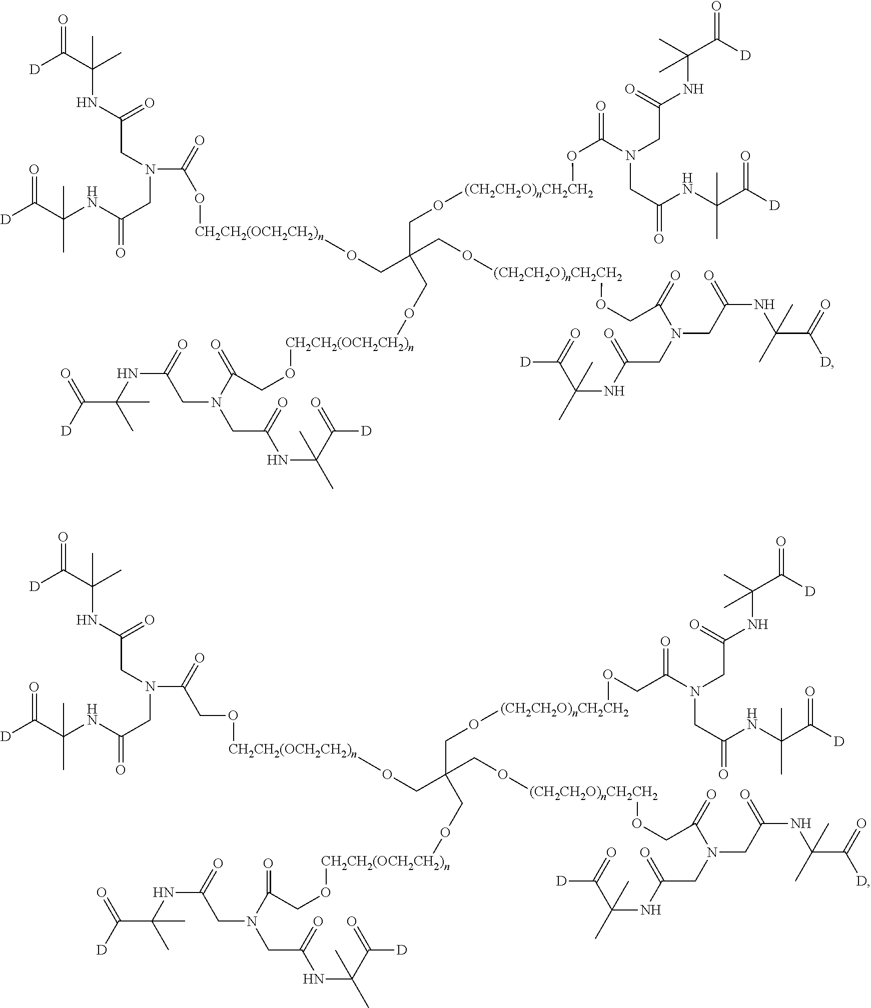

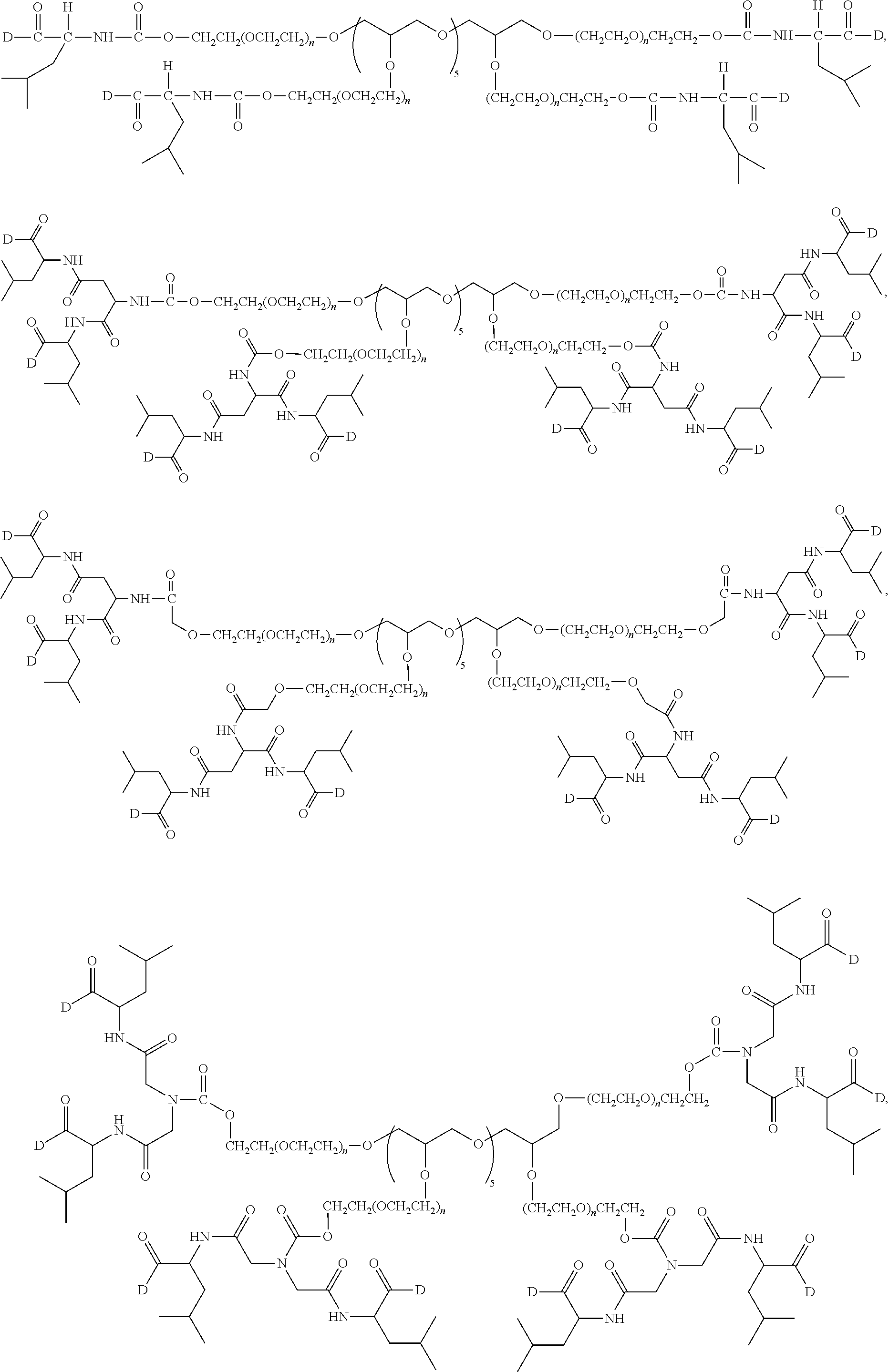

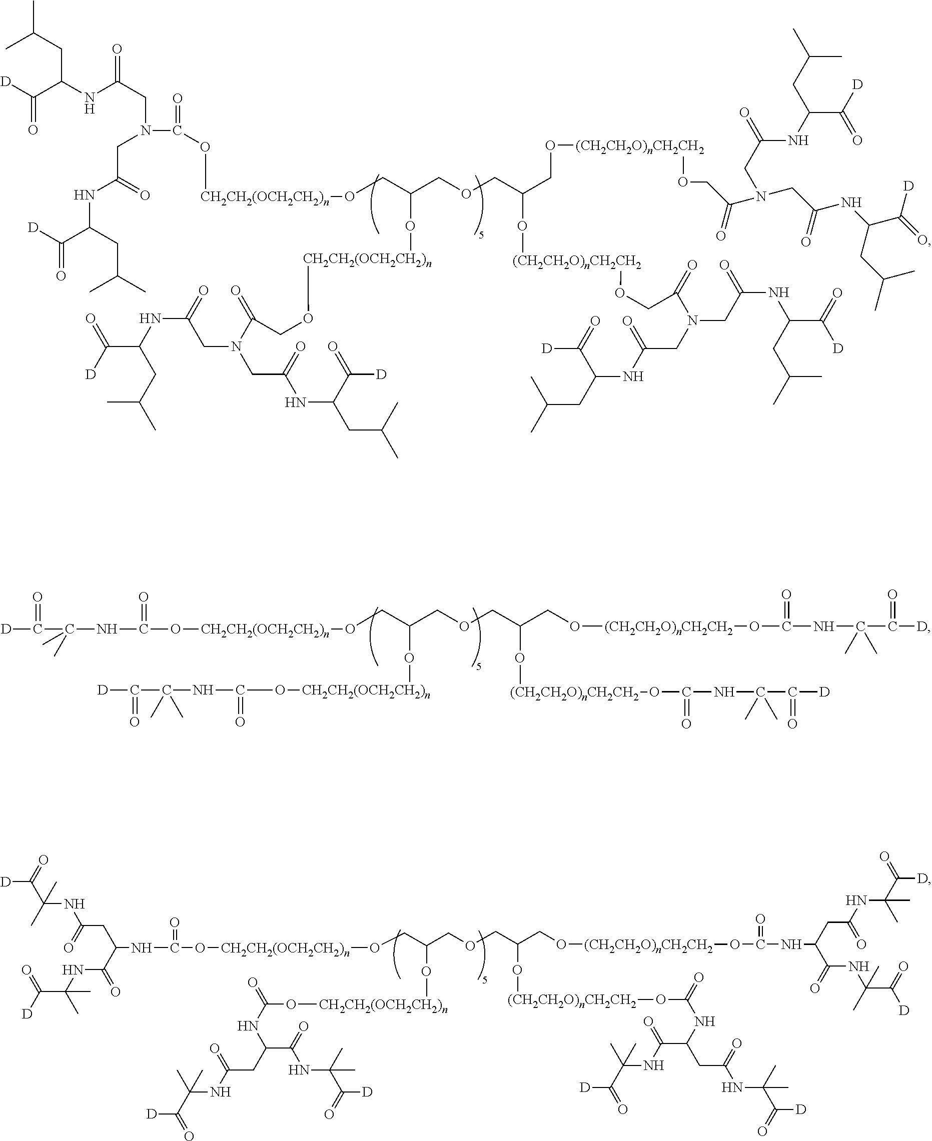

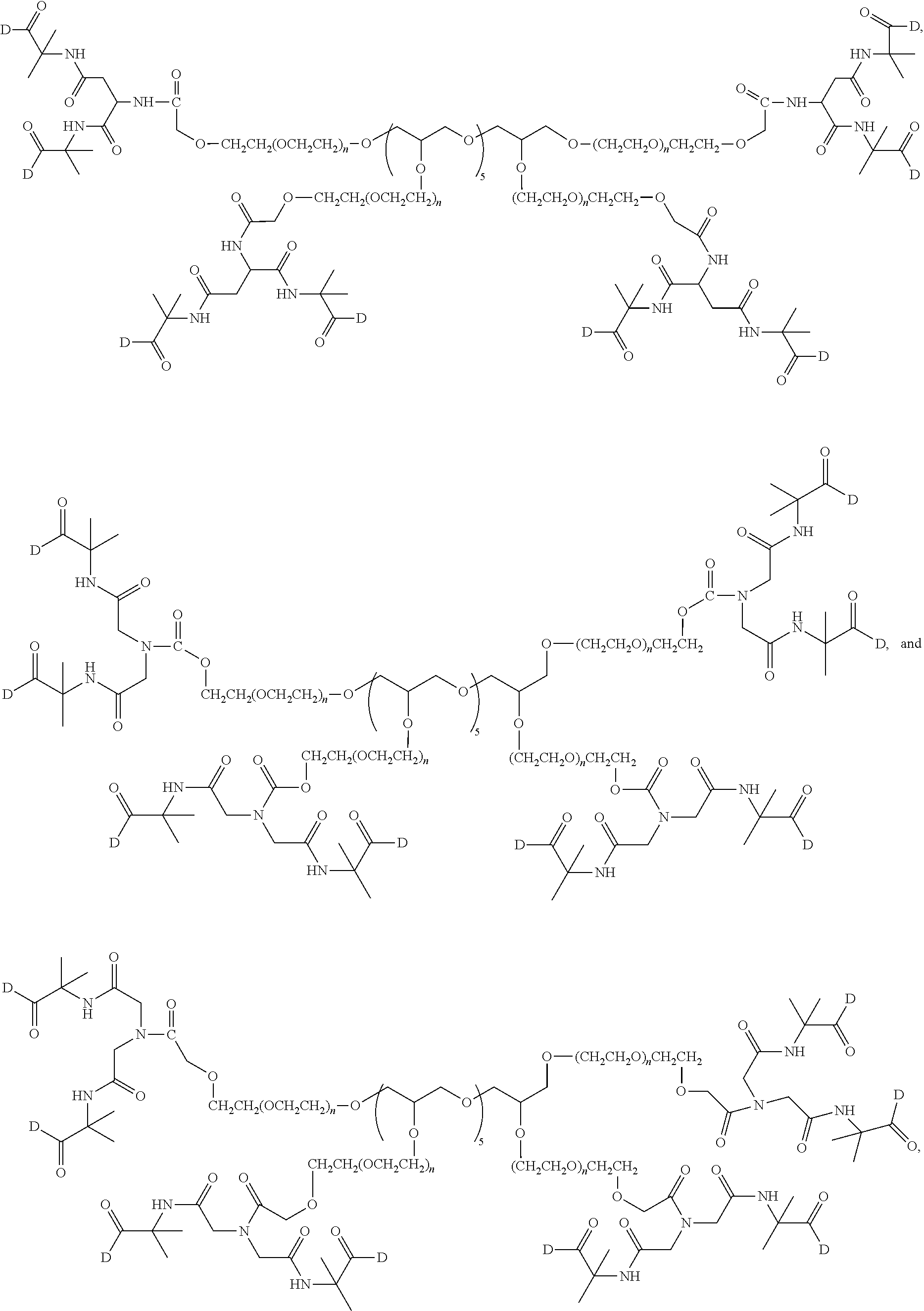

wherein D is

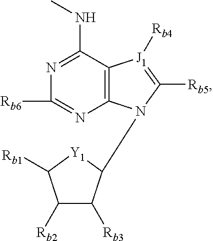

and

all other variables are as previously defined.

In one preferred embodiment, D is 2′-deoxyadenosine, toyocamycine, sangivamycin, ARC, fludarabine, cladribine, clofarabine, 6-aminotoyocamycin, tubercidin, and mono-, di-, or triphosphate thereof, and (n) is about 227, so that the total average molecular weight of the polymeric portion of the compound is about 40,000 daltons.

### E. Compositions/Formulations

Pharmaceutical compositions containing the compounds of the present invention may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention.

For injection, including, without limitation, intravenous, intramuscular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds described herein may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the compounds described herein with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

### F. Use of Compounds of Formula (I)

In one aspect of the present invention, the compounds of the present invention can be useful in therapy associated with adenine nucleoside analogs (e.g., toyocamycin and analogs) in mammals. The methods include administering or delivering the compounds described herein to a mammal in need thereof. The methods can include

(a) forming a polymeric conjugate of adenine nucleoside analog (e.g., toyocamycin or an analog thereof); and

(b) administering an effective amount of a conjugate represented by Formula (I) to a mammal in need thereof.

In one embodiment, there are provided methods of treating a patient having a malignant tumor or cancer, comprising administering an effective amount of a pharmaceutical composition containing the compounds described herein to a patient in need thereof. The cancer being treated can be one or more of the following: solid tumors, lymphomas, small cell lung cancer, acute myeloid leukemia (AML), acute lymphocytic leukemia (ALL), renal cancer, breast cancer, pancreatic cancer, glioblastoma, ovarian cancer, gastric cancers, colorectal cancer, prostate cancer, cervical cancer, brain tumors, KB cancer, lung cancer, colon cancer, epidermal cancer, melanoma, etc. The compounds of the present invention are useful for treating neoplastic disease, reducing tumor burden, reducing metastasis of neoplasms and reducing recurrences of tumor/neoplastic growths in mammals. In one example, the treatment is conducted in which the compounds described herein provide toyocamycin. In another example, compounds of Formula (I) containing tubercidin are administered to mammals in the treatment of leukemia, sarcoma, adenocarcinoma, mammary carcinoma, etc. In another example, compounds described herein containing 6-aminotoyocamycin are administered to mammals for treating renal cancer.

In this aspect, “treatment” shall be understood to mean inhibition, reduction, and amelioration of tumor growth, tumor burden and metastasis, remission of tumor, or reduction of recurrences of tumor and/or neoplastic growths in patients after completion of treatment.

In another embodiment, the present invention provides a method of inhibiting the growth or proliferation of cancer cells in a mammal. The method includes administering a compound described herein to a mammal having cancer.

Treatment is deemed to occur when a patient achieves positive clinical results. For example, successful treatment shall be deemed to occur when at least 20% or preferably 30%, more preferably 40% or higher (i.e., 50%) decrease in tumor growth including other clinical markers contemplated by the artisan in the field is realized when compared to that observed in the absence of the treatment described herein. Other methods for determining changes in a tumor clinical status resulting from the treatment described herein include: biopsies such as tumor biopsy; immunohistochemistry study using antibody, radioisotope, dye; and complete blood count (CBC).

In certain aspects, clinical response criteria defined according to RECIST guidelines can be useful. Complete response (CR) is defined as complete disappearance of measurable and evaluable clinical evidence of cancer. Partial response (PR) is defined as at least a 50% reduction in the size of all measurable tumor areas. Progressive disease (PD) is defined as an increase of >25% (compared to baseline or best response) in the size of all measurable tumor areas. Stable disease (SD) is defined as neither sufficient shrinkage to qualify for PR nor sufficient increase to qualify for PD. Treatment is deemed to occur, when CR, PR and/or SD are achieved.

Yet another embodiment according to the present invention provides methods of modulating/inhibiting angiogenesis or angiogenic activity in a mammal. The angiogenesis is a tumoral angiogenesis or tumor-dependent angiogenesis. Useful systems for determining changes in angiogenesis include chicken chorioallantoic membrane (CAM) assay. Other systems includes bovine capillary endothelial (BCE) cell assay (e.g., U.S. Pat. No. 6,024,688), and HUVEC (human umbilical cord vascular endothelial cell) growth inhibition assay (e.g., U.S. Pat. No. 6,060,449).

In yet another embodiment, the present invention provides a method of treating a viral infection (e.g. hepatitis C infections) in a mammal. The method includes administering an effective amount of a compound described herein to a mammal in need thereof.

In yet a further embodiment, the methods described herein can be useful in the treatment of patients with diseases associated with abnormally high levels of VEGF expression, as compared to normal subjects. Levels of VEGF expression can be measured by techniques known in the art, including the measurement of VEGF mRNA expression.

Yet another and/or further embodiment according to the present invention provides methods of enhancing the therapeutic effects of toyocamycin in a mammal. The method includes administering an effective amount of the compound described herein, wherein the T_{1/2} of released toyocamycin in blood ranges within about 10 to about 300% of 10 minutes, preferably about 10% to about 80% of 10 minutes. The method is conducted by administering compounds described herein in which R₁ is hydrogen and R₂ is isobutyl. Alternatively, the T_{1/2} of released toyocamycin in blood ranges within about 50 to about 150% of 4 hours, preferably about 80% to about 100% of 4 hours. The method employs compounds described herein in which R₁ and R₂ are methyl.

In many aspects of the present invention, the methods employ use of compounds of Formula (I) or pharmaceutical salt thereof to a mammal in need thereof, wherein D is toyocamycin or sangivamycin. In one example, the methods are conducted in which the compounds described herein have the structure:

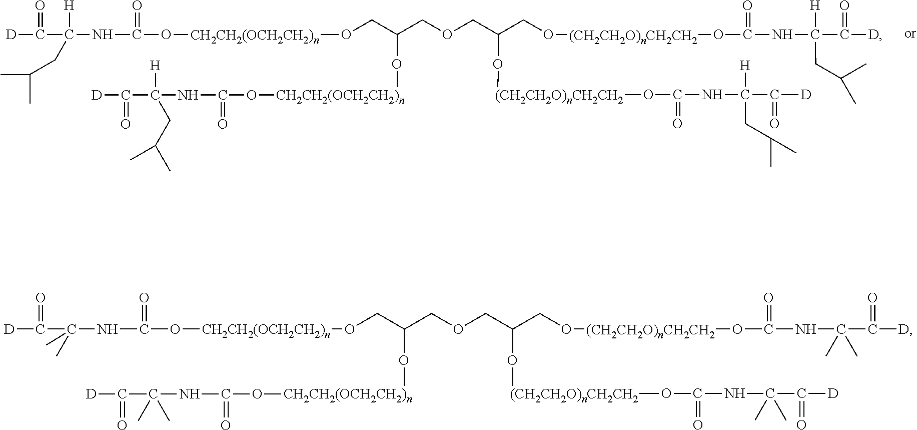

wherein D is

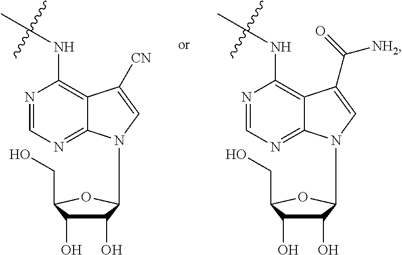

and
(n) is about 227, so that the total average number molecular weight of the polymeric portion of the compound is about 40,000 daltons.

Preferably, the administering step includes administration via the blood stream of the mammal (i.v.).

A therapeutically effective amount means an amount of compound effective to reduce, alleviate or ameliorate symptoms of disease or prolong the survival of the subject being treated with the compounds described herein. For compounds used in the methods described herein, the therapeutically effective amount can be estimated initially from in vitro assays. Then, the dosage can be formulated for use in animal models so as to achieve a circulating concentration range that includes the effective dosage. Such information can be used to more accurately determine dosages useful in patients.

The amount of the composition, e.g., used as a prodrug, that is administered will depend upon the parent molecule included therein. Generally, the amount of prodrug used in the treatment methods is that amount which effectively achieves the desired therapeutic result in mammals. Naturally, the dosages of the various prodrug compounds can vary somewhat depending upon the parent compound, rate of in vivo hydrolysis, molecular weight of the polymer, etc. In addition, the dosage, of course, can vary depending upon the dosage form and route of administration.

In general, adenine nucleoside analogs (e.g., toyocamycin and analogs) are administered to mammals in amounts ranging from about 0.1 to about 5 mg/kg/dose. For example, toyocamycin can be given at about 1 or 5 mg/kg/dose. In one embodiment, toyocamycin and analogs can be administered to a patient in amounts of from about 10 to about 200 μg/kg/dose (e.g., from about 10-100 μg/kg/dose, from about 10-80 μg/kg/dose, from about 70-150 μg/kg/dose).

The treatment protocol can be based on a single dose treatment protocol or divided into multiple doses which are given as part of a multi-week treatment protocol. It is also contemplated that the treatment will be given for one or more cycles until the desired clinical result is obtained. The exact amount, frequency and period of administration of the compound of the present invention will vary, of course, depending upon the sex, age and medical condition of the patient as well as the severity of the disease as determined by the attending clinician.

In all aspects of the invention where polymeric conjugates described herein are administered, the dosage amount mentioned is based on the amount of adenine nucleoside analogs (e.g., toyocamycin and analogs) rather than the amount of polymeric conjugate administered. The actual weight of the PEG-conjugated adenine nucleoside analog (e.g., toyocamycin) will vary depending on the weight of the linear or multi-arm PEG and the loading of the active agent per multi-arm PEG (e.g., up to four equivalents of adenine nucleoside analog (e.g., toyocamycin) per four-arm PEG, up to eight equivalents of adenine nucleoside analog (e.g., toyocamycin) per branched four-arm PEG).

The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. The precise dose will depend on the stage and severity of the condition, and the individual characteristics of the patient being treated, as will be appreciated by one of ordinary skill in the art.

Additionally, toxicity and therapeutic efficacy of the compounds described herein can be determined by standard pharmaceutical procedures in cell cultures or experimental animals using methods well-known in the art.

Further aspects of the present invention include combining the compounds described herein with other anticancer therapies (e.g., radiotherapy or chemotherapies employing other chemotherapeutic agents) for synergistic or additive benefit. Thus, the compounds described herein can be administered prior to, during, or after other anticancer therapy. One embodiment includes concurrent administration of compounds described herein and radiotherapy in cancer treatment.

### EXAMPLES

The following examples serve to provide further appreciation of the invention but are not meant in any way to restrict the effective scope of the invention. The following examples serve to provide further appreciation of the invention but are not meant in any way to restrict the effective scope of the invention. The underlined and bold-faced numbers recited in the Examples correspond to those shown in the Figures.

### General.

All reactions were run under an atmosphere of dry nitrogen or argon. Commercial reagents were used without further purification. All PEG compounds were dried under vacuum or by azeotropic distillation (toluene) prior to use.

### Abbreviations.

DCM (dichloromethane), DIEA (N,N-diisopropylethylamine), DMAP (4-(dimethylamino)pyridine), DMF (N,N-dimethylformamide), DSC (N,N′-disuccinimidyl carbonate), EDC (1-ethyl-3-(3-dimethylaminopropyl)carbodiimide), IPA (2-propanol), HOBT (1-hydroxybenzotriazole), NMM (N-methylmorpholine), TBDMS-Cl (tert-butyl dimethyl silyl chloride), TFA (trifluoroacetic acid), TEAA (tetraethylammonium acetate).

### Example 1

### General NMR Method

¹H spectra were obtained with a Varian MercuryVX-300 instrument using deuteriochloroform as solvent unless specified. ¹³C NMR spectra were obtained at 75.46 MHz on the Varian MercuryVX-300. Chemical shifts (δ) are reported in parts per million (ppm) downfield from tetramethylsilane (TMS) and coupling constants (J values) are given in hertz (Hz).

### Example 2

### HPLC Method

Analytical HPLC's were performed using a size exclusion column (PolySep-GFC-P3000, Phenomenex) under isocratic conditions with a 1:1 mixture (v/v) of methanol-water as mobile phase. Peak elution was monitored at 275 nm using a UV detector. To detect the presence of any free PEG and to confirm the presence of PEGylated conjugates, an evaporative light scattering detector (ELSD), Model 5000 ELSD (Alltech) was employed. Based on ELSD and UV analysis, all the final PEGylated products were free of native drug and were ≧95% pure by HPLC.

### Example 3

### Analysis of Toyocamycin and Toyocamycin Content in PEG Conjugates

Test sample solutions (1 mg/mL) and standard toyocamycin solutions in four to five different concentration ranging from 10 μg/mL to 100 μg/mL in water were treated with 50 mM Na₂CO₃ at pH 10.8 for two hours at room temperature. The amount of toyocamycin in the resulting solutions were analyzed by measuring UV absorbance at 275 nm using RP HPLC with Aqua C18, 150×4.6 mm 300 Å column and the amount of toyocamycin was calculated against the standard solution.

### Example 4

### Determination of Hydrolysis Rates of PEG Conjugates

The rates of hydrolysis were obtained by employing a C18 reversed phase column (Jupiter®) using a gradient mobile phase consisting of (a) 0.05 M TFA buffer and (b) acetonitrile. A flow rate of 1 mL/min was used, and chromatograms were monitored using a UV detector at 260 nm for toyocamycin. For hydrolysis in plasma, the derivatives were dissolved in acetonitrile/MeOH at a concentration of 20 mg/mL. The solution was divided into vials with 100 μL and the solvent removed in vacuo. To the residue, 100 μL of plasma was added, then vortexed for 10 sec. The solutions were incubated at 37° C. for various periods of time. A mixture of methanol-acetonitrile (1:1, v/v, 400 μL) was added to a vial at the proper interval and the mixture was vortexed for 1 min, followed by filtration through 0.2 mm filter membrane. An aliquot of 40 μL of the filtrate was injected into the HPLC. On the basis of the peak area, the amounts of native compound and PEG conjugates were estimated, and the half-life of each compound in different media was calculated using linear regression analysis from the disappearance of PEG derivative.

### Example 5

### Preparation of Boc-Leu-Toyocamycin (Compound 3)

Anhydrous DCM (3 mL) was added to a solution of toyocamycin (1, 0.742 mmol) in anhydrous DMF (3 mL) at 0° C., followed by addition of Boc-Leu-OSu (2, 0.742 mmol) and DMAP (0.742 mmol). The reaction mixture was allowed to warm to room temperature with stirring overnight. The mixture was concentrated in vacuo and the residue was purified by prep HPLC using C18 column to give the product. ¹H and ¹³C NMR confirmed the structures.

### Example 6

### Preparation of TFA Leu-Toyocamycin (Compound 4)

A mixture of Boc-Leu-Toyocamycin (3, 0.2 mmol) and anhydrous DCM-TFA (1 mL/1 mL) was stirred at 0° C. for 30 minutes and the reaction progress was monitored by TLC. Upon completion of the reaction, the solvent was removed in vacuo and the residue was washed with anhydrous ether several times and dried in vacuo. HPLC confirmed the completion of reaction and the crude product was used for the next step without further purification.

### Example 7

### Preparation of 40 k 4-Arm PEG-carbamate-Leu-Toyocamycin (Compound 6)

A mixture of 40 k 4-arm SC-PEG (5, 800 mg, 0.02 mmol), Leu-Toyocamycin TFA salt (4, 0.2 mmol), DIEA (0.2 mmol), and DMAP (0.04 mmol) in a mixture of anhydrous DCM-DMF (6 mL/1 mL) was stirred at 0° C. to room temperature overnight. The reaction mixture was concentrated in vacuo and the residue was recrystallized from DMF/IPA twice to give 700 mg of product. ¹³C NMR confirmed that the structure of the product. The content of toyocamycin measured by UV was about 2.3-2.9% wt/wt and the purity measure by HPLC was 100%.

### Example 8

### Preparation of Boc-Aib-Toyocamycin (Compound 8)

A mixture of toyocamycin (1, 0.963 mmol), Boc-Aib-OH (7, 0.963 mmol), EDC HCl (1.059 mmol), and DMAP (1.44 mmol) in anhydrous DMF-DCM (3 mL/3 mL) was stirred at 0° C. to room temperature overnight. The mixture was concentrated in vacuo and the residue was purified by prep HPLC using C18 column to give the product. ¹H and ¹³C NMR confirmed the structures.

### Example 9

### Preparation of TFA Aib-Toyocamycin (Compound 9)

A mixture of Boc-Aib-Toyocamycin (8, 0.26 mmol) and anhydrous DCM-TFA (1 mL/1 mL) was stirred at 0° C. for 30 minutes and the reaction progress was monitored by TLC. Upon completion of the reaction, the solvent was removed in vacuo and the residue was washed with anhydrous ether several times and dried in vacuo. HPLC confirmed the completion of reaction and the crude product was used for the next step without further purification.

### Example 10

### Preparation of 40 k 4-Arm PEG-Carbamate-Aib-Toyocamycin (Compound 10)

A mixture of 40 k 4-arm SC-PEG (5, 1.47 g, 0.037 mmol), Aib-Toyocamycin TFA salt (9, 0.26 mmol), DIEA (0.514 mmol), and DMAP (0.0735 mmol) in a mixture of anhydrous DCM (15 mL) was stirred at 0° C. to room temperature overnight. The reaction mixture was concentrated in vacuo and the residue was recrystallized from DMF/IPA twice to give the product. ¹³C NMR confirmed that the structure of the product. The content of toyocamycin measured by UV was about 2.3-2.9% wt/wt and the purity measure by HPLC was 96.7%.

### Example 11

### Preparation of Boc-Val-Toyocamycin (Compound 12)

A mixture of toyocamycin (1. 0.963 mmol), Boc-Val-OH (11, 0.963 mmol), EDC HCl (1.059 mmol), and DMAP (1.44 mmol) in anhydrous DMF-DCM (3 mL/3 mL) is stirred at 0° C. to room temperature overnight. The mixture is concentrated in vacuo and the residue is purified by prep HPLC using C18 column to give the product.

### Example 12

### Preparation of TFA Val-Toyocamycin (Compound 13)

A mixture of Boc-Val-Toyocamycin (12, 0.26 mmol) and anhydrous DCM-TFA (1 mL/1 mL) is stirred at 0° C. for 30 minutes and the reaction progress is monitored by TLC. Upon completion of the reaction, the solvent is removed in vacuo and the residue is washed with anhydrous ether several times and dried in vacuo. Completion of reaction is confirmed by HPLC and the crude product is used for the next step without further purification.

### Example 13

### Preparation of 40 k 4-Arm PEG-Carbamate-Val-Toyocamycin (Compound 14)

A mixture of 40 k 4-arm SC-PEG (5, 1.47 g, 0.037 mmol), Val-Toyocamycin TFA salt (13, 0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 14

### Preparation of 40 k 4-Arm PEG-Asp(OMe)₂ (Compound 17)

A mixture of 40 k 4-arm PEG acid (15, 0.037 mmol), L-aspartic acid dimethylester.HCl (16, 0.26 mmol), EDC.HCl (0.52 mmol), and DMAP (0.52 mmol) in anhydrous DCM (15 mL) is stirred at room temperature overnight. The solvent is removed and the residue crystallized from 2-propanol to give the product.

### Example 15

### Preparation of 40 k 4-Arm PEG-Asp(OH)₂ (Compound 18)

Compound 17 (0.094 mmol) and LiOH (2.28 mmol) are stirred in water (20 mL) at room temperature for 6 h, followed by acidification with 1N HCl to pH 3. The crude product is extracted into DCM and crystallized from chilled DCM-ether to give the product.

### Example 16

### Preparation of 40 k 4-Arm PEG-Asp(Leu-Toyocamycin)₂ (Compound 19)

EDC.HCl (380.7 mg, 1.98 mmol) is added to a mixture of 18 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 17

### Preparation of 40 k 4-Arm PEG-IDA(OMe)₂ (Compound 21)

A mixture of 40 k 4-arm PEG acid (15, 1.47 g, 0.037 mmol), compound 20 (0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 18

### Preparation of 40 k 4-Arm PEG-IDA(OH)₂ (Compound 22)

Compound 21 (0.094 mmol) and LiOH (2.28 mmol) are stirred in water (20 mL) at room temperature for 6 h, followed by acidification with 1N HCl to pH 3. The crude product is extracted into DCM and crystallized from chilled DCM-ether to give the product.

### Example 19

### Preparation of 40 k 4-Arm PEG-IDA(Leu-Toyocamycin)₂ (Compound 23)

EDC.HCl (380.7 mg, 1.98 mmol) is added to a mixture of 22 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 20

### Preparation of 40 k 4-Arm PEG-Amide-Leu-Toyocamycin (Compound 25)

EDC.HCl (380.7 mg, 1.98 mmol) is added to a mixture of 24 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 21

### Preparation of 40 k 4-Arm PEG-Amide-Aib-Toyocamycin (Compound 26)

EDC.HCl (380.7 mg, 1.98 mmol) is added to a mixture of 24 (0.025 mmol), 9 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 22

### Preparation of 40 k 4-Star PEG-Carbamate-Leu-Toyocamycin (Compound 28)

A mixture of 40 k 4-star SC-PEG (27, 1.47 g, 0.037 mmol), Leu-Toyocamycin TFA salt (4, 0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 23

### Preparation of 40 k 4-Star PEG-Carbamate-Aib-Toyocamycin (Compound 29)

A mixture of 40 k 4-star SC-PEG (27, 1.47 g, 0.037 mmol), Aib-Toyocamycin TFA salt (9, 0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 24

### Preparation of 40 k 4-Star PEG-Asp(OMe)₂ (Compound 31)

A mixture of 40 k 4-star PEG acid (30, 0.037 mmol), L-aspartic acid dimethylester.HCl (16, 0.26 mmol), EDC.HCl (0.52 mmol), and DMAP (0.52 mmol) in anhydrous DCM (15 mL) is stirred at room temperature overnight. The solvent is removed and the residue crystallized from 2-propanol to give the product.

### Example 25

### Preparation of 40 k 4-Star PEG-Asp(OH)₂ (Compound 32)

Compound 31 (0.094 mmol) and LiOH (2.28 mmol) are stirred in water (20 mL) at room temperature for 6 h, followed by acidification with 1N HCl to pH 3. The crude product is extracted into DCM and crystallized from chilled DCM-ether to give the product.

### Example 26

### Preparation of 40 k 4-Star PEG-Asp(Leu-Toyocamycin)₂ (Compound 33)

EDC.HCl (380.7 mg, 1.98 mmol) is added to a mixture of 32 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 27

### Preparation of 40 k 4-Star PEG-IDA(OMe)₂ (Compound 34)

A mixture of 40 k 4-star PEG acid (30, 1.47 g, 0.037 mmol), compound 20 (0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 28

### Preparation of 40 k 4-Star PEG-IDA(OH)₂ (Compound 35)

Compound 34 (0.094 mmol) and LiOH (2.28 mmol) are stirred in water (20 mL) at room temperature for 6 h, followed by acidification with 1N HCl to pH 3. The crude product is extracted into DCM and crystallized from chilled DCM-ether to give the product.

### Example 29

### Preparation of 40 k 4-Star PEG-IDA(Leu-Toyocamycin)₂ (Compound 36)

EDC.HCl (380.7 mg, 1.98 mmol) is added to a mixture of 35 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 30

### Preparation of 40 k 8-Arm PEG-Carbamate-Leu-Toyocamycin (Compound 38)

A mixture of 40 k 8-arm SC-PEG (37, 0.019 mmol), Leu-Toyocamycin TFA salt (4, 0.26 mmol), DIEA (0.514 mmol), and DMAP (0.0735 mmol) in a mixture of anhydrous

DCM (15 mL) is stirred at 0° C. to room temperature overnight. The reaction mixture is concentrated in vacuo and the residue is recrystallized from DMF/IPA twice to give the product.

### Example 31

### Preparation of 40 k 8-Arm PEG-carbamate-Aib-Toyocamycin (Compound 39)

A mixture of 40 k 8-arm SC-PEG (37, 0.019 mmol), Aib-Toyocamycin TFA salt (9, 0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 32

### Preparation of 40 k 8-Arm PEG-Asp(OMe)₂ (Compound 41)

A mixture of 40 k 8-arm PEG acid (40, 0.019 mmol), L-aspartic acid dimethylester.HCl (16, 0.26 mmol), EDC.HCl (0.52 mmol), and DMAP (0.52 mmol) in anhydrous DCM (15 mL) is stirred at room temperature overnight. The solvent is removed and the residue crystallized from 2-propanol to give the product.

### Example 33

### Preparation of 40 k 8-Arm PEG-Asp(OH)₂ (Compound 42)

Compound 41 (0.094 mmol) and LiOH (2.28 mmol) are stirred in water (20 mL) at room temperature for 6 h, followed by acidification with 1N HCl to pH 3. The crude product is extracted into DCM and crystallized from chilled DCM-ether to give the product.

### Example 34

### Preparation of 40 k 8-Arm PEG-Asp(Leu-Toyocamycin)₂ (Compound 43)

EDC.HCl (380.7 mg, 1.98 mmol) is added to a mixture of 42 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 35

### Preparation of 40 k 8-Arm PEG-IDA(OMe)₂ (Compound 44)

A mixture of 40 k 8-arm PEG acid (40, 1.47 g, 0.037 mmol), compound 20 (0.26 mmol), DIEA (0.514 mmol), and DMAP (0.0735 mmol) in a mixture of anhydrous DCM (15 mL) is stirred at 0° C. to room temperature overnight. The reaction mixture is concentrated in vacuo and the residue is recrystallized from DMF/IPA twice to give the product.

### Example 36

### Preparation of 40 k 8-Arm PEG-IDA(OH)₂ (Compound 45)

Compound 44 (0.094 mmol) and LiOH (2.28 mmol) are stirred in water (20 mL) at room temperature for 6 h, followed by acidification with 1N HCl to pH 3. The crude product is extracted into DCM and crystallized from chilled DCM-ether to give the product.

### Example 37

### Preparation of 40 k 8-Arm PEG-IDA(Leu-Toyocamycin)₂ (Compound 46)

EDC.HCl (380.7 mg, 1.98 mmol) is added to a mixture of 45 (0.025 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 38

### Preparation of 20 k mPEG-Leu-Toyocamycin (Compound 48)

EDC.HCl (380.7 mg, 1.98 mmol) is added to a mixture of 20 k mPEG-SC (47, 0.050 mmol), 4 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 39

### Preparation of 20 k mPEG-Aib-Toyocamycin (Compound 49)

EDC.HCl (380.7 mg, 1.98 mmol) is added to a mixture of 20 k mPEG-SC (47, 0.050 mmol), 9 (1.24 mmol), NMM (3.97 mmol), and HOBT (1.49 mmol) in anhydrous DCM (10 mL) and DMF (10 mL) at 0° C. The mixture is stirred at 0° C. to room temperature overnight. The solvent is removed and the residue recrystallized from IPA to give the product.

### Example 40

### Preparation of Boc-Ext-Urea-Toyocamycin (Compound 52)

Anhydrous DCM (3 mL) was added to a solution of toyocamycin (1, 0.742 mmol) in anhydrous DMF (3 mL) at 0° C., followed by addition of carbodiimidazole (50, 0.742 mmol) and the mixture was stirred for 3 hours, followed by addition of Boc-ext-NH₂ (51, 0.742 mmol). The reaction mixture was allowed to warm to room temperature with stirring overnight. The mixture was concentrated in vacuo and the residue was purified by prep HPLC using C18 column to give the product. ¹H and ¹³C NMR confirmed the structures.

### Example 41

### Preparation of TFA Ext-Urea-Toyocamycin (Compound 53)

A mixture of Boc-ext-urea-Toyocamycin (52, 0.2 mmol) and anhydrous DCM-TFA (1 mL/1 mL) was stirred at 0° C. for 30 minutes and the reaction progress was monitored by TLC. Upon completion of the reaction, the solvent was removed in vacuo and the residue was washed with anhydrous ether several times and dried in vacuo. HPLC confirmed the completion of reaction and the crude product was used for the next step without further purification.

### Example 42

### Preparation of 40 k 4-Arm PEG-Carbamate-Ext-Urea-Toyocamycin (Compound 54)

A mixture of 40 k 4-arm SC-PEG (5, 800 mg, 0.02 mmol), ext-urea-Toyocamycin TFA salt (53, 0.2 mmol), DIEA (0.2 mmol), and DMAP (0.04 mmol) in a mixture of anhydrous DCM-DMF (6 mL/1 mL) was stirred at 0° C. to room temperature overnight. The reaction mixture was concentrated in vacuo and the residue was recrystallized from DMF/IPA twice to give 700 mg of product. ¹³C NMR confirmed that the structure of the product. The content of toyocamycin measured by UV was about 2.3-2.9% wt/wt and the purity measure by HPLC was 100%.

### Example 43

### Regeneration of Parent Molecules from Compounds of Formula (I)

The rate of hydrolysis was measured by monitoring disappearance of polymer conjugates and appearance of the parent molecule by HPLC using the procedure for example as described in Example 4 in PBS and in rat plasma. The stability of compounds 6, 10, and 54 are set forth in the table below. The result indicates that the polymeric conjugates of the present invention are quite stable in PBS solution but release the parent drug in vivo.

| | | |
|---|---|---|
| Compound No. | t_{1/2} in at plasma | t_{1/2} in PBS |

| | | | |
|---|---|---|---|
| 6 | 2 | minutes | 12.8 hour |
| 10 | 4.1 | hour | stable |

| | | |
|---|---|---|
| 54 | stable | stable |

### Example 44

### Efficacy on Tumor Growth Inhibition in Mice Xenografted with Human Melanoma

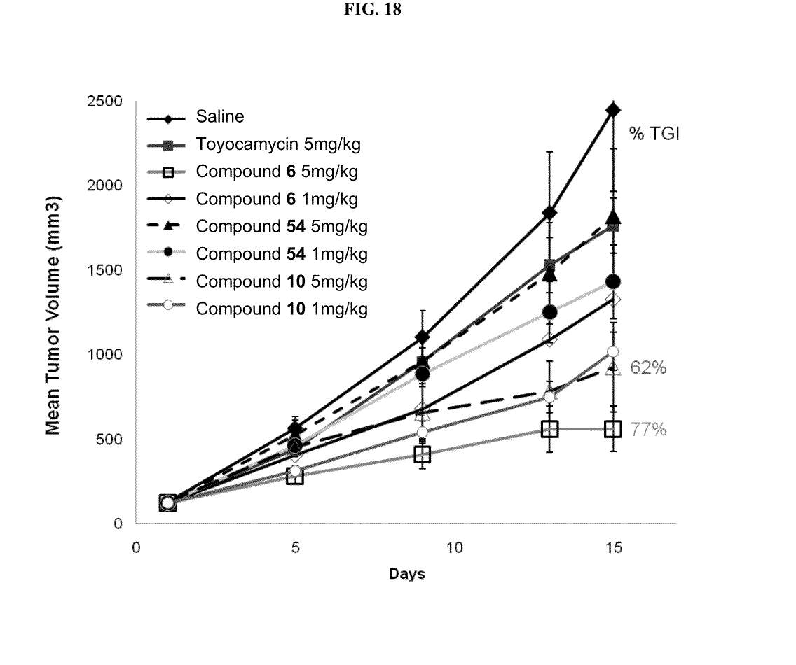

The antitumor efficacies of compounds 6, 10, and 54 evaluated in human melanoma xenografted mice. Xenograft tumors were established in mice by injecting human melanoma cells (A375). The mice were treated with toyocamycin i.v. at 5 mg/kg/dose, or compounds 6, 10, and 54 at 1 or 5 mg/kg/dose (based on the amount of toyocamycin) at day 1, 5, 9, and 13. Control group mice received saline solution. Both compounds 6 and 10 inhibited tumor growth significantly compared to toyocamycin. The results are shown in FIG. 18. Tail vein necrosis was observed in only one mouse from among those treated with compounds 6 and 10. Both compounds 6 and 10 were more effective in the treatment of melanoma than untreated, toyocamycin, or 54 which did not have provide any release of toyocamycin in rat plasma or PBS, as provided in Example 43. No animal showed tail vein necrosis in the second set of study.

1. A compound containing an adenosine nucleoside having Formula (I): 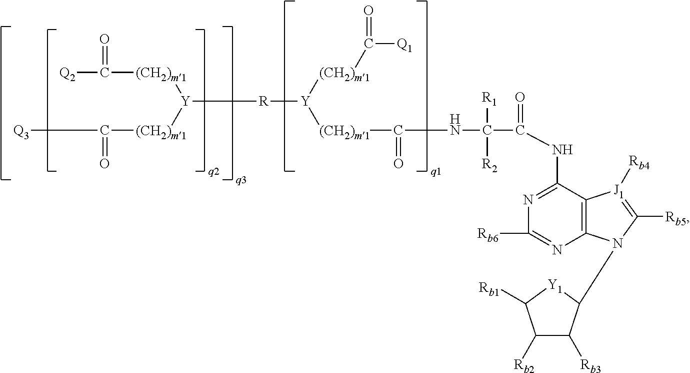
wherein
R is a substantially non-antigenic polymer having from about one to about 32 polymer arms;
Y is —NHCH— or N;
Q₁, Q₂, and Q₃, in each occurrence, are independently OH, a leaving group, 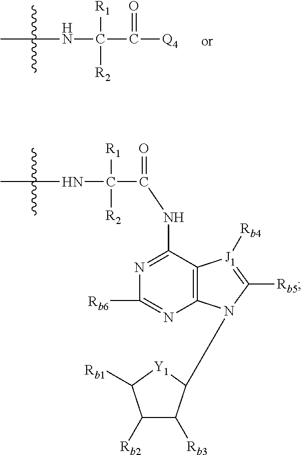
Q₄, in each occurrence, is independently OH or a leaving group;
R₁, in each occurrence, is independently H, C₁₋₁₀ alkyl, C₃₋₁₀ branched alkyl, C₃₋₈ cycloalkyl, C₂₋₁₀ alkenyl or C₂₋₁₀ alkynyl;
R₂, in each occurrence, is independently C₁₋₁₀ alkyl, C₃₋₁₀ branched alkyl, C₃₋₈ cycloalkyl, C₂₋₁₀ alkenyl or C₂₋₁₀ alkynyl;
J₁, in each occurrence, is independently C or N;
Y₁, in each occurrence, is independently O, S, or CH₂;
R_{b1}, in each occurrence, is independently hydrogen, hydroxyl, C₂₋₁₀ alkenyl, C₂₋₁₀ alkynyl, —(CH₂)ₘ₂—OR_{c1} or —(CH₂)ₘ₂—R′_{c1};
R_{b2}, in each occurrence, is independently hydrogen, hydroxyl, C₂₋₁₀ alkenyl, C₂₋₁₀ alkynyl, C₂₋₁₀ alkenyloxy, C₃₋₁₀ alkyloxy, halogen, azido, amino, or OR_{c2};
R_{b3}, in each occurrence, is independently hydrogen, hydroxyl, C₂₋₁₀ alkenyl, C₂₋₁₀ alkynyl, C₂₋₁₀ alkenyloxy, C₃₋₁₀ alkyloxy, halogen (F), azido, amino, or OR_{c3};
R_{b4}, when J₁ is carbon, in each occurrence, is independently hydrogen, halogen, C₁₋₁₀ alkyl, aryl, aralkyl, C₂₋₁₀ alkenyl, C₂₋₁₀ alkynyl, C₁₋₁₀ alkoxy, cyano, cyanoalkyl, —C(═O)NH₂, carboxyamido, aryloxy, amino, alkylamino, arylamino, aralkylamino, alkylthio, or arylthio; and when J₁ is nitrogen, R_{b4} is null;
R_{b5}, in each occurrence, is independently hydrogen, amine, halogen, C₁₋₁₀ alkyl, alkylamino, alkylthio, —NH—NH₂, or azido;
R_{b6}, in each occurrence, is independently hydrogen, C₁₋₁₀ alkyl (lower alkyl), halogen (F, Cl), C₁₋₁₀ alkoxy, or C₁₋₁₀ alkylthio;
R_{c1}, in each occurrence, is independently hydrogen, C₁₋₁₀ acyl, monophosphate, diphosphate, triphosphate, C₁₋₁₀ alkyl, C₃₋₈ cycloalkyl, C₂₋₁₀ alkenyl, C₂₋₁₀ alkynyl, or a substantially non-antigenci polymer;
R′_{c1}, in each occurrence, is independently hydrogen, hydroxyl, lower alkyl esters or carbonate esters thereof, C₁₋₁₀ alkyl, C₁₋₁₀ alkoxy, amino, azido, halogen or a substantially non-antigenci polymer;
R_{c2}, in each occurrence, is independently hydrogen, C₁₋₁₀ acyl, monophosphate, diphosphate, triphosphate C₁₋₁₀ alkyl, C₃₋₈ cycloalkyl, C₂₋₁₀ alkenyl, C₂₋₁₀ alkynyl, or a substantially non-antigenic polymer;
R_{c3}, in each occurrence, is independently hydrogen, C₁₋₁₀ acyl, monophosphate, diphosphate, triphosphate, —CH₂CH₂OH, or CH₂CH₂F, C₁₋₁₀ alkyl, C₃₋₈ cycloalkyl, C₂₋₁₀ alkenyl, C₂₋₁₀ alkynyl, or a substantially non-antigenic polymer;
(m1) and (m′1) are independently zero, 1, or 2, provided that, when Y is N, (m1) and (m′1) are independently 1 or 2;
(m2) is an integer from about 1 to about 4;
(q1) and (q2) are independently zero or 1; and
(q3) is zero or a positive integer of from about 1 to about 31.

2. A compound of claim 1, wherein the number of the adenine nucleosides contained in the compound of Formula (I) ranges from about 1 to about 64.

3. A compound of claim 1, wherein R has a total number average molecular weight of from about 2,000 to about 100,000 daltons.

4. A compound of claim 1, wherein R comprises a polyalkylene oxide.

5. A compound of claim 1 selected from the group consisting of: 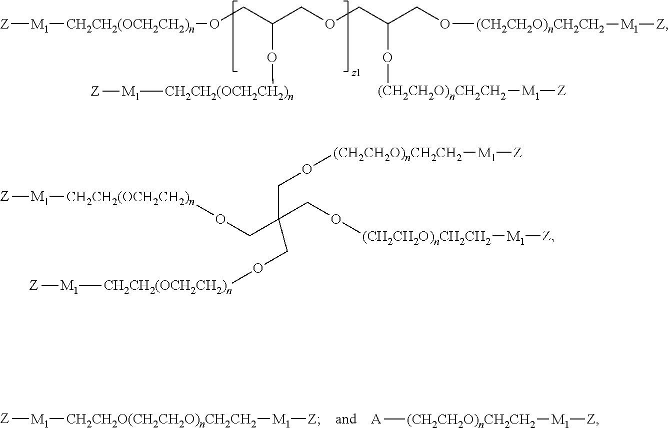
wherein
M₁ is independently O, or S;
Z, each occurrence, is independently H, 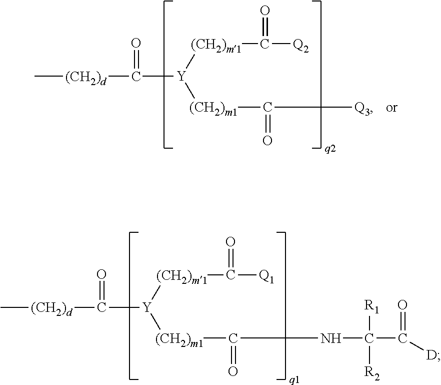
Y, in each occurrence, is —NHCH— or N;
Q₁, Q₂, and Q₃, in each occurrence, are independently OH, a leaving group, 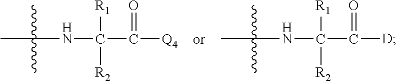
Q₄, in each occurrence, is independently OH or a leaving group;
D is 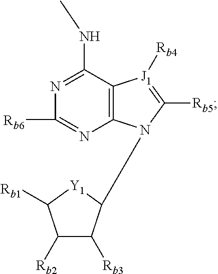
A is OH, C₁₋₆ alkoxy, COOH, or NH₂;
(d) is zero or a positive integer from about 1 to about 10;
(z1) is zero or a positive integer from 1 to about 29; and
(n) is a positive integer from about 10 to about 2,300 so that the total number average molecular weight of R ranges from about 2,000 to about 100,000 daltons,
provided that at least one of Z is

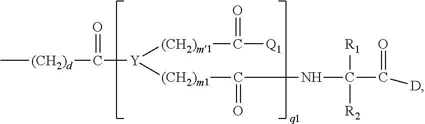

6. A compound of claim 5, wherein
Z is H, 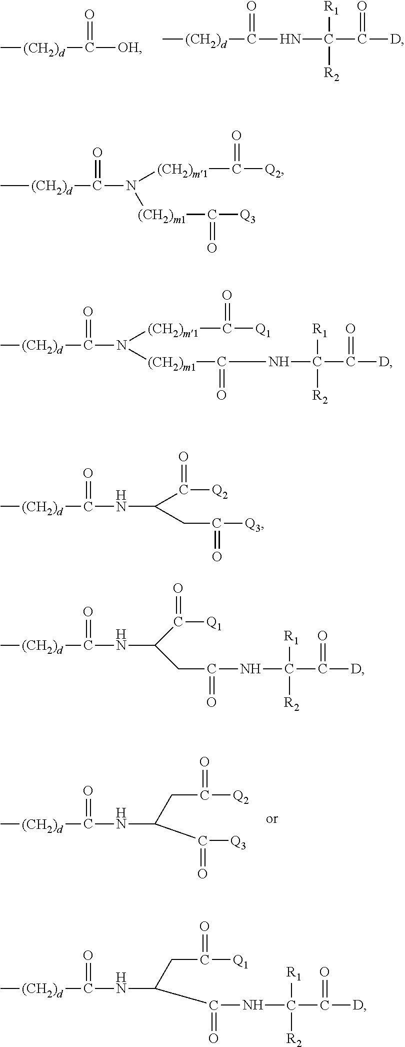
wherein
(d) is zero, 1 or 2;
(m1) and (m′1) are 1 or 2; and
at least one of Z is:

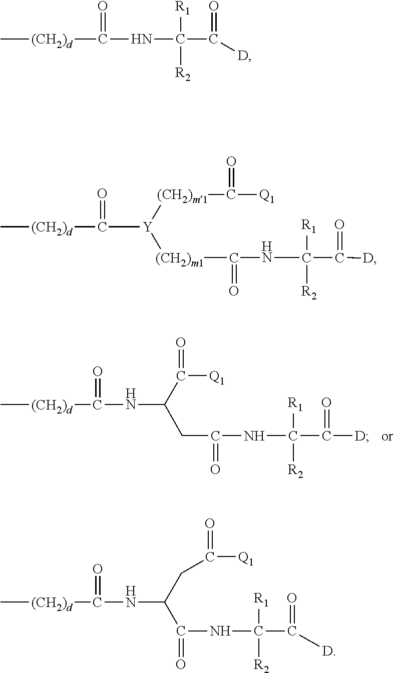

7. A compound of claim 5, wherein (q1) and (q2) are both zero; and Z, in each occurrence, is independently H, 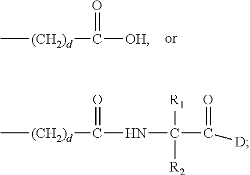
(d) is zero, 1 or 2; and
at least one of Z is

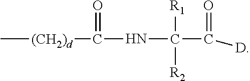

8. (canceled)

9. A compound of claim 1, wherein
Y₁ is O;
J₁ is carbon;
R_{b1}, in each occurrence, is independently hydrogen, hydroxyl, or —CH₂—OR_{c1};
R_{b2}, in each occurrence, is independently hydrogen or OR_{c2};
R_{b3}, in each occurrence, is independently hydrogen, or OR_{c3};
R_{b4}, in each occurrence, is independently hydrogen, cyano or —C(═O)NH₂;
R_{b5} is hydrogen, amine, or —NH—NH₂;
R_{b6} is hydrogen, F, or Cl; and
R_{c1}, R_{c2}, and R_{c3}, in each occurrence, are independently hydrogen, acyl, monophosphate, diphosphate, or triphosphate.

10-11. (canceled)

12. A compound of claim 5, wherein D comprises: 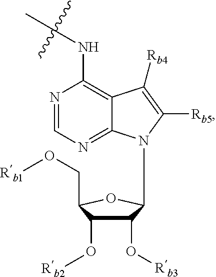
wherein
R′_{b1}, R′_{b2} and R′_{b3} are independently hydrogen, monophosphate, diphosphate, or triphosphate;
R_{b4} is —CN, —C(═O)NH₂, or hydrogen; and
R_{b5} is hydrogen, amine, or —NH—NH₂,
or a pharmaceutical salt thereof.

13. A compound of claim 1, wherein R₁, in each occurrence, is independently H, C₁₋₆ alkyl, C₃₋₆ branched alkyl, or C₃₋₆ cycloalkyl; and R₂, in each occurrence, is independently C₁₋₆ alkyl, C₃₋₆ branched alky or C₃₋₆ cycloalkyl.

14. (canceled)

15. A compound of claim 5, wherein (n) is an integer from about 28 to about 341, so that the total average molecular weight of R ranges from about 5,000 to about 60,000 daltons.

16. A compound of claim 5, wherein (n) is an integer of from about 114 to about 239, so that the total average molecular weight of R ranges from about 20,000 to about 42,000 daltons.

17. A compound of claim 5 selected from the group consisting of: 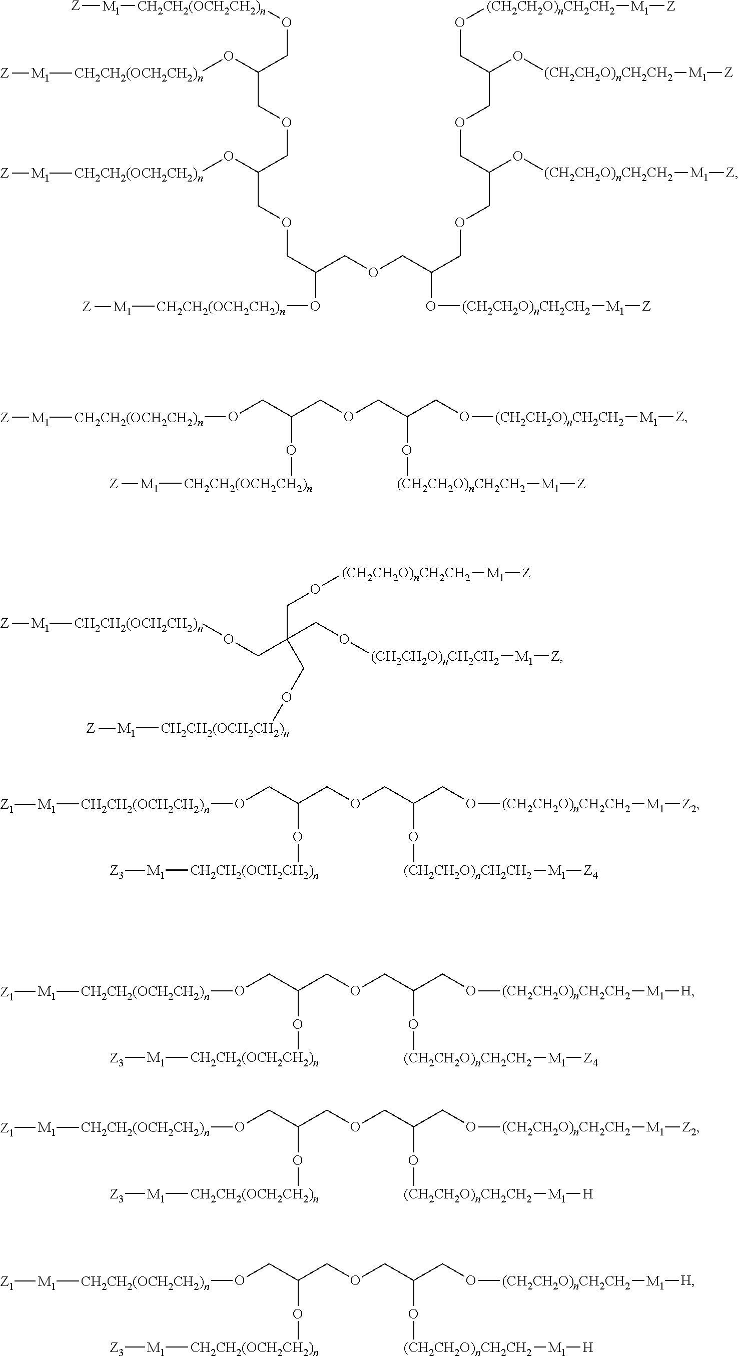 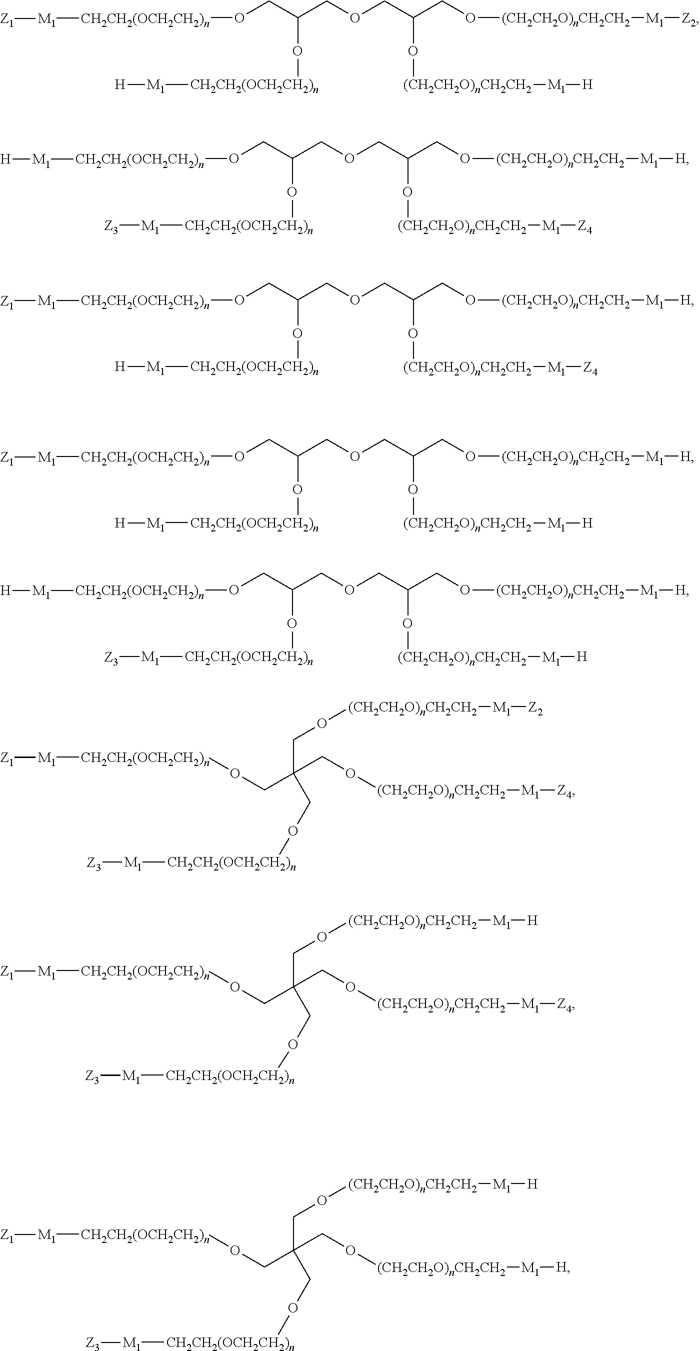 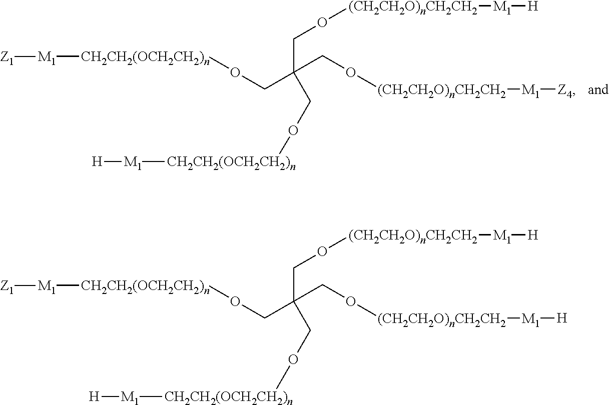
wherein
Z₁, Z₂, Z₃, and Z₄ are selected from the group consisting of: 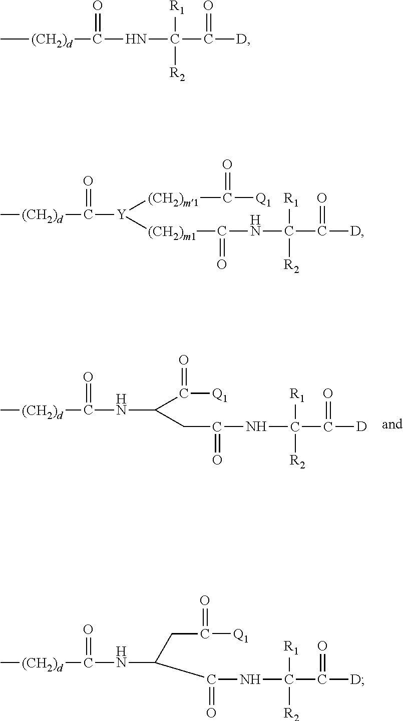
wherein 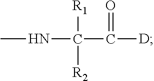
Q₁ is hydroxyl or
(d) is zero, 1 or 2;
(n) is a positive integer of from about 10 to about 2,300 so that the polymeric portion of the compound has the total number average molecular weight of from about 2,000 to about 100,000 daltons; and
all other variables are as previously defined.

18. A compound of claim 17, comprising:

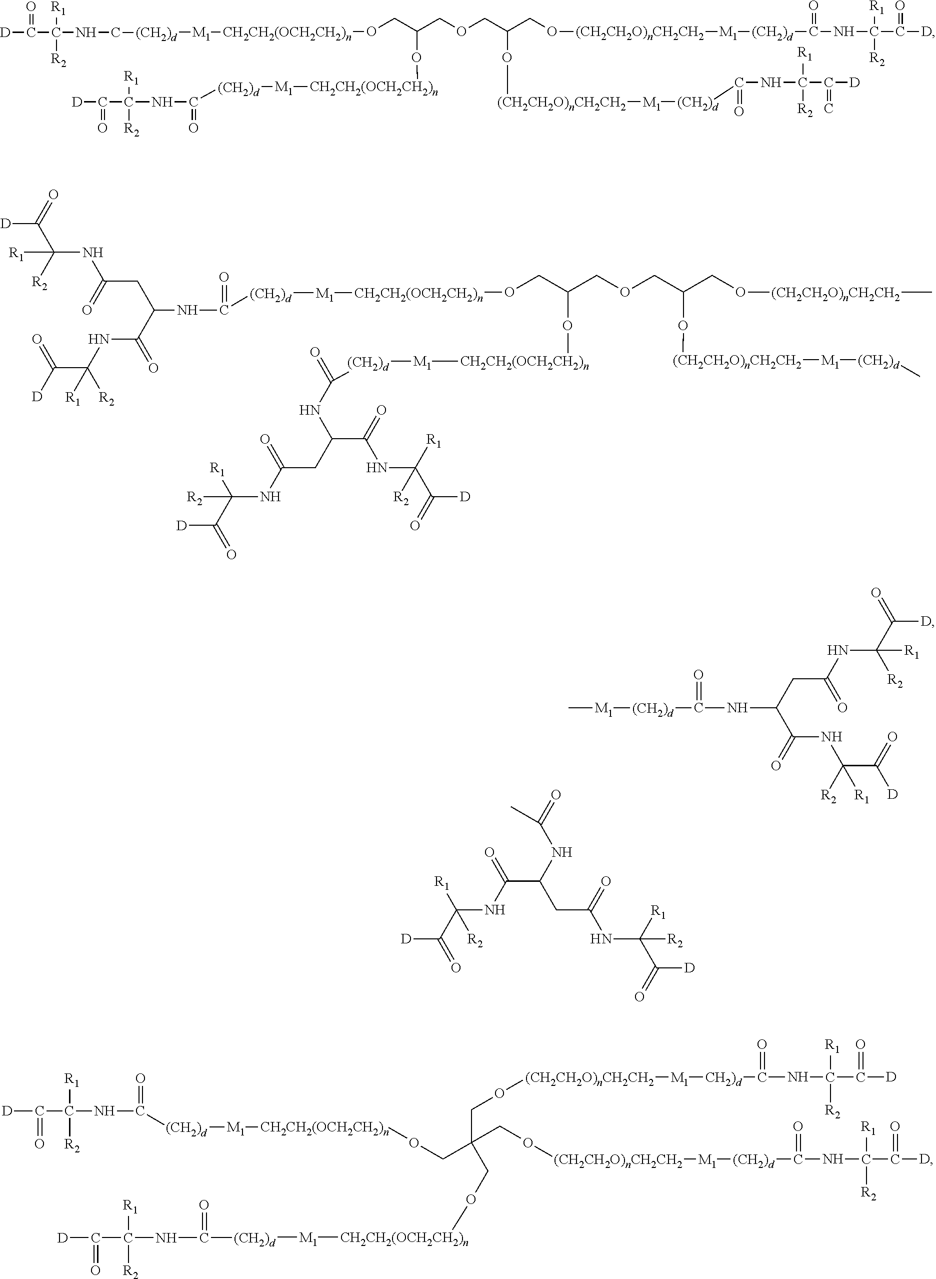

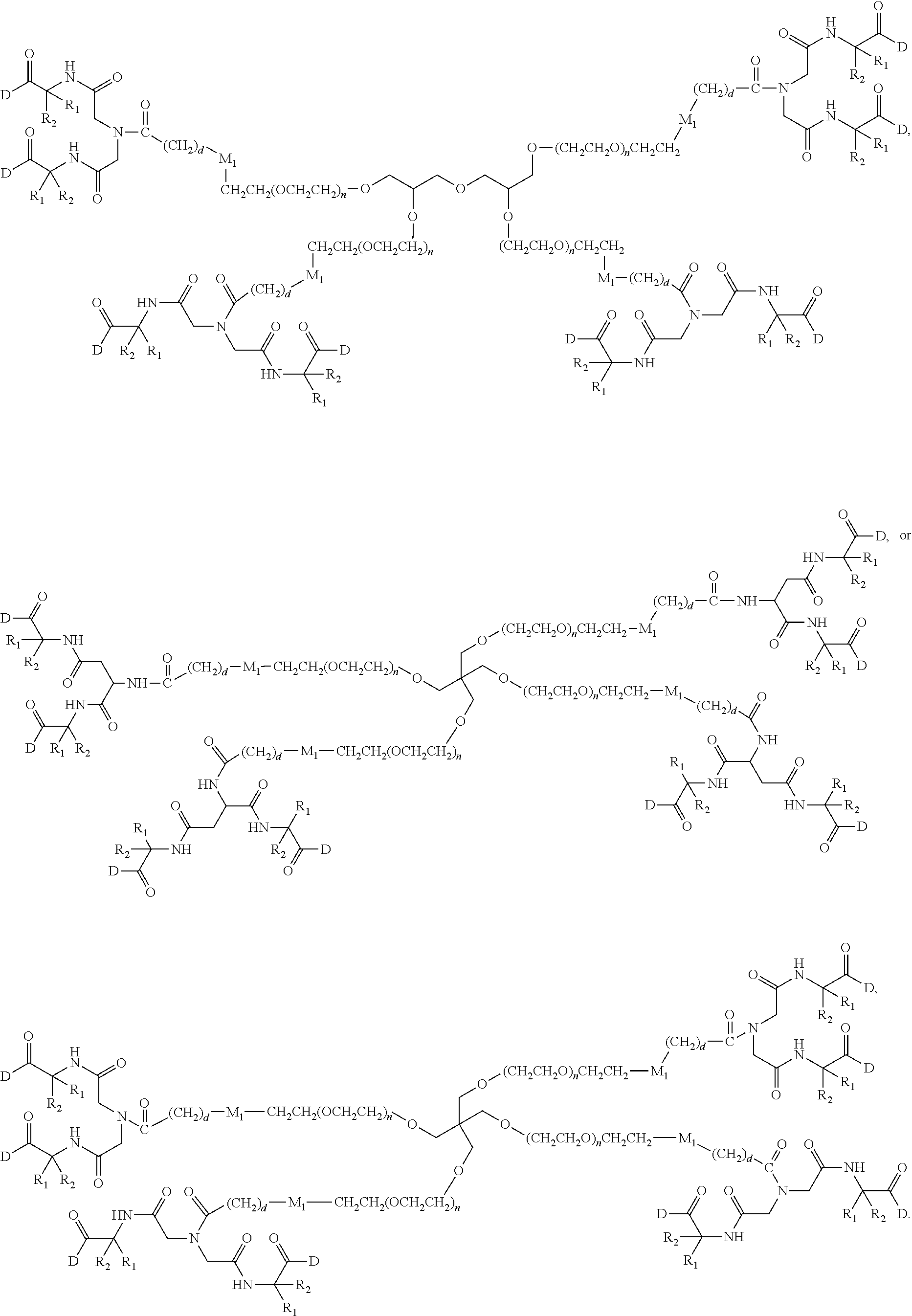

19. A compound of claim 17, selected from the group consisting of:

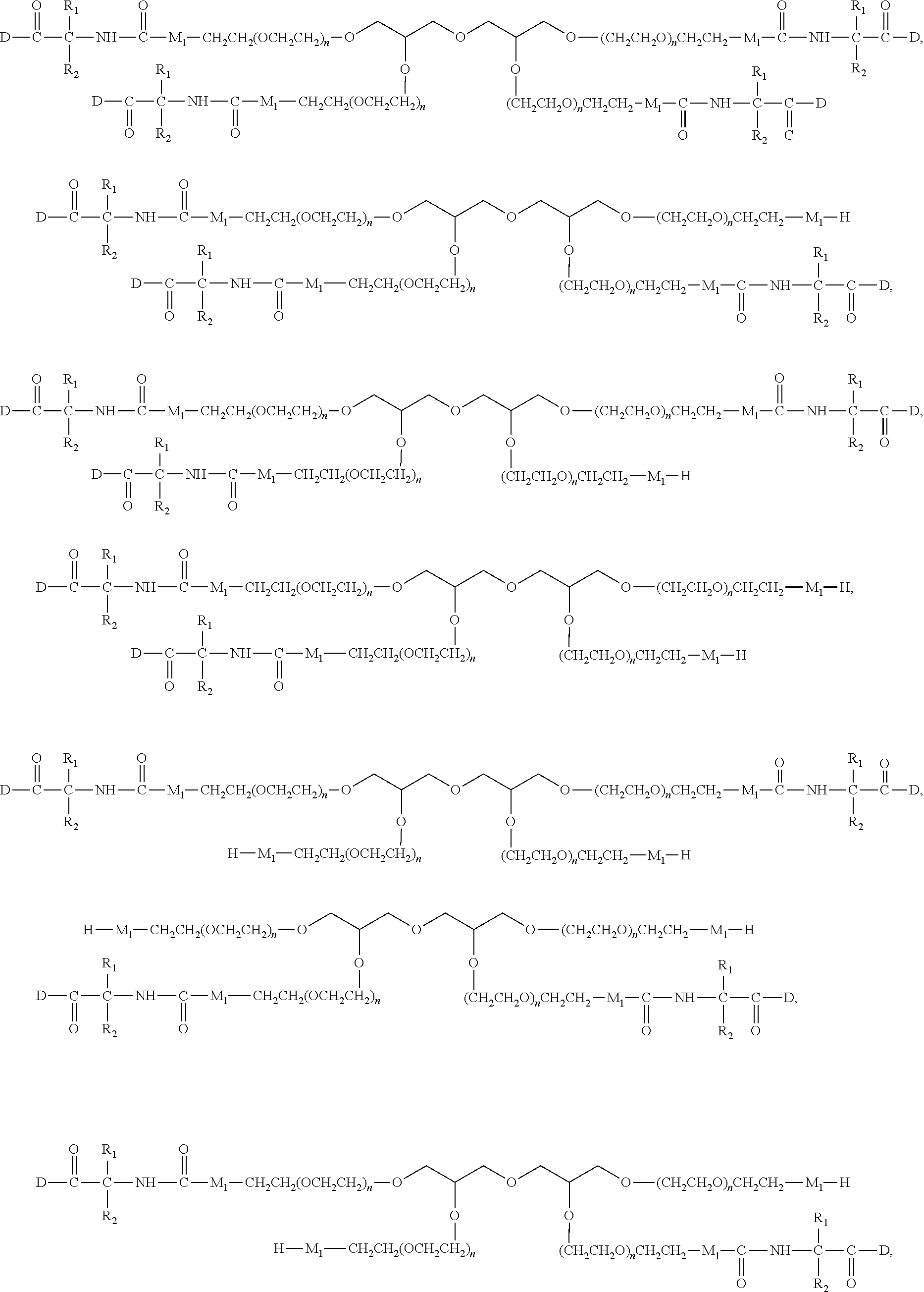

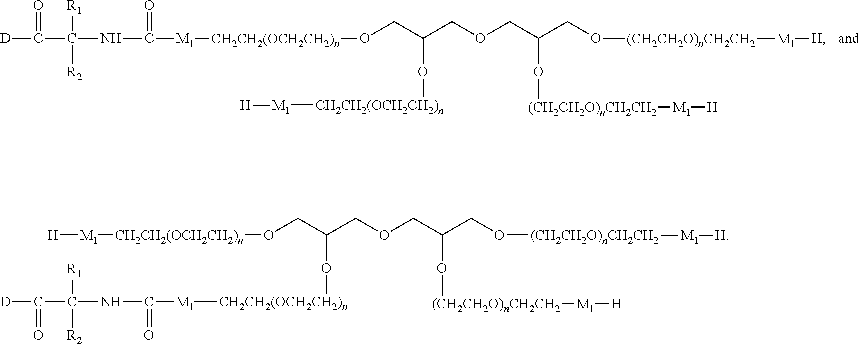

20. A compound of claim 5, wherein, 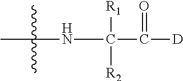
is selected from the group consisting of:

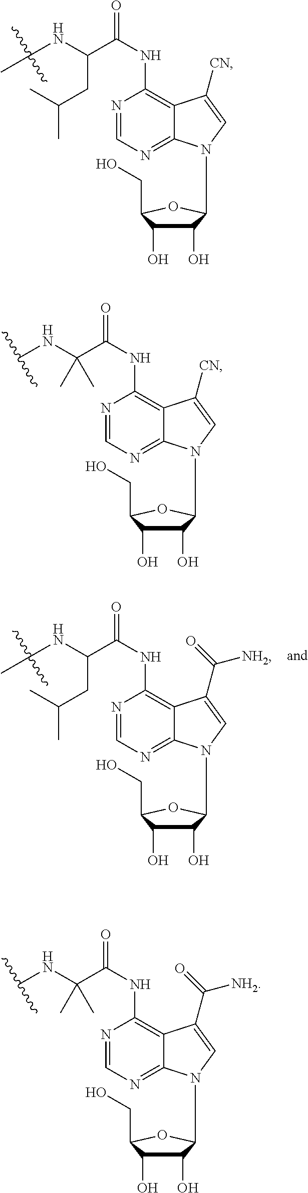

21. A compound of claim 1, selected from the group consisting of: 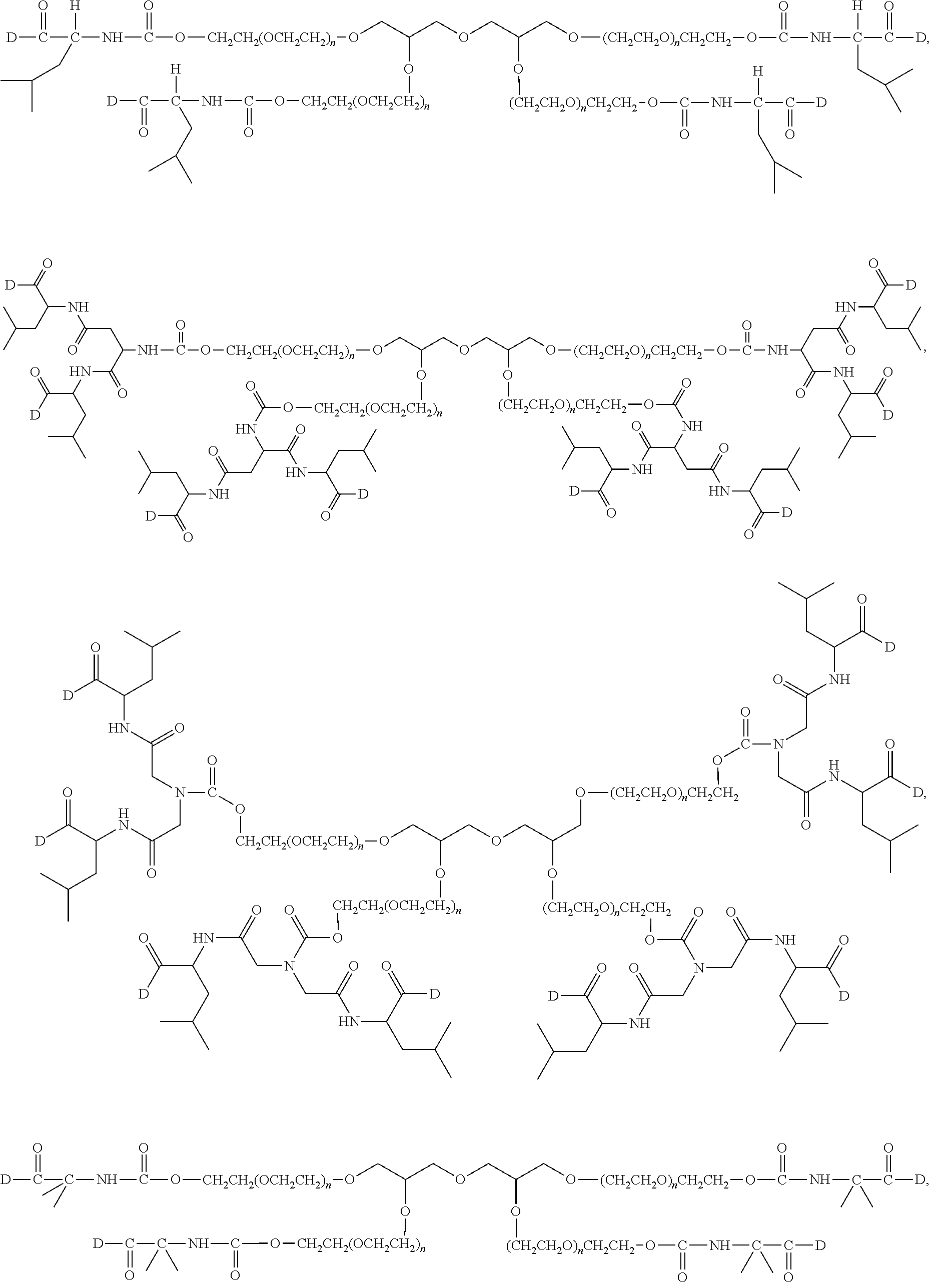 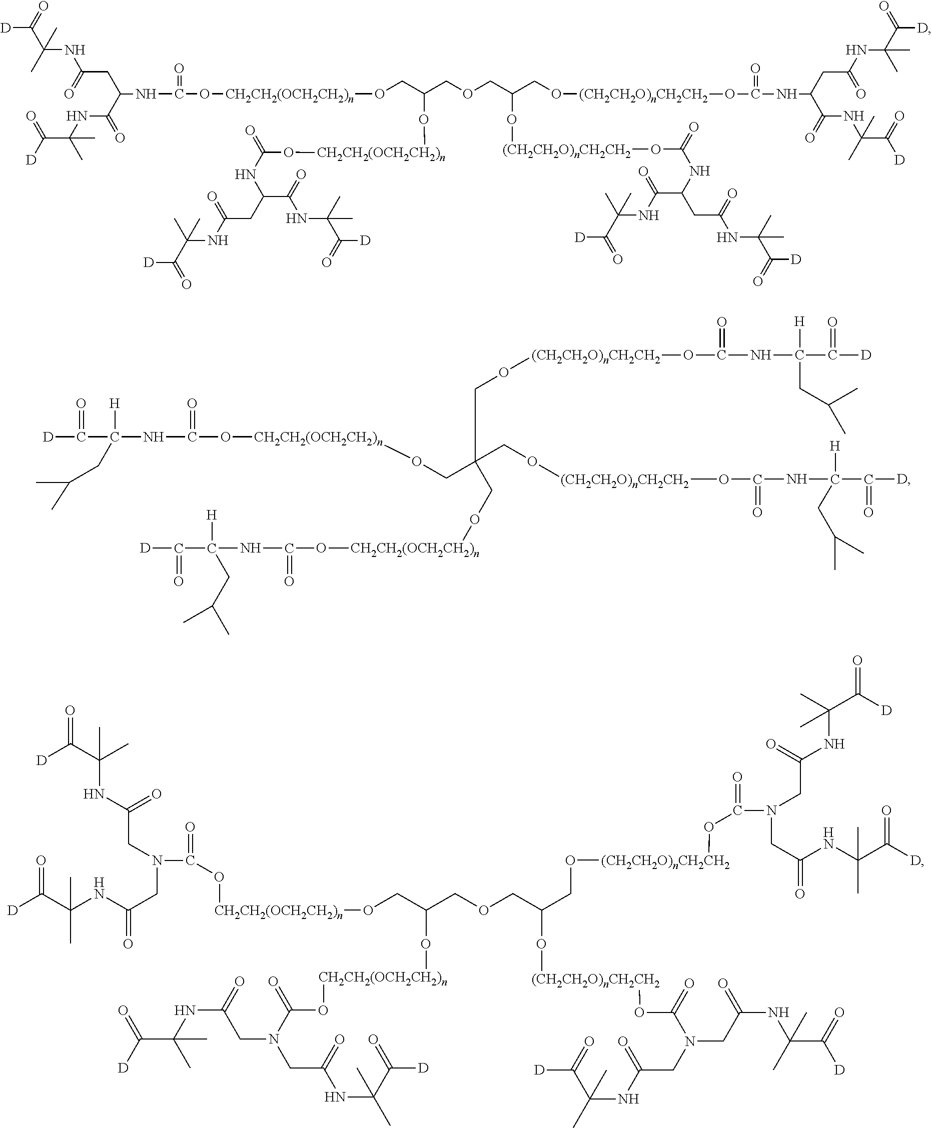 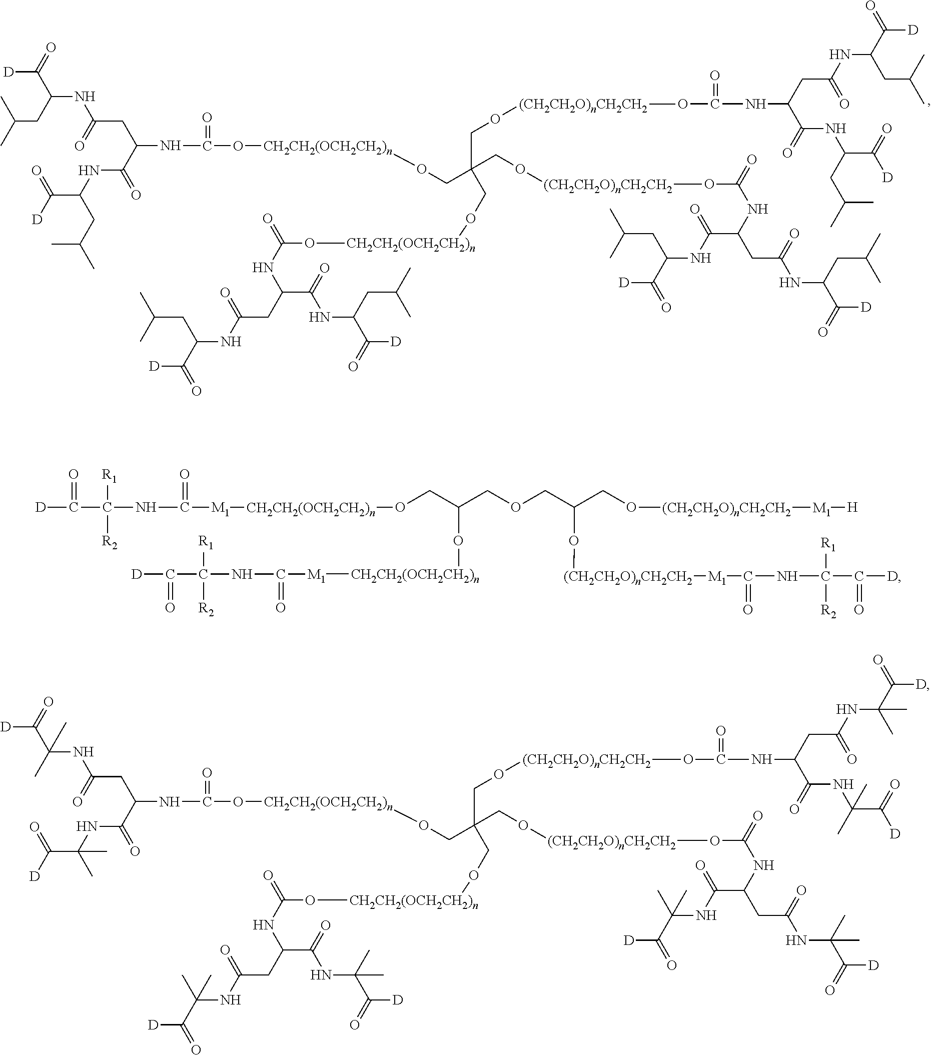 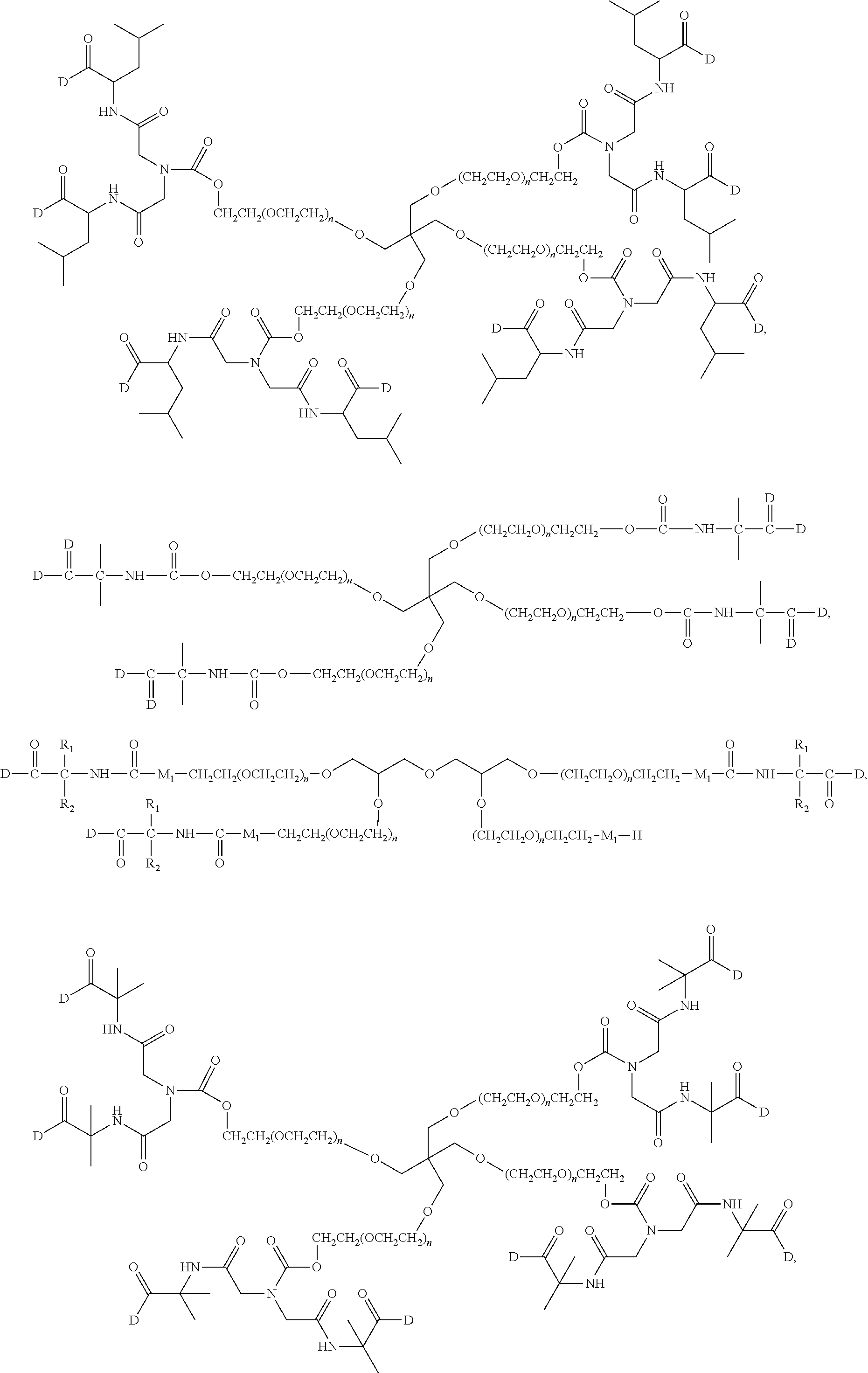 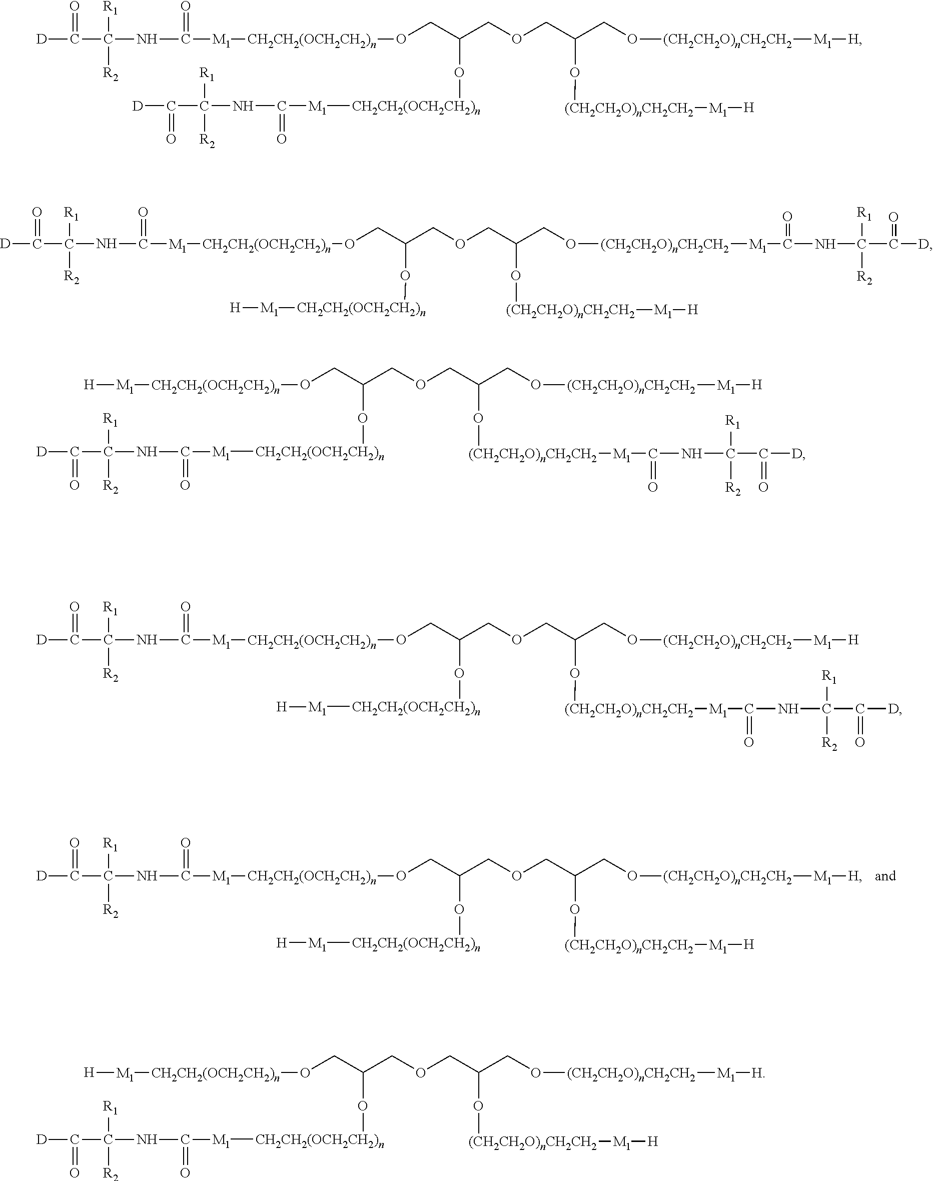
wherein D is 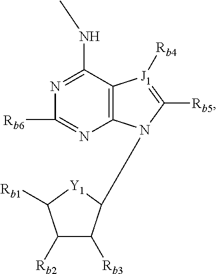
and
all other variables are as previously defined.

22. A method of preparing a compound of Formula (I), comprising:
(a) reacting one equivalent of an adenine nucleoside with one or more equivalents of a bifunctional spacer containing an available carboxylic acid group or activated carboxylic acid group under conditions effective to form an adenine nucleoside-spacer amide intermediate having an available amine group; and
(b) reacting one or more equivalents of the resulting intermediate from step (a) per each polymer arm terminal with one equivalent of an activated polymer under conditions effective to form a compound of Formula (I):

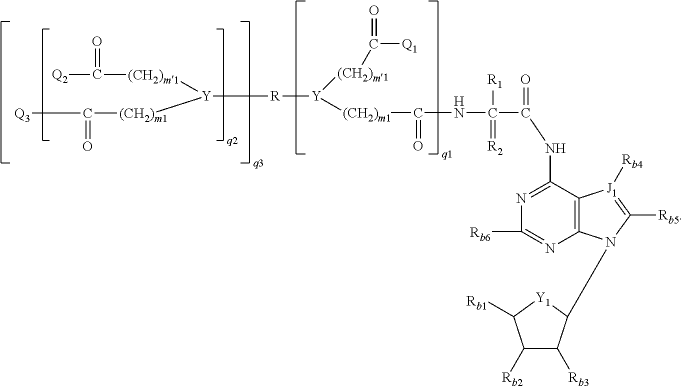

23. A method for treating a cancer, inhibiting the growth or proliferation of cancer cells, treating a viral infection, treating a disease or condition associated with abnormal expression of VEGF in a mammal, comprising administering an effective amount of a compound of claim 1 or a pharmaceutical salt thereof to a mammal in need thereof.

24. The method of claim 23, wherein the administering step comprises administration via the blood stream of the mammal.

25-26. (canceled)
